SEQUENCE LISTING (34 sequences)

SEQ ID NO: 1 - DNA, 104 bp, Artificial Sequence
  N20 is the generalized target sequence of a sgRNA guide RNA
nnnnnnnnnnnnnnnnnnnngttttagagctagaaatagcaagttaaaataaggctagtccgttatcaacttgaaaaagtggcaccgagtcggtgctttttttt

SEQ ID NO: 2 - DNA, 109 bp, Artificial Sequence
  N19 is the target sequence for a guide sgRNA for S. Thermophiles
nnnnnnnnnnnnnnnnnnnguuuuagagcuguaggauccaacagcgaguuaaaauaaggcuuaguccguacucaacuugaaaagguggcaccgauucgguguuuuuuuu

SEQ ID NO: 3 - DNA, 145 bp, Artificial Sequence
  N24 is the generalized target for the guide sgRNA of N. Meningitides
nnnnnnnnnnnnnnnnnnnnnnnnguuguagcucccuuucucauuucggaaacgaaaugagaaccguugcuacaauaaggccgucugaaaagaugugccgcaacgcucugccccuuaaagcuucugcuuuaaggggcaucguuua

SEQ ID NO: 4 - DNA, 656 bp, Artificial Sequence
  Generalized ATU6-26 PROMOTER, S. PYOGENES SGRNA, AND TERMINATOR REGION
gcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnngttttagagctagaaatagcaagttaaaataaggctagtccgttatcaacttgaaaaagtggcaccgagtcggtgcttttttttgcaaaattttccagatcgatttcttcttcctctgttcttcggcgttcaatttctgggtttttctcttcgttttctgtaactgaaacctaaaatttgacctaaaaaa

SEQ ID NO: 5 - DNA, 661 bp, Artificial Sequence
  ATU6-26 PROMOTER, S. THERMOPHILES SGRNA, AND TERMINATOR REGION
gcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnngttttagagctgtaggatccaacagcgagttaaaataaggcttagtccgtactcaacttgaaaaggtggcaccgattcggtgttttttttgcaaaattttccagatcgatttcttcttcctctgttcttcggcgttcaatttctgggtttttctcttcgttttctgtaactgaaacctaaaatttgacctaaaaaa

SEQ ID NO: 6 - DNA, 705 bp, Artificial Sequence
  ATU6-26 PROMOTER, NEISSERIA MENINGITIDES SGRNA, AND TERMINATOR REGION
gcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgnnnnnnnnnnnnnnnnnnnnnnngttgtagctccctttctcatttcggaaacgaaatgagaaccgttgctacaataaggccgtctgaaaagatgtgccgcaacgctctgccccttaaagcttctgctttaaggggcatcgtttattttttttgcaaaattttccagatcgatttcttcttcctctgttcttcggcgttcaatttctgggtttttctcttcgttttctgtaactgaaacctaaaatttgacctaaaaaa

SEQ ID NO: 7 - DNA, 8433 bp, Artificial Sequence
  PCAMBIA1300 binary plasmid with BAR selectable marker
gaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcatcagatttcggtgacgggcaggaccggacggggcggtactggcaggctgaagtccagctgccagaaacccacgtcatgccagttcccgtgcttgaagccggccgcccgcagcatgccgcgtggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgattcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 8 - protein, 185 aa, Artificial Sequence
  Synthetic Construct
SSDFGDGQDRTGRYWQAEVQLPETHVMPVPVLEAGRPQHAAWGISERLVHAHARVVGQPDDSDHALEALCLQGLQQVGVERGAQSRPLVAGGDVHGRFGRPVVGVACLPGARVGDAGDLAVHLGDEPGIALPQTDEVVRPLLRFLRLGTEVDRACLDVVVDDGADRRHVRLGGTADVGRASFWAH

SEQ ID NO: 9 - DNA, 976 bp, Artificial Sequence
  CaMV 35S Promoter and caster bean catalase gene cassette for expressing coding regions
gaattcatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctctcgaccaggtaaatttctagtttttctccttcattttcttggttaggacccttttctctttttatttttttgagctttgatctttctttaaactgatctattttttaattgattggttatggcgcaaatattacatagctttaactgataatctgattactttatttcgtgtgtctatgatgatgatgatgttacaggtctcgagaaatttgagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactggatccaaatttggtaccataaagcttaaaa

SEQ ID NO: 10 - DNA, 1548 bp, Artificial Sequence
  XVE coding seqeunce for regulated gene expression
atatgtcgacgaagctagtcaccatggctatgaaggccctgaccgccaggcagcaggaggtgttcgacctgatcagggaccacatctcccagaccggcatgccaccgaccagggccgagatcgcccagaggctgggcttcaggtccccgaacgctgccgaggagcacctgaaggccctggccaggaagggcgtgatcgagatcgtgtccggagcctccaggggcatcaggctgctgcaagaggaggaggagggcctgccgctggtgggcagggtggctgctggcgagccgtcctccgctccgccgaccgacgtgtccctgggcgacgagctgcacctggacggcgaggacgtggccatggcccacgccgacgccctggacgacttcgacctggacatgctgggcgacggcgactccccaggccctggcttcaccccgcacgactccgctccgtacggtgccctggacatggccgacttcgagttcgagcagatgttcaccgacgccctgggcatcgacgaggctagcatgaggccggagtgcgtggtgccggagacccagtgcgccatgaagaggaaggagaagaaggcccagaaggagaaggacaagctgccggtgtccaccaccaccgtggacgaccacatgccaccgatcatgcaatgcgagccgccacctccggaggctgcccgcatccacgaggtggtgccgaggttcctgtccgacaagctgctggagaccaacaggcagaagaacatcccgcagctgaccgccaaccagcagttcctgatcgccaggctgatctggtatcaggacggctacgagcagccgtccgacgaggacctgaagaggatcacccagacctggcagcaggccgacgacgagaacgaggagtccgacaccccgttcaggcagatcaccgagatgaccatcctgaccgtgcaactgatcgtggagttcgccaagggcctgccaggcttcgccaagatctcccagccggaccagatcaccctgctgaaggcctgctcctccgaggtgatgatgctgagggtggccaggaggtacgacgctgcctccgactccgtgctgttcgccaacaaccaggcctacaccagggacaactaccggaaggctggcatggcctacgtgatcgaggacctgctgcacttctgccggtgcatgtactccatggccctggacaacatccactacgccctgctgaccgccgtggtgatcttctccgacaggccaggcctggagcagccgcagctggtggaggagatccagaggtactacctgaacaccctgaggatctacatcctgaaccagctgtccggctccgccaggtcctccgtgatctacggcaagatcctgtccatcctgtccgagctgaggaccctgggcatgcaaaactccaacatgtgcatctccctgaagctgaagaacaggaagctgccaccgttcctggaggagatctgggacgtggccgacatgtcccacacccagccaccgccgatcctggagtccccgaccaacctgtgagagctcaaaa

SEQ ID NO: 11 - protein, 504 aa, Artificial Sequence
  Synthetic Construct
MAMKALTARQQEVFDLIRDHISQTGMPPTRAEIAQRLGFRSPNAAEEHLKALARKGVIEIVSGASRGIRLLQEEEEGLPLVGRVAAGEPSSAPPTDVSLGDELHLDGEDVAMAHADALDDFDLDMLGDGDSPGPGFTPHDSAPYGALDMADFEFEQMFTDALGIDEASMRPECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 12 - DNA, 743 bp, Artificial Sequence
  LexA regulated gene cassette for XVE regulated expression
aattggatccagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgctcgactctaggatcttcgcaagacccttcctctatataaggaagttcatttcatttggagaggacacgctgaagctagtcctcgagatatattctagaaaatttcccgggctgctttaatgagatatgcgagacgcctatgatcgcatgatatttgctttcaattctgttgtgcacgttgtaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttcctgcaggacacacggtaccacaaagcttatat

SEQ ID NO: 13 - DNA, 4202 bp, Artificial Sequence
  2XNLS-DCAS9 for nuclear localized CRISPR/CAS9 DNA binding protein lacking DNA cleavage catalytic activity
atatctcgagaccaccatgccaaagaagaagcggaaggtagaccctaagaagaagcgcaaggtcgacggcagcgggagcatggacaagaagtacagcatcggcctggccatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctccaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctgtacctgtactacctgcagaacggccgcgacatgtacgtggaccaggagctggacatcaaccgcctgtccgactacgacgtggacgccatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgacggcggaagctctagaaatt

SEQ ID NO: 14 - protein, 1394 aa, Artificial Sequence
  Synthetic Construct
MPKKKRKVDPKKKRKVDGSGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSSR

SEQ ID NO: 15 - DNA, 1796 bp, Artificial Sequence
  Synthetic coding region for soybean DRM2 full length protein
atattctagaatgggaggagatgattctggtttggagagtgacaattttgattggaacactgaagatgagcttgaaattcagaactataactcgtcgtcttcatgtttaacccttcctaatggagatgctgttactggctctggagaggcaagctcgtctgcagttttggctaattccaaggtgcttgatcacttcgtcagcatgggatttagcagagaaatggtttctaaagtaattcaggaatatggtgaggaaaatgaagataaactacttgaagaacttctcacatacaaggccctagaaagttcttcccgtccacagcagcgaattgagccagatccttgttcttcagagaatgcagggagttcttgggatgatttctcagatactgatattttttctgatgatgaagaaattgcaaaaactatgtctgagaatgatgataccttacggtctttggtgaaaatggggtacaagcaggtggaggctttaattgccatagaaagattaggcccaaacgcctcacttgaagaattggtagattttataggtgttgctcaaatggcaaaggctgaagatgctcttctgcctcctcaagaaaagttacaatacaatgactatgctaagtccaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctggacttggtaaaaaatatgtcggccaaatatcgatgatttcccgggatat

SEQ ID NO: 16 - protein, 590 aa, Artificial Sequence
  Synthetic Construct
MGGDDSGLESDNFDWNTEDELEIQNYNSSSSCLTLPNGDAVTGSGEASSSAVLANSKVLDHFVSMGFSREMVSKVIQEYGEENEDKLLEELLTYKALESSSRPQQRIEPDPCSSENAGSSWDDFSDTDIFSDDEEIAKTMSENDDTLRSLVKMGYKQVEALIAIERLGPNASLEELVDFIGVAQMAKAEDALLPPQEKLQYNDYAKSNKRRLYDYEVLGRKKPRGCEKKILNEDDEDAEALHLPNPMIGFGVPTESSFITHRRLPEDAIGPPYFYYENVALAPKGVWQTISRFLYDVEPEFVDSKFFCAAARKRGYIHNLPIQNRFPLLPLPPRTIHEAFPLTKKWWPSWDIRTKLNCLQTCIGSAKLTERIRKAVEIYDEDPPESVQKYVLHQCRKWNLVWVGRNKVAPLEPDEVETLLGFPRNHTRGGGISRTDRYKSLGNSFQVDTVAYHLSVLKEMYPNGINLLSLFSGIGGAEVALHRLGIPLKNVVSVEKSEVNRNIVRSWWEQTNQKGNLYDMDDVRELDGDRLEQLMSTFGGFDLIVGGSPCNNLAGSNRVSRDGLEGKESSLFFDYFRILDLVKNMSAKYR

SEQ ID NO: 17 - DNA, 552 bp, Artificial Sequence
  Generalized N19 ATU3B gene for expressing a S Pyogenes sgRNA
tttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcannnnnnnnnnnnnnnnnnngttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaattt

SEQ ID NO: 18 - DNA, 1142 bp, Artificial Sequence
  Tandem ATU3B gene cassette for expressing two S Pyogenes type sgRNAs targeting CCA1
aattgaattccctgcaggaatctttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcattcagtttcccctcttttgttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaatttcggatttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcaccgagaaaatttaatcgcgttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaattttactggtaccatat

SEQ ID NO: 19 - DNA, 18632 bp, Artificial Sequence
  binary plasmid containing a XVE regulated DCAS9-SoyDRM2 DNA methyltransferase fusion protein
gaattcatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctctcgaccaggtaaatttctagtttttctccttcattttcttggttaggacccttttctctttttatttttttgagctttgatctttctttaaactgatctattttttaattgattggttatggcgcaaatattacatagctttaactgataatctgattactttatttcgtgtgtctatgatgatgatgatgttacaggtctcgacgaagctagtcaccatggctatgaaggccctgaccgccaggcagcaggaggtgttcgacctgatcagggaccacatctcccagaccggcatgccaccgaccagggccgagatcgcccagaggctgggcttcaggtccccgaacgctgccgaggagcacctgaaggccctggccaggaagggcgtgatcgagatcgtgtccggagcctccaggggcatcaggctgctgcaagaggaggaggagggcctgccgctggtgggcagggtggctgctggcgagccgtcctccgctccgccgaccgacgtgtccctgggcgacgagctgcacctggacggcgaggacgtggccatggcccacgccgacgccctggacgacttcgacctggacatgctgggcgacggcgactccccaggccctggcttcaccccgcacgactccgctccgtacggtgccctggacatggccgacttcgagttcgagcagatgttcaccgacgccctgggcatcgacgaggctagcatgaggccggagtgcgtggtgccggagacccagtgcgccatgaagaggaaggagaagaaggcccagaaggagaaggacaagctgccggtgtccaccaccaccgtggacgaccacatgccaccgatcatgcaatgcgagccgccacctccggaggctgcccgcatccacgaggtggtgccgaggttcctgtccgacaagctgctggagaccaacaggcagaagaacatcccgcagctgaccgccaaccagcagttcctgatcgccaggctgatctggtatcaggacggctacgagcagccgtccgacgaggacctgaagaggatcacccagacctggcagcaggccgacgacgagaacgaggagtccgacaccccgttcaggcagatcaccgagatgaccatcctgaccgtgcaactgatcgtggagttcgccaagggcctgccaggcttcgccaagatctcccagccggaccagatcaccctgctgaaggcctgctcctccgaggtgatgatgctgagggtggccaggaggtacgacgctgcctccgactccgtgctgttcgccaacaaccaggcctacaccagggacaactaccggaaggctggcatggcctacgtgatcgaggacctgctgcacttctgccggtgcatgtactccatggccctggacaacatccactacgccctgctgaccgccgtggtgatcttctccgacaggccaggcctggagcagccgcagctggtggaggagatccagaggtactacctgaacaccctgaggatctacatcctgaaccagctgtccggctccgccaggtcctccgtgatctacggcaagatcctgtccatcctgtccgagctgaggaccctgggcatgcaaaactccaacatgtgcatctccctgaagctgaagaacaggaagctgccaccgttcctggaggagatctgggacgtggccgacatgtcccacacccagccaccgccgatcctggagtccccgaccaacctgtgagagctctgtccaacagtctcagggttaatgtctatgtatcttaaataatgttgtcggcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactggatccagcttgggctgcaggtcgaggctaaaaaactaatcgcattatcatcccctcgacgtactgtacatataaccactggttttatatacagcagtactgtacatataaccactggttttatatacagcagtcgacgtactgtacatataaccactggttttatatacagcagtactgtacatataaccactggttttatatacagcagtcgaggtaagattagatatggatatgtatatggatatgtatatggtggtaatgccatgtaatatgctcgactctaggatcttcgcaagacccttcctctatataaggaagttcatttcatttggagaggacacgctgaagctagtcctcgagaccaccatgccaaagaagaagcggaaggtagaccctaagaagaagcgcaaggtcgacggcagcgggagcatggacaagaagtacagcatcggcctggccatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctccaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctgtacctgtactacctgcagaacggccgcgacatgtacgtggaccaggagctggacatcaaccgcctgtccgactacgacgtggacgccatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgacggcggaagctctagaatgggaggagatgattctggtttggagagtgacaattttgattggaacactgaagatgagcttgaaattcagaactataactcgtcgtcttcatgtttaacccttcctaatggagatgctgttactggctctggagaggcaagctcgtctgcagttttggctaattccaaggtgcttgatcacttcgtcagcatgggatttagcagagaaatggtttctaaagtaattcaggaatatggtgaggaaaatgaagataaactacttgaagaacttctcacatacaaggccctagaaagttcttcccgtccacagcagcgaattgagccagatccttgttcttcagagaatgcagggagttcttgggatgatttctcagatactgatattttttctgatgatgaagaaattgcaaaaactatgtctgagaatgatgataccttacggtctttggtgaaaatggggtacaagcaggtggaggctttaattgccatagaaagattaggcccaaacgcctcacttgaagaattggtagattttataggtgttgctcaaatggcaaaggctgaagatgctcttctgcctcctcaagaaaagttacaatacaatgactatgctaagtccaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctggacttggtaaaaaatatgtcggccaaatatcgatgatttcccgggctgctttaatgagatatgcgagacgcctatgatcgcatgatatttgctttcaattctgttgtgcacgttgtaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttcctgcaggaatctttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcattcagtttcccctcttttgttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaatttcggatttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcaccgagaaaatttaatcgcgttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaattttactggtaccataaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcatcagatttcggtgacgggcaggaccggacggggcggtactggcaggctgaagtccagctgccagaaacccacgtcatgccagttcccgtgcttgaagccggccgcccgcagcatgccgcgtggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgattcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagatcctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 20 - DNA, 1144 bp, Artificial Sequence
  Tandem U3 genes for expressing two sgRNAs targetting LHY
aattgaattccctgcaggaatctttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcaaaaaataggaaaagtgcaagttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaatttcggatttactttaaatttttcttatggctcagcctgtgatggataactgaatcaaacaaatggcgtctgggtttaagaagatctgttttggctatgttggacgaaacaagtgaacttttaggatcaacttccgtttatatacggagcttatatcgagcaataagataagtgggctttttatgtaatttaatgggctatcgtccatatattcactaatacccatgcccagtacccatgtatgcgtttcatataagctcctaatttctcccacatcgctcaaatctaaacaaatcttgttgtatatataacactgagggagcaccattggtcaaatccacacgaaattgtaggttttagagctagaaatagcaagttaaaataaggctagtccgttatcaacttgaaaaagtggcaccgagtcggtgctttccttttttcttttttttgccataaacttaaatttgtatatcgatcattgtagatattgaaaacctagaacaaaccaacatccatgtgaatgtctttcatgactgatttagagataattcttgaattttactggtaccatat

SEQ ID NO: 21 - DNA, 1172 bp, Glycine max
  5′UTR (1)..(10)
  CDS (11)..(1162): coding region of catalytic domain of soybean DRM2, lacking an ATG as it will be fused downstream of a DNA binding domain.
atattctagaaataaacgaagattatatgattatgaagtgctgggaaggaaaaagcctaggggatgtgagaagaaaatcctcaatgaagatgacgaggatgctgaagctcttcatcttccaaatccgatgattgggtttggtgtacctacagagtcaagcttcataacacacagaaggcttccagaagatgccattgggcctccttacttctattacgagaatgtagccttagcaccaaaaggtgtttggcaaacaatttcaagattcttgtatgatgttgaacctgagtttgtagattcgaagtttttctgtgctgcagccaggaaaaggggatatattcacaatctccccatccaaaataggttcccacttctaccacttccaccgcgcacaatacatgaggcttttcccctaacaaagaaatggtggccctcatgggatattaggaccaagcttaattgtttgcaaacatgtattggcagtgcaaaactaacagaaagaattaggaaagctgtagaaatctatgatgaagatccacctgaaagtgtacagaagtatgttcttcatcagtgtaggaaatggaatttggtttgggtgggaagaaataaggttgctccattagagcctgatgaagtagaaacgctgttgggcttcccaaggaaccacaccagaggaggtgggataagtaggactgacagatacaagtcacttggtaattcattccaggttgacactgtggcataccacttgtcagttctgaaggagatgtatcctaatggtatcaatcttctttctctcttttctggaattggtggggcggaggtagctcttcatcggcttggcatccctctcaagaatgttgtgtctgttgaaaaatcggaagttaataggaatattgttagaagttggtgggagcaaacaaaccagaaaggtaatctatatgatatggatgatgtaagggagctagacggtgatcgcttggaacagctgatgagcacatttggtggttttgatctaattgttggtggcagtccatgtaataacctggctggtagcaacagggtcagccgggatggactggagggaaaagaatcttctctcttctttgattattttaggattctggacttggtaaaaaatatgtcggccaaatatcgatgacccgggatat

SEQ ID NO: 22 - protein, 383 aa, Glycine max
NKRRLYDYEVLGRKKPRGCEKKILNEDDEDAEALHLPNPMIGFGVPTESSFITHRRLPEDAIGPPYFYYENVALAPKGVWQTISRFLYDVEPEFVDSKFFCAAARKRGYIHNLPIQNRFPLLPLPPRTIHEAFPLTKKWWPSWDIRTKLNCLQTCIGSAKLTERIRKAVEIYDEDPPESVQKYVLHQCRKWNLVWVGRNKVAPLEPDEVETLLGFPRNHTRGGGISRTDRYKSLGNSFQVDTVAYHLSVLKEMYPNGINLLSLFSGIGGAEVALHRLGIPLKNVVSVEKSEVNRNIVRSWWEQTNQKGNLYDMDDVRELDGDRLEQLMSTFGGFDLIVGGSPCNNLAGSNRVSRDGLEGKESSLFFDYFRILDLVKNMSAKYR

SEQ ID NO: 23 - DNA, 3380 bp, Glycine max
  5′UTR (1)..(10)
  CDS (11)..(3367): full length soybean CMT2 CDS
atattctagaatgaaatcgccgcagcacaaaaacaaaacaccaaaatcccgatcagatcctcaacaacaacaaaaatccaattccctggccgtcaccgttttccgaaatgcacccgattattattcctgcgacccccttattccgttaagcacgtgcctccccgcctcgggcacgtgcgccgccagaatgcgcctactgccgcatgcagatcaggctctcagtctccgccgctccccccgcttcaattctgctccggccgccgtggatggcggcaatgcgggctccagaaagcggaagcggtcgccggaaacggcggcgacggcagcggaggaggagacatctcggagacgctccccgagattctcaacacccgttaagtggcaagggtctaggggtccagaattaccattgaatgcttacctaaaggaccaaaatgtagattccaatcttcattcagatgctaagcctatcaaaaattctcacgggaagttctttggtgagaagcacttgaggcagtgcccaggattagatccatcatgtgaggcatatgcagaaaagcttgaggtgaaactgcatggtacattggagcctaggagatctcctagactttcatcgtcaaatgggaatgaaaacacggaaattaatgcttcacaagttaaaagaacatcaaaagtagttcaccaaaagaaaaaaaataccaaagtgatttctacttacattgaattgtctaattcaccagttgagcagcatatgtgtggaattgatgatacaaaatacttgataaggtcttcagaggcaatcacatccccatcctggactgaaaatggcagaacaacttctaattcctttgccttgcctgagtcagatgacaggccaccaagaaagaaatttaatacatctacttcttcaactaaggaatgtatagattcagaaaactttccttccttcattggtgatccaatacctgatgatgaagctcagaaaagatggggatggcgttatgaattaaaggacaaaaaatgtaaagacaacatgtttaaaataaacgagggtgaagaagatgagattattgcaaatgtcaaatgccattatgcccaagctgaaattgggaattgtatttttagtcttggcgattgtgcatttgtaaaaggtgaaggagaagaaaaacatgttggcaagattattgaatttttccaaacaactgatggtcaaaattatttccgagtccaatggttttacagaattcaagatacagttgtccaagacgaaggtggttttcatgataagaggcgtgtattttattcagcaataatgaacgataacttaatagattgcataatgggaaaagctaatgtcacacatataacgcctagggttggtctgaagttggcttcaatttcatcatctgacttctattatgacatggaatattgcgtggattattctacattccgcaacatacctacagatgcctctacagtaactgagagtcaaccctgttctgagttgaacaagacagaattagcattactagatctctattctgggtgtggtggcatgtcaactggattgtgcttaggtgccaaaactgcatcagttaaccttgtcacgagatgggctgttgatagtgataggtctgctggtgaaagcttgaaactaaaccattcggacacccatgtgcgaaatgaatctgccgaggactttctggaactattgaaggcgtgggaaaagctatgcaaacggtataatgtcagcagcacagaaagaaaacttccatttcgatccaattcttcaggagcaaagaaacgagggaattctgaagttcacgaaatttctgatggtgaacttgaagtttctaagttagttgatatttgttttggtgatcccaatgaaactggcaaacgtggtctatatttgaaggtgcattggaagggttacagtgcaagtgaagacacatgggaaccaattaaaagcttaagcaagtgtaaggaaagcatgcaggattttgtgaggaaagggatgaaatcaaatatcttgcctcttcctggtgaagttgatgtcatttgtgggggtcctccttgtcaaggaataagtggatataatcgctttcgaaactgtgcatcaccattagatgatgaaaggaatcgtcagattgtgattttcatggacatggtgaaatttttgaagcctaggtatgttttaatggaaaatgtggttgacatattgagatttgacaagggttcgcttggaagatatgcattgagtcgtttggtacatatgaactaccaagcaaggttgggaatcattgctgctgggtgttatggtcttccccaatttcggttgcgtgttttcttgtggggtgctcatccaagtgaggttataccccaatttccactgccaacgcatgatgttatcgtcagatactggcctccaccagagtttgagcgcaatgttgttgcttatgatgaagagcaacctcgtgagttagagaaagctactgttattcaagatgccatctctgatcttcctgctgtcatgaacactgaaactcgtgatgaaatgccatatcaaaatcctccagaaacagaatttcaaagatatatacgatcaactaagtatgagatgactgggtcaaaatcaaatggcacaacagagaaaagacccttattgtatgatcaccgtccctactttttgtttgaagatgactacctacgtgtttgtcaaattccaaaacgcaagggcgcaaatttccgtgatctgcctggtgtaattgtaggagctgacaatgtggttagacgacatccgacagagaatccattgctaccatctggaaagccattggtcccagaatattgcttcacttttgagcatgggaagtctaagagaccatttgcacggttgtggtgggacgagaatcttcctactgcactaaccttcccaagttgtcataaccaggttgtactacatccggagcaggatcgagtcctcactatacgagaatttgccaggctgcaaggatttcctgattattatagattctatgggactgtaaaagagaggtactgtcaaattggaaatgcagttgctgttcctgtttcacgggctttaggttatgccctaggacttgcttgtagaaagctcaatggaaatgagccacttgtaaccctcccgtccaagttctctcactccaattatcttcagttatctaaatgcgtgtttggaaacacgtccaacgaggtgaattcacgtcaattcagggctttggacgcggaagttacaccgggtagcattggccaagactcacgtgtggaagattcgacacagctccaaacatgctataataatcagcctggcaacactgactaaatacccgggatat

SEQ ID NO: 24 - protein, 1118 aa, Glycine max
MKSPQHKNKTPKSRSDPQQQQKSNSLAVTVFRNAPDYYSCDPLIPLSTCLPASGTCAARMRLLPHADQALSLRRSPRFNSAPAAVDGGNAGSRKRKRSPETAATAAEEETSRRRSPRFSTPVKWQGSRGPELPLNAYLKDQNVDSNLHSDAKPIKNSHGKFFGEKHLRQCPGLDPSCEAYAEKLEVKLHGTLEPRRSPRLSSSNGNENTEINASQVKRTSKVVHQKKKNTKVISTYIELSNSPVEQHMCGIDDTKYLIRSSEAITSPSWTENGRTTSNSFALPESDDRPPRKKFNTSTSSTKECIDSENFPSFIGDPIPDDEAQKRWGWRYELKDKKCKDNMFKINEGEEDEIIANVKCHYAQAEIGNCIFSLGDCAFVKGEGEEKHVGKIIEFFQTTDGQNYFRVQWFYRIQDTVVQDEGGFHDKRRVFYSAIMNDNLIDCIMGKANVTHITPRVGLKLASISSSDFYYDMEYCVDYSTFRNIPTDASTVTESQPCSELNKTELALLDLYSGCGGMSTGLCLGAKTASVNLVTRWAVDSDRSAGESLKLNHSDTHVRNESAEDFLELLKAWEKLCKRYNVSSTERKLPFRSNSSGAKKRGNSEVHEISDGELEVSKLVDICFGDPNETGKRGLYLKVHWKGYSASEDTWEPIKSLSKCKESMQDFVRKGMKSNILPLPGEVDVICGGPPCQGISGYNRFRNCASPLDDERNRQIVIFMDMVKFLKPRYVLMENVVDILRFDKGSLGRYALSRLVHMNYQARLGIIAAGCYGLPQFRLRVFLWGAHPSEVIPQFPLPTHDVIVRYWPPPEFERNVVAYDEEQPRELEKATVIQDAISDLPAVMNTETRDEMPYQNPPETEFQRYIRSTKYEMTGSKSNGTTEKRPLLYDHRPYFLFEDDYLRVCQIPKRKGANFRDLPGVIVGADNVVRRHPTENPLLPSGKPLVPEYCFTFEHGKSKRPFARLWWDENLPTALTFPSCHNQVVLHPEQDRVLTIREFARLQGFPDYYRFYGTVKERYCQIGNAVAVPVSRALGYALGLACRKLNGNEPLVTLPSKFSHSNYLQLSKCVFGNTSNEVNSRQFRALDAEVTPGSIGQDSRVEDSTQLQTCYNNQPGNTD

SEQ ID NO: 25 - DNA, 1967 bp, Glycine max
  5′UTR (1)..(10)
  CDS (11)..(1954): catalytic domain of soybean CMT2 and lacking an ATG as it is meant to be used as a fusion domain downstream of a DNA binding domain.
atattctagaatggaatattgcgtggattattctacattccgcaacatacctacagatgcctctacagtaactgagagtcaaccctgttctgagttgaacaagacagaattagcattactagatctctattctgggtgtggtggcatgtcaactggattgtgcttaggtgccaaaactgcatcagttaaccttgtcacgagatgggctgttgatagtgataggtctgctggtgaaagcttgaaactaaaccattcggacacccatgtgcgaaatgaatctgccgaggactttctggaactattgaaggcgtgggaaaagctatgcaaacggtataatgtcagcagcacagaaagaaaacttccatttcgatccaattcttcaggagcaaagaaacgagggaattctgaagttcacgaaatttctgatggtgaacttgaagtttctaagttagttgatatttgttttggtgatcccaatgaaactggcaaacgtggtctatatttgaaggtgcattggaagggttacagtgcaagtgaagacacatgggaaccaattaaaagcttaagcaagtgtaaggaaagcatgcaggattttgtgaggaaagggatgaaatcaaatatcttgcctcttcctggtgaagttgatgtcatttgtgggggtcctccttgtcaaggaataagtggatataatcgctttcgaaactgtgcatcaccattagatgatgaaaggaatcgtcagattgtgattttcatggacatggtgaaatttttgaagcctaggtatgttttaatggaaaatgtggttgacatattgagatttgacaagggttcgcttggaagatatgcattgagtcgtttggtacatatgaactaccaagcaaggttgggaatcattgctgctgggtgttatggtcttccccaatttcggttgcgtgttttcttgtggggtgctcatccaagtgaggttataccccaatttccactgccaacgcatgatgttatcgtcagatactggcctccaccagagtttgagcgcaatgttgttgcttatgatgaagagcaacctcgtgagttagagaaagctactgttattcaagatgccatctctgatcttcctgctgtcatgaacactgaaactcgtgatgaaatgccatatcaaaatcctccagaaacagaatttcaaagatatatacgatcaactaagtatgagatgactgggtcaaaatcaaatggcacaacagagaaaagacccttattgtatgatcaccgtccctactttttgtttgaagatgactacctacgtgtttgtcaaattccaaaacgcaagggcgcaaatttccgtgatctgcctggtgtaattgtaggagctgacaatgtggttagacgacatccgacagagaatccattgctaccatctggaaagccattggtcccagaatattgcttcacttttgagcatgggaagtctaagagaccatttgcacggttgtggtgggacgagaatcttcctactgcactaaccttcccaagttgtcataaccaggttgtactacatccggagcaggatcgagtcctcactatacgagaatttgccaggctgcaaggatttcctgattattatagattctatgggactgtaaaagagaggtactgtcaaattggaaatgcagttgctgttcctgtttcacgggctttaggttatgccctaggacttgcttgtagaaagctcaatggaaatgagccacttgtaaccctcccgtccaagttctctcactccaattatcttcagttatctaaatgcgtgtttggaaacacgtccaacgaggtgaattcacgtcaattcagggctttggacgcggaagttacaccgggtagcattggccaagactcacgtgtggaagattcgacacagctccaaacatgctataataatcagcctggcaacactgactaaatacccgggaatt

SEQ ID NO: 26 - protein, 647 aa, Glycine max
MEYCVDYSTFRNIPTDASTVTESQPCSELNKTELALLDLYSGCGGMSTGLCLGAKTASVNLVTRWAVDSDRSAGESLKLNHSDTHVRNESAEDFLELLKAWEKLCKRYNVSSTERKLPFRSNSSGAKKRGNSEVHEISDGELEVSKLVDICFGDPNETGKRGLYLKVHWKGYSASEDTWEPIKSLSKCKESMQDFVRKGMKSNILPLPGEVDVICGGPPCQGISGYNRFRNCASPLDDERNRQIVIFMDMVKFLKPRYVLMENVVDILRFDKGSLGRYALSRLVHMNYQARLGIIAAGCYGLPQFRLRVFLWGAHPSEVIPQFPLPTHDVIVRYWPPPEFERNVVAYDEEQPRELEKATVIQDAISDLPAVMNTETRDEMPYQNPPETEFQRYIRSTKYEMTGSKSNGTTEKRPLLYDHRPYFLFEDDYLRVCQIPKRKGANFRDLPGVIVGADNVVRRHPTENPLLPSGKPLVPEYCFTFEHGKSKRPFARLWWDENLPTALTFPSCHNQVVLHPEQDRVLTIREFARLQGFPDYYRFYGTVKERYCQIGNAVAVPVSRALGYALGLACRKLNGNEPLVTLPSKFSHSNYLQLSKCVFGNTSNEVNSRQFRALDAEVTPGSIGQDSRVEDSTQLQTCYNNQPGNTD

SEQ ID NO: 27 - DNA, 2651 bp, Glycine max
  5′UTR (1)..(10)
  CDS (11)..(2638): Full length CDS of soybean CMT3
atattctagaatgcctagcaagcgcaagaccagatcctccgcctctcccgctgccgcgccgccgagcaagcgcgcctccagatcctccgcctctcgcgtcgccgattccgcaccggtcaaatctgaagccgaggaagttgtggcagcttcctctgtcgtcaaagaagaggcgcaagcaagcttcacggacgttactgacggcaacgttagcgatggcgaggggactaacgccagattcgtcggagagcctgttcccgacgaggaagcccggcgccgctggccgaaacgctaccaggaaaaggaaaagaagcaatccgctgggccaaaatcaaacagaaacgatgaagacgaggagattcaacaagctcgacgtcactacactcaggcagaagtagatgggtgcatgctttacaaactttacgatgatgcccatgttaaagcagaagaaggagaagacaattacatttgtaaaattgttgagatatttgaggccattgatggggcactgtattttacggcacaatggtattatagggctaaagacactgtcattaaaaaacttgcatatctcattgaaccaaagcgagttttcttttctgaagtccaggatgacaaccctttggattgtctagttgaaaagctgaacatcgccagaataacattaaatgtagatttagaagcaaagaaggaaaccattccaccttgtgattattactgtgatacacaatatcttttgccatactccacatttgttaacttaccatcagaaaatggggaatctggtagtgaaacttcttctacaatatcttctgaaactaatggaatcggaaaatatgaggtgaactctcaacctaaggaagcttttcttcccgaagaaagtaaagatccggagatgaagttactagatttatattgtggttgtggtgcaatgtcaactggtttgtgccttggtggaaatttatctggtgtgaaccttgttactagatgggcagtggacttgaatcaacatgcttgtgaatgtcttaaattaaaccatcctgaaactgaggttagaaatgaatcggcagaaaattttctttcattattgaaggagtggcaggaattatgtagttacttctctctagttgaaaaaaaggtgtcacatgagaaatatgtgaatctttttagtgaagatgacgatgacactagcagtaatgaagaggttaatagtgaagatgacaatgaactgaatgaagatgatgaaatatttgaagtttctgaaatccttgctgtctgctacggtgacccaaataagaaaaaagaacaagggttatacttcaaggttcattggaagggttatgaatctgccctggattcttgggaaccaattgaaggtctaagtaattgcaaggaaaagattaaagaatttgtcagtcgaggcttcaagtcacagatattgcctttgcctggagatgttgatgtaatttgtggtggacctccttgccaaggtattagtggtttcaaccggtttcggaacaaagagagtcctttggatgatgagaagaacaaacaactagttgtttttatggatattgttcaataccttaagcccaaatttacattgatggaaaatgtggttgatcttgtaaaatttgcggaaggctttcttgggagatatgccttgggtcgccttcttcaaatgaattatcaagcgcgtttaggaattatggctgcaggtgcttatgggcttcctcagtttcgtttgcgcgtctttttatggggggctgcaccttctcagaagttgccacaatttccgcttccaactcatgatgttattgtaaggggtgttattcccttggagtttgagataaacactgtagcatacaatgaaggacaaaaggttcaactgcagaagaagcttttattggaggatgctatttctgaccttcctcgggttcagaacaatgagcgtcgtgatgagataaaatatgacaaagctgctcaaacggagttccaacgattcattagattaagcaaacatgaaatgttggagcttcaatccagaacaaaatcgtcgaagtctttgctatatgatcatcgtccactagaattgaatgcggatgattaccaacgtgtgtgtcggatccctaaaaagaagggtggatgcttcagagatttaccaggtgttcgtgtgggagctgataacaaggttgaatgggatcctgatgtcgaacgtgtatatttggattcaggaaaaccattggttccagattatgccatgacttttgtgaatggaacttcatcaaaaccttttgctcggttatggtgggatgaaactgttccaactgttgtgacaagagcagaacctcacaaccaggcaattttacaccctgaacaagacagagtgttgacgattcgtgaaaatgcaagactccaaggttttccagatttctacaagttgtgtgggccggtcaaagaaaggtacattcaagttgggaatgcagtggcagttccagtagctagagctttaggatacacactaggccttgcatttgaagggtctacttctacaagtgatgatccattgtataaattacctgataaatttcccatgattagggatcgggtttcttctgtatcttccgaagatgatgtgtaaatacccgggaatt

SEQ ID NO: 28 - protein, 875 aa, Glycine max
MPSKRKTRSSASPAAAPPSKRASRSSASRVADSAPVKSEAEEVVAASSVVKEEAQASFTDVTDGNVSDGEGTNARFVGEPVPDEEARRRWPKRYQEKEKKQSAGPKSNRNDEDEEIQQARRHYTQAEVDGCMLYKLYDDAHVKAEEGEDNYICKIVEIFEAIDGALYFTAQWYYRAKDTVIKKLAYLIEPKRVFFSEVQDDNPLDCLVEKLNIARITLNVDLEAKKETIPPCDYYCDTQYLLPYSTFVNLPSENGESGSETSSTISSETNGIGKYEVNSQPKEAFLPEESKDPEMKLLDLYCGCGAMSTGLCLGGNLSGVNLVTRWAVDLNQHACECLKLNHPETEVRNESAENFLSLLKEWQELCSYFSLVEKKVSHEKYVNLFSEDDDDTSSNEEVNSEDDNELNEDDEIFEVSEILAVCYGDPNKKKEQGLYFKVHWKGYESALDSWEPIEGLSNCKEKIKEFVSRGFKSQILPLPGDVDVICGGPPCQGISGFNRFRNKESPLDDEKNKQLVVFMDIVQYLKPKFTLMENVVDLVKFAEGFLGRYALGRLLQMNYQARLGIMAAGAYGLPQFRLRVFLWGAAPSQKLPQFPLPTHDVIVRGVIPLEFEINTVAYNEGQKVQLQKKLLLEDAISDLPRVQNNERRDEIKYDKAAQTEFQRFIRLSKHEMLELQSRTKSSKSLLYDHRPLELNADDYQRVCRIPKKKGGCFRDLPGVRVGADNKVEWDPDVERVYLDSGKPLVPDYAMTFVNGTSSKPFARLWWDETVPTVVTRAEPHNQAILHPEQDRVLTIRENARLQGFPDFYKLCGPVKERYIQVGNAVAVPVARALGYTLGLAFEGSTSTSDDPLYKLPDKFPMIRDRVSSVSSEDDV

SEQ ID NO: 29 - DNA, 1772 bp, Glycine max
  5′UTR (1)..(10)
  CDS (11)..(1759): Catalytic domain of soybean CMT3
atattctagagagatgaagttactagatttatattgtggttgtggtgcaatgtcaactggtttgtgccttggtggaaatttatctggtgtgaaccttgttactagatgggcagtggacttgaatcaacatgcttgtgaatgtcttaaattaaaccatcctgaaactgaggttagaaatgaatcggcagaaaattttctttcattattgaaggagtggcaggaattatgtagttacttctctctagttgaaaaaaaggtgtcacatgagaaatatgtgaatctttttagtgaagatgacgatgacactagcagtaatgaagaggttaatagtgaagatgacaatgaactgaatgaagatgatgaaatatttgaagtttctgaaatccttgctgtctgctacggtgacccaaataagaaaaaagaacaagggttatacttcaaggttcattggaagggttatgaatctgccctggattcttgggaaccaattgaaggtctaagtaattgcaaggaaaagattaaagaatttgtcagtcgaggcttcaagtcacagatattgcctttgcctggagatgttgatgtaatttgtggtggacctccttgccaaggtattagtggtttcaaccggtttcggaacaaagagagtcctttggatgatgagaagaacaaacaactagttgtttttatggatattgttcaataccttaagcccaaatttacattgatggaaaatgtggttgatcttgtaaaatttgcggaaggctttcttgggagatatgccttgggtcgccttcttcaaatgaattatcaagcgcgtttaggaattatggctgcaggtgcttatgggcttcctcagtttcgtttgcgcgtctttttatggggggctgcaccttctcagaagttgccacaatttccgcttccaactcatgatgttattgtaaggggtgttattcccttggagtttgagataaacactgtagcatacaatgaaggacaaaaggttcaactgcagaagaagcttttattggaggatgctatttctgaccttcctcgggttcagaacaatgagcgtcgtgatgagataaaatatgacaaagctgctcaaacggagttccaacgattcattagattaagcaaacatgaaatgttggagcttcaatccagaacaaaatcgtcgaagtctttgctatatgatcatcgtccactagaattgaatgcggatgattaccaacgtgtgtgtcggatccctaaaaagaagggtggatgcttcagagatttaccaggtgttcgtgtgggagctgataacaaggttgaatgggatcctgatgtcgaacgtgtatatttggattcaggaaaaccattggttccagattatgccatgacttttgtgaatggaacttcatcaaaaccttttgctcggttatggtgggatgaaactgttccaactgttgtgacaagagcagaacctcacaaccaggcaattttacaccctgaacaagacagagtgttgacgattcgtgaaaatgcaagactccaaggttttccagatttctacaagttgtgtgggccggtcaaagaaaggtacattcaagttgggaatgcagtggcagttccagtagctagagctttaggatacacactaggccttgcatttgaagggtctacttctacaagtgatgatccattgtataaattacctgataaatttcccatgattagggatcgggtttcttctgtatcttccgaagatgatgtgtaaatacccgggaatt

SEQ ID NO: 30 - protein, 582 aa, Glycine max
EMKLLDLYCGCGAMSTGLCLGGNLSGVNLVTRWAVDLNQHACECLKLNHPETEVRNESAENFLSLLKEWQELCSYFSLVEKKVSHEKYVNLFSEDDDDTSSNEEVNSEDDNELNEDDEIFEVSEILAVCYGDPNKKKEQGLYFKVHWKGYESALDSWEPIEGLSNCKEKIKEFVSRGFKSQILPLPGDVDVICGGPPCQGISGFNRFRNKESPLDDEKNKQLVVFMDIVQYLKPKFTLMENVVDLVKFAEGFLGRYALGRLLQMNYQARLGIMAAGAYGLPQFRLRVFLWGAAPSQKLPQFPLPTHDVIVRGVIPLEFEINTVAYNEGQKVQLQKKLLLEDAISDLPRVQNNERRDEIKYDKAAQTEFQRFIRLSKHEMLELQSRTKSSKSLLYDHRPLELNADDYQRVCRIPKKKGGCFRDLPGVRVGADNKVEWDPDVERVYLDSGKPLVPDYAMTFVNGTSSKPFARLWWDETVPTVVTRAEPHNQAILHPEQDRVLTIRENARLQGFPDFYKLCGPVKERYIQVGNAVAVPVARALGYTLGLAFEGSTSTSDDPLYKLPDKFPMIRDRVSSVSSEDDV

SEQ ID NO: 31 - DNA, 4511 bp, Glycine max
  5′UTR (1)..(10)
  CDS (11)..(4498): Full length CDS of soybean Met1
atattctagaatgccaaaacgtgctgcggcatgtaaaaatttgaaggagaaatcctttttgatatatgagaagtcctgcctcattgaaacagagaaggatcatattgtagaagaagaaagtctggctgtccgcatgacagctggacaggataatggttgtccaaataggcgcattacagagtttatccttcatgatgaaactggtaaatcccagccacttgaggtgctggaggttgatgatttgtttatcactggacttgtattgccacttgaagccagctctggcaagaaaaaagagaaaggtgttaagtgtgaaggctttggtcgaattgaatcatgggatatatctggttatgaagatggctctccagtgatatggctttctactgaagttgctgattatgattgccagaaacctgctgctagttacaaaaaagtttacgatcttttccttgaaaaggcccgtgcatgtgtagaagtatacaagaaacttgcaaagtcctctggtggtgaccctgatataagtcttgatgagttacttgctggcatggtgcggtctatgagtggtagcaaatgcttttctggagctgcatctataaaggattttgttatttcacagggtgagttcatttataagcaacttgttggtttggatatgacatccaaggcaaatgacaggatgtttgcagatattcctgctcttattgctcttagagatgagagtaagaaacaagtacatgcacaggtaatgccctcaaatgggagtttaaggattgattcaggagttggagatgaagaaaacaagaatcagatggattcagtagcttctgtaaacgaggaagatgaggatgcaaagctggcacggctgttgcaggaagaagagtattggcaatctatgaaccagaagaaaaactccagatcagcctctgcatcgaacaaatactatatcaaaattaatgaagatgagattgccaatgattatcctctacctgtttattataaaacctcccttcaagaaacagatgagtttatagtttttgataatgactatgacatatatgacactcaagatctccctcgaagcatgctgcataattggtctttatacaactcagatgcaagattggtttccttggaacttctgcctatgaaaccttgttcagatatcgatgttgcaatctttggatcaggtataatgacttcagatgatggaagtgggtttcatcttgatactgaggctggcaaatcttcttccgttggttctggagcacaggttgctgatggaatgccaatttatctgagtgccataaaggaatggatgattgaatttggatcatctatgattttcatatccatccgaactgatttggcctggtatagacttggcaaaccagcaaagcagtatgctccttggtatgacacagtattgaaaactgcaaggcttgctataagcattatcaccttgctgaaggagcagagccgagtatcacgactttcctttggagatgtcatcaggaaagtatctgagtttgataagaaagacggttcttacatttcttctgatccattgactgttgagagatatgttgttgtccatggacagataattctgcaactgtttgcagaatttcctgatgataagatcagaaagtctgcatttgtgacgggtcttacaaacaaaatggaagagcgccaccataccaaatggttggtgaagaagaagaaagttgtgccaaggagtgaaccaaatttaaatcctagagcagcagtgggtcctgttgtatccaagaggaaagctatgcaagctacaacgacaaggctgatcaatagaatatggggagagtattactcaaaccacttgccagaggatgcaaaagagggaattgctagtgagttaaaggatgaggatgaagtggaggaacaagaagaaaatgaagatgatgacaatgaggagacaatacttttggagggaaccccaaaggcacattcagcttcaaagcaaaccaaaaaattttctgctgaaacagaaataaggtgggaaggggaacctgaagggaagactagttctggatatcctgtttataaacaggcaattattcgtggggaagttatttctgtaggaagatctgtgttggtggaggttgatgaaacagatgaatttccagacatatattatgttgaatatatgtttgaatcaaagatcggaagaaaaatgttccatggtaggatgatgcagcgtggttgtcagactgttcttggcaatgctgctaatgagagagaggtgtttttgactaatgagtgcagggatttgggactgcatgatgtcaatcagacagttgttgtaaatattcaaaataggccttggggacatcagcatcgaaaggataatatcattgcagatagagttgacagggctcaagcagaagaaaggaagaagaaaggactacctactgaatattactgtaaaagcctgtactggcctgaaagaggtgctttctttagccttccacttgatactttggggctagggtctggtgtttgcccatcttgcaaaatacaggatgctgaaaaggagaaggatgttttcaaagtaaattcctccaagtctggtttcctattgaaaggaactgagtattctcttaatgattatatttatgtaagtcccttcgaatttgaggaaatgatagagcaaggaactcataagagtgggaggaatgtggggttgaaagcttatgttgtgtgccaagtgcttgagattgttgtcaaaaaggaaattaaagaagctgaaataaagtctacacaagtcaaaatcaggaggttcttccgaccagaagatgtatcaaatgagaaggcatactgttctgatatacaagaggtgtattacagtgatgaaacacatataatctctgtagaatccatagaagggaaatgtcaagtcagaaaaaagaatgatattcccgaatgcagtgcccttggcagaatgttccaaaatgtttttttctgcgagctcttgtatgatcctgccactgggtcactgaagaagttgccggctcatgtaaaagtaaaatattcaagtggacaaacatctgatgctgcagctagaaagaggaaaggaaaatgtatagagggagatgatgttttagagtccccaaatgaaggaaaaacattaaatgaaaaacgtttagcaaccttggacatttttgcaggttgtggtggcttatcagaggggttgcagcagtcaggagtttcatcaactaaatgggctattgagtatgaagaacctgctggggatgcatttaaagctaatcatcctgaggcattggtgtttattaacaattgcaatgttattcttagggctgtaatggagaagtgtggggacacagatgattgtatctcaacatccgaagctgcagaattggctgcaaagcttgatgagaaggaaataagtagtttaccaatgcctggacaagttgatttcatcaatggtggtcctccatgtcagggtttctctgggatgaataggtttaaccagagcagttggagtaaagtccagtgtgagatgatattggcattcttatcctttgccgattatttccggccaaggtatttcttgttggagaatgtgaggaactttgtgtctttcaataaagggcagacattccgtttaactttggcttcacttcttgagatgggctatcaggtgaggtttggtatccttgaggctggagcatatggggtttcccagtcaagaaaaagggcattcatatgggcagcctctcctgaggatgtgcttcctgaatggcctgaaccaatgcatgtcttttcggcccctgagttgaagattacattatcagaaaatgtccagtatgctgctgtccgcagtactgcaaatggtgctccattacgttcaataactgttcaagatactattggtgatctcccagctgttggcaatggagcctcaaaaggaaacatggagtatcaaaatgatccagtctcatggtttcaaaagaagattcgaggtgatatggttgtcttgactgatcatatatcaaaggagatgaatgaattgaacttgattcgatgccagaaaattcccaagagaccaggcgctgattggcgtgaccttccagaagaaaagataaaattgtctactggacaagttgttgatttgataccatggtgcttgccaaacacggctaagcggcacaatcagtggaagggactgtttggcaggttggattggcaagggaatttcccaacttccattactgaccctcagccaatggggaaggttggaatgtgcttccaccctgaccaagataggattcttactgttcgtgaatgtgctcggtctcaaggcttcccagatagctatcaatttgctggcaatatcatacacaagcaccggcagattggtaatgctgtgcctcctcctctggcatctgcattggggagaaagctcaaggaagcagtggacagtaagagctccacttagatacccgggaatt

SEQ ID NO: 32 - protein, 1495 aa, Glycine max
MPKRAAACKNLKEKSFLIYEKSCLIETEKDHIVEEESLAVRMTAGQDNGCPNRRITEFILHDETGKSQPLEVLEVDDLFITGLVLPLEASSGKKKEKGVKCEGFGRIESWDISGYEDGSPVIWLSTEVADYDCQKPAASYKKVYDLFLEKARACVEVYKKLAKSSGGDPDISLDELLAGMVRSMSGSKCFSGAASIKDFVISQGEFIYKQLVGLDMTSKANDRMFADIPALIALRDESKKQVHAQVMPSNGSLRIDSGVGDEENKNQMDSVASVNEEDEDAKLARLLQEEEYWQSMNQKKNSRSASASNKYYIKINEDEIANDYPLPVYYKTSLQETDEFIVFDNDYDIYDTQDLPRSMLHNWSLYNSDARLVSLELLPMKPCSDIDVAIFGSGIMTSDDGSGFHLDTEAGKSSSVGSGAQVADGMPIYLSAIKEWMIEFGSSMIFISIRTDLAWYRLGKPAKQYAPWYDTVLKTARLAISIITLLKEQSRVSRLSFGDVIRKVSEFDKKDGSYISSDPLTVERYVVVHGQIILQLFAEFPDDKIRKSAFVTGLTNKMEERHHTKWLVKKKKVVPRSEPNLNPRAAVGPVVSKRKAMQATTTRLINRIWGEYYSNHLPEDAKEGIASELKDEDEVEEQEENEDDDNEETILLEGTPKAHSASKQTKKFSAETEIRWEGEPEGKTSSGYPVYKQAIIRGEVISVGRSVLVEVDETDEFPDIYYVEYMFESKIGRKMFHGRMMQRGCQTVLGNAANEREVFLTNECRDLGLHDVNQTVVVNIQNRPWGHQHRKDNIIADRVDRAQAEERKKKGLPTEYYCKSLYWPERGAFFSLPLDTLGLGSGVCPSCKIQDAEKEKDVFKVNSSKSGFLLKGTEYSLNDYIYVSPFEFEEMIEQGTHKSGRNVGLKAYVVCQVLEIVVKKEIKEAEIKSTQVKIRRFFRPEDVSNEKAYCSDIQEVYYSDETHIISVESIEGKCQVRKKNDIPECSALGRMFQNVFFCELLYDPATGSLKKLPAHVKVKYSSGQTSDAAARKRKGKCIEGDDVLESPNEGKTLNEKRLATLDIFAGCGGLSEGLQQSGVSSTKWAIEYEEPAGDAFKANHPEALVFINNCNVILRAVMEKCGDTDDCISTSEAAELAAKLDEKEISSLPMPGQVDFINGGPPCQGFSGMNRFNQSSWSKVQCEMILAFLSFADYFRPRYFLLENVRNFVSFNKGQTFRLTLASLLEMGYQVRFGILEAGAYGVSQSRKRAFIWAASPEDVLPEWPEPMHVFSAPELKITLSENVQYAAVRSTANGAPLRSITVQDTIGDLPAVGNGASKGNMEYQNDPVSWFQKKIRGDMVVLTDHISKEMNELNLIRCQKIPKRPGADWRDLPEEKIKLSTGQVVDLIPWCLPNTAKRHNQWKGLFGRLDWQGNFPTSITDPQPMGKVGMCFHPDQDRILTVRECARSQGFPDSYQFAGNIIHKHRQIGNAVPPPLASALGRKLKEAVDSKSST

SEQ ID NO: 33 - DNA, 1418 bp, Glycine max
  5′UTR (1)..(10)
  CDS (11)..(1405): Catalytic domain of soybean Met1
atattctagaaagaggaaaggaaaatgtatagagggagatgatgttttagagtccccaaatgaaggaaaaacattaaatgaaaaacgtttagcaaccttggacatttttgcaggttgtggtggcttatcagaggggttgcagcagtcaggagtttcatcaactaaatgggctattgagtatgaagaacctgctggggatgcatttaaagctaatcatcctgaggcattggtgtttattaacaattgcaatgttattcttagggctgtaatggagaagtgtggggacacagatgattgtatctcaacatccgaagctgcagaattggctgcaaagcttgatgagaaggaaataagtagtttaccaatgcctggacaagttgatttcatcaatggtggtcctccatgtcagggtttctctgggatgaataggtttaaccagagcagttggagtaaagtccagtgtgagatgatattggcattcttatcctttgccgattatttccggccaaggtatttcttgttggagaatgtgaggaactttgtgtctttcaataaagggcagacattccgtttaactttggcttcacttcttgagatgggctatcaggtgaggtttggtatccttgaggctggagcatatggggtttcccagtcaagaaaaagggcattcatatgggcagcctctcctgaggatgtgcttcctgaatggcctgaaccaatgcatgtcttttcggcccctgagttgaagattacattatcagaaaatgtccagtatgctgctgtccgcagtactgcaaatggtgctccattacgttcaataactgttcaagatactattggtgatctcccagctgttggcaatggagcctcaaaaggaaacatggagtatcaaaatgatccagtctcatggtttcaaaagaagattcgaggtgatatggttgtcttgactgatcatatatcaaaggagatgaatgaattgaacttgattcgatgccagaaaattcccaagagaccaggcgctgattggcgtgaccttccagaagaaaagataaaattgtctactggacaagttgttgatttgataccatggtgcttgccaaacacggctaagcggcacaatcagtggaagggactgtttggcaggttggattggcaagggaatttcccaacttccattactgaccctcagccaatggggaaggttggaatgtgcttccaccctgaccaagataggattcttactgttcgtgaatgtgctcggtctcaaggcttcccagatagctatcaatttgctggcaatatcatacacaagcaccggcagattggtaatgctgtgcctcctcctctggcatctgcattggggagaaagctcaaggaagcagtggacagtaagagctccacttagatacccgggaatt

SEQ ID NO: 34 - protein, 464 aa, Glycine max
KRKGKCIEGDDVLESPNEGKTLNEKRLATLDIFAGCGGLSEGLQQSGVSSTKWAIEYEEPAGDAFKANHPEALVFINNCNVILRAVMEKCGDTDDCISTSEAAELAAKLDEKEISSLPMPGQVDFINGGPPCQGFSGMNRFNQSSWSKVQCEMILAFLSFADYFRPRYFLLENVRNFVSFNKGQTFRLTLASLLEMGYQVRFGILEAGAYGVSQSRKRAFIWAASPEDVLPEWPEPMHVFSAPELKITLSENVQYAAVRSTANGAPLRSITVQDTIGDLPAVGNGASKGNMEYQNDPVSWFQKKIRGDMVVLTDHISKEMNELNLIRCQKIPKRPGADWRDLPEEKIKLSTGQVVDLIPWCLPNTAKRHNQWKGLFGRLDWQGNFPTSITDPQPMGKVGMCFHPDQDRILTVRECARSQGFPDSYQFAGNIIHKHRQIGNAVPPPLASALGRKLKEAVDSKSST

(19) United States
(12) Patent Application Publication
(10) Pub. No.: US 2017/0016017 A1
(43) Pub. Date: Jan. 19, 2017

(54) Method for increasing plant yields

(71) Applicant: Michael E Fromm, Lincoln, NE (US)

(72) Inventor: Michael E Fromm, Lincoln, NE (US)

(21) Appl. No.: 14/806,867

(22) Filed: Jul. 23, 2015

Related U.S. Application Data
(60) Provisional application No. 62/031,692, filed on Jul. 31, 2014.

(51) Int. Cl. C12N 15/82

(52) U.S. Cl. CPC C12N 15/8261

(57) ABSTRACT

The present invention provides methods for obtaining plants that exhibit useful traits by expression of a DNA methyltransferase fusion protein in progenitor plants. Methods for identifying genetic loci that provide for useful traits in plants and plants produced with those loci are also provided. In addition, plants that exhibit the useful traits, parts of the plants including seeds, and products of the plants are provided as well as methods of using the plants. Recombinant DNA vectors and transgenic plants comprising those vectors that express a DNA methyltransferase fusion protein are also provided.

### CROSS-REFERENCE TO RELATED APPLICATIONS

This application claims the benefit of U.S. Provisional Patent Application No. 62/031692, filed Jul. 31, 2014, which is incorporated herein by reference in its entirety.

### INCORPORATION OF SEQUENCE LISTING

The sequence listing contained in the file named “CRISPR_DNA_Methylases_ST25V2.txt”, which is 553,243 bytes in size (measured in operating system MS-Windows), contains 121 sequences, and is contemporaneously filed with this specification by electronic submission (using the United States Patent Office EFS-Web filing system) and is incorporated herein by reference in its entirety. The information recorded in computer readable form is identical to the written sequence listing and drawings submitted in provisional patent application 62/031692, filed Jul. 31, 2014, and the computer readable submission of sequences includes no new matter.

### STATEMENT REGARDING FEDERALLY SPONSORED RESEARCH OR DEVELOPMENT

Not Applicable.

### BACKGROUND OF THE INVENTION

Considerable progress has been made in targeting DNA binding proteins to specific DNA sequences in the genomes of live cells. Zinc fingers, TALENS, and CRISPR/CAS9 proteins or protein/RNA complexes are experimentally amenable to changes in their amino acid sequences or RNA targeting sequences to facilitate their binding to specific DNA sequences (Cai and Yang 2014; Carroll 2014; Gersbach and Perez-Pinera 2014; Kim and Kim 2014). Of these, the most convenient method to target a protein to a specific DNA sequence is with the CRISPR/CAS9 protein/RNA complex (Esvelt, Mali et al, 2013; Hou, Zhang et al. 2013; Fonfara, Le Rhun et al. 2014; Hsu, Lander et al. 2014; Sander and Joung 2014). CR1SPR proteins are members of a large Cas3 class of ['encases found in many prokaryotes [see (Jackson, Lavin et a]. 2014) and references therein], herein referred to as CRISPR/CAS9. CRISPR/CAS9 class of proteins bind either a single guide RNA or two annealed RNAs, that target specific DNA sequences through DNA/RNA complementary base pairing, facilitated by the CRISPR/CAS9 protein unwinding of the DNA (Cai and Yang 2014; Carroll 2014; Gersbach and Perez-Pinera 2014; Kim and Kim 2014). Multiple single guide RNAs (sgRNAs) can be used concurrently, with examples of two (Mao, Zhang et al. 2013), three (Ma, Chang et al. 2014), four (Perez-Pinera, Kocak et al. 2013; Ma, Shen et al. 2014), five (Jao, Wente et al. 2013), six (Liu et al., Insect Biochem Mol Biol. 2014 Jun;49:35-42), or seven (Sakuma, Nishikawa et al. 2014). Most designs utilize repeats of an intact sgRNA gene with its own Pol III U6 or U3 promoter (Sakutna, Nishikawa et al. 2014). A S. pyogenes single guide RNA (sgRNA) has the following design: 20 nucleotide base-pairing region that is complementary or homologous to the target DNA sequence, a 42 nt Cas9 recognition hairpin structure, and a 40 nt S. pyogenes terminator with a 3′ hairpin followed by 4 or more U nt). The general sequence format is: 5′-N20 target- GUUUUAGAGCUAGAAAUAGCAAGUUAAAAUAAGGCUAGUCCGUUAUCAACUUGA AAAAGUGGCACCGAGUCGGUGCUUUUUU-3′ (SEQ ID NO:1). Transcription starts at the N1 position, or a processed transcript that has a 5′ end at the N1 position. Promoters transcribed by RNA Polymerase II can be used to produce sgRNAs due to processing by internal ribozymes at the 5′ and/or 3′ ends of the sgRNA sequences (Gao and Zhao 2014),

The CRISPR/CAS9 system can be used for DNA cleavage, DNA nicking, or binding DNA with a nuclease-inactive form. Mutations in either or both of the nuclease domains in CRISPR/CAS9 or similar type CRISPR proteins allows for binding the DNA without cleaving the DNA (Larson, Gilbert et al. 2013; Qi, Larson et al. 2013). Silencing mutations of the RuvC1 and HNH nuclease domains (D10A and H841A, respectively) are useful for a catalytically inactive CRISPR/CAS9 protein nuclease that is still competent for DNA binding in the presence of one or more sgRNAs (Perez-Pinera, Kocak et al. 2013), Predictive software for useful sgRNA designs is available (Bae, Park et al. 2014; Kunne, Swans et al. 2014; Xiao, Cheng et a . 2014; Xie, Zhang et al. 2014) and progress on the mechanisms of CRISPR DNA recognition is proceeding.

Sequence specific DNA binding proteins such as zinc fingers, TALENS, and CRISPR proteins are useful in plants as well (Bellhaj, Chaparro-Garcia et al. 2013; Shan, Wang et al. 2013; Chen and Gao 2014; Fichtner, Urrea Castellanos et al. 2014; Liu and Fan 2014; Lozano-Juste and Cutler 2014; Puchta and Fauser 2014), Recent publications use catalytically active nucleases in Arabidopsis (Jiang, Zhou et al. 2013; Fauser, Schiml et al. 2014; Feng, Mao et al. 2014; Gao and Zhao 2014; Jiang, Yang et al. 2014); or a nickase in Arabidopsis (Fauser, Schiml et al. 2014); maize (Liang, Zhang et al, 2014); rice (Jiang. Zhou et al. 2013; Miao, Guo et al. 2013; Xu, Li et al. 2014; Zhang, Zhan,s7, et al. 2014); or Wheat (Shan, Wang et al. 2013). (Sternberg, Redding et al. 2014). Singel guide RNAs are typically expressed from U6 or U3 promoters in plants_{;} such as the wheat U6 promoter (Shan, Wang et al. 2013); the rice U3 promoter (Shan, Wang et al. 2013); the maize U3 promoter (Liang, Zhang et al. 2014); or the Arabidopsis or rice U6 promoters (Jiang, Zhou et al. 2013; Shan, Wang et al. 2013; Feng, Mao et al. 2014; Jiang, Yang et al. 2014). Ribozyme processing of transcripts from Pol II transcribed genes increases the flexibility of the system (Gao and Zhao 2014).

Plant genomes contain relatively large amounts of 5-methylcytosine (5meC; Kumar et al. 2013 J Genet 92(3): 629-666). Other than silencing transposable elements and repeated sequences, the biological roles of 5meC are still emerging. Intercrossing a low methylation mutant plant with a normally methylated plant resulted in heritable changes in DNA methylation in the plant genome that affected some plant phenotypic traits (Cortijo et al. 2014 Science. 2014 Mar 7;343(6175):1145-8). Over expression of Arabidopsis MET1, a DNA methyltransferase predominantly responsible for CG maintenance methylation, in Arabidopsis resulted in plants that flower earlier (U.S. Pat. Nos. 6,011,200 and 6,444,469). These methods are not gene specific in their methylation as methylation changes occur over a large part of the genome.

The ability to combine DNA modification enzymes with specific DNA binding proteins at specific DNA sequences creates new methods for targeted changes in DNA methylation, such as a TALEN-DNA demethylase in human cells (Maeder, Angstman et al. 2013). Protein fusions of sequence specific zinc finger or TALEN DNA binding proteins to Dnmt3a. or DNMT1 CG DNA methyltransferases have been used for targeted gene methylation in mammalian cells [(Li, Papworth et al, 2007; Siddique, Nunna. et al. 2013; Dyachenko, Tarlachkov et al. 2014; Nunna, Reinhardt et al. 2014) and references therein].

Circadian clock genes, CCA1, LHY, CHE, and TOC1, affect a plant's diurnal cycle and biochemistry, may play a role in heterosis in plants, and display some DNA methylation differences in parents and hybrid progeny (Ni, Kim et al. 2009; Ng, Miller et al. 2014). Alterations in CCA1 expression might be affected by DNA methylation levels (Ng, Miller et al. 2014) and have been proposed to affect heterosis (Ng, Miller et al. 2014), although the mechanisms of heterosis are not proven (Schnable and Springer 2013). Transgenic methods for CCA1 increased expression (U.S. Pat. No. 8,569,575) or decreased expression (US Pat Application No. 20140137290) are stated to increase plant yields.

Alterations in genomic DNA methylation can affect plant yields, but these examples are for genetically identical parents, as opposed to normal F1 heterosis between two genetically distinct parents (see U.S. Patent Application No. 20120284814, U.S. Provisional Application 61/863,267, U.S. Provisional Application 61/882,140, and U.S. Provisional Application 61/901,349, U.S. Provisional Application 61/930,602, U.S. Provisional Application 61/970424, U.S. Provisional Application 61/980096, and U.S. Provisional 61/983520, and U.S. Provisional 62/000756, each of which is incorporated by reference in its entirety, except that the claims and definitions sections are excluded from incorporation).

### Plant Transformation Methods.

Any of the recombinant DNA constructs provided herein can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation, Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton). Plant transformation methods for producing transgenic plants include, but are not limited to methods for: Alfalfa as described in U.S. Pat. No. 7,521,600; Canola and rapeseed as described in U.S. Pat. No. 5,750,871; Cotton as described in U.S. Pat. No. 5,846,797; corn as described in U.S. Pat. No. 7,682,829. Indica rice as described in U.S. Pat. No. 6,329,571; Japonica rice as described in U.S. Pat. No. 5,591,616; wheat as described in U.S. Pat. No. 8,212,109; barley as described in U.S. Pat. No. 6,100,447; potato as described in U.S. Pat. No. 7,250,554; sugar beet as described in U.S. Pat. No. 6,531,649; and, soybean as described in U.S. Pat. No. 8,592,212. Many additional methods or modified methods for plant transformation are known to those skilled in the art for many plant species

### SUMMARY OF INVENTION

In general, this invention generates useful DNA methylation increases in plants or plant cells and their progeny at one or more specific chromosomal regions. In certain embodiments plants or plant cells are subjected to expression of one or more targeted CG and/or CHG and/or CHH DNA methyltransferase fusion proteins, and said plants or their progeny are propagated via seeds or vegetatively, to produce plants with improved useful traits such as increased yield and/or tolerance to stress or disease. In general, the methods and compositions described herein provide useful and non-conventional methods to increase yields and useful traits in plants derived from progenitor plants or plant cells with increased DNA methylation at one or more specific chromosomal regions.

Methods for increasing cytosine methylation at targeted I)NA sequences in a plant or plant cell comprising the step of expressing a DNA methyltransferase fusion protein comprising a DNA methyltransferase domain and a DNA binding domain that binds one or more targeted DNA sequences in a plant or plant cell are provided herein.

Methods for producing and identifying a plant with increased cytosine methylation at targeted DNA sequences comprising the steps of: (a) expressing a DNA methyltransferase fusion protein comprising a DNA methyltransferase domain and a DNA binding domain that binds one or more targeted DNA sequences in a plant or plant cell; and, (b) selecting a plant or its progeny with increased DNA methylation at said targeted DNA sequences of step (a) are provided herein.

Methods of increasing cytosine methylation at targeted DNA sequences in a plant or plant cell comprising the step of expressing at least two types of DNA methyltransferase domains, wherein the types of DNA methyltransferase domains are selected from the DRM2, CMT2, CMT3, or MET1 types of DNA methyltransferases, and at least one of said DNA methyltransferase domains is fused to a DNA binding domain that binds one or more targeted DNA sequences.

In certain embodiments the DNA binding domain comprises the DNA binding domain of a member of the group consisting of a zinc finger, TALEN, or CRISPR protein. In certain embodiments the plant or plant cell comprises a sgRNA with homology to targeted DNA sequences and the DNA binding domain comprises a CRISPR/CAS9 protein. In certain embodiments the DNA methyltransferase domain comprises the catalytic methyltransferase domain of a member of the group consisting of CG, CHG, and/or CHH DNA methyltransferase protein. In certain embodiments the DNA methyltransferase domain comprises the catalytic methyltransferase domain of a member of the group consisting of a member of the MET1, DNMT3a, DNMT3b, DNMT1, DRM2, CMT2, or CMT1, or CMT3 family of proteins. In certain embodiments the DNA methyltransferase domain comprises the catalytic methyltransferase domain of a member of the group consisting of a member of the DRM2, CMT2, CMT1, CMT3, or MET1 family of proteins.

In certain embodiments of any of the aforementioned methods, the DNA methyltransferase catalytic domain is 95% to 100% homologous when aligned to the catalytic domain of a naturally occurring plant DRM2 protein, wherein an aligned amino acid position is considered identical if it contains an amino acid that is identical or a functionally conserved substitution or a conservatively modified variant of the amino acid being compared by alignment In certain embodiments of any of the aforementioned methods, the DNA methyltransferase catalytic domain is 95% to 100% homologous when aligned to the catalytic domain of a naturally occurring plant CMT2 protein, wherein an aligned amino acid position is considered identical if it contains an amino acid that is identical or a functionally conserved substitution or a conservatively modified variant of the amino acid being compared by alignment. In certain embodiments of any of the aforementioned methods, the DNA methyltransferase catalytic domain is 95% to 100% homologous when aligned to the catalytic domain of a naturally occurring plant CMT1 or CMT3 protein, wherein an aligned amino acid position is considered identical if it contains an amino acid that is identical or a functionally conserved substitution or a conservatively modified variant of the amino acid being compared by alignment. In certain embodiments of any of the aforementioned methods, the DNA methyltransferase catalytic domain is 95% to 100% homologous when aligned to the catalytic domain of a naturally occurring plant MET1 protein, wherein an aligned amino acid position is considered identical if it contains an amino acid that is identical or a functionally conserved substitution or a conservatively modified variant of the amino acid being compared by alignment.

In certain embodiments of any of the aforementioned methods, the progeny plant comprises heritable alterations in DNA methylation at targeted DNA sequences and does not contain a DNA methyltransferase fusion protein. In certain embodiments of any of the aforementioned methods, the targeted DNA sequence(s) comprise(s) one or more regions of a CCA1 and/or LHY gene(s). In certain embodiments, the CCA1 or LHY genes display increased DNA methylation at one or more promoter regions compared to a control CCA1 or LHY gene. In certain embodiments, the targeted DNA sequence s) comprise one or more regions of a CCA1 and/or LHY gene(s) and said CCA1 and/or LHY gene displays attenuated RNA transcript levels in a plant.

In certain embodiments of any of the aforementioned methods, the plant or plant cell comprises one or more DNA methyltransferase fusion proteins. In certain embodiments of any of the aforementioned methods, the plant or plant cell comprises one or more .DNA methyltransferase fusion proteins comprising a DNA binding domain of a CRISPR protein and a sgRNA with homology to one or more targeted DNA sequences. In certain embodiments of any of the aforementioned methods, the plant or plant cell comprises one or more DNA methyltransferase fusion proteins comprising a DNA binding domain of a CRISPR protein and a sgRNA with homology to one or more regions of a CCA1 and/or LHY gene(s). In certain embodiments of any of the aforementioned methods, the plant or plant cell comprises a DNA methyltransferase fusion protein comprises a catalytic methyltransferase domain of a member of the group consisting of a member of the DRM2, CMT2, CMT3, or MET1 family of proteins.

In certain embodiments of any of the aforementioned methods, the plant or plant cell comprises at least two types of DNA methyltransferase fusion proteins, wherein each type of DNA methyltransferase fusion protein comprises a DNA methyltransferase domain selected from the DRM2, CMT2, CMT1, CMT3, or MET1 types of DNA methyltransferases. In certain embodiments, the plant or plant cell comprises a targeted DNA binding domain that recruits a DNA methylation activity to one or more regions of CCA1 and/or LHY.

In certain embodiments of any of the aforementioned methods, expression is effected with a transgene comprising an inducible promoter that is operably linked to a DNA methyltransferase fusion protein coding region. In certain embodiments of any of the aforementioned methods, expression is effected with a transgene comprising a promoter that is operably linked to a DNA methyltransferase fusion protein coding region, wherein said promoter is a member of the group of promoters consisting of a MSH1, MET1, DRM2, CMT1, CMT2, or CMT3 plant promoter.

In certain embodiments, expression of a DNA methyltransferase fusion protein coding region is effected with an operably linked viral vector. In certain embodiments, expression of a DNA methyltransferase fusion protein is transiently expressed in a plant cell.

In certain embodiments of any of the aforementioned methods, a first and/or later generation progeny plant of step (b) exhibits one or more regions of pericentromeric CHG and/or CHH hypermethylation in comparison to a control plant not comprising or exposed to a DNA methyltransferase fusion protein. In certain embodiments of any of the aforementioned methods, the targeted DNA sequences have homology to one or more regions of pericentromeric regions or transposable elements in the plant host subjected to targeted DNA methylation.

In certain embodiments of any of the aforementioned methods, increased DNA methylation produces a useful trait selected from the group consisting of improved yield, delayed flowering, non-flowering, increased biotic stress resistance, increased abiotic stress resistance, enhanced lodging resistance, enhanced growth rate, enhanced biomass, enhanced tillering, enhanced branching, delayed flowering time, and delayed senescence in comparison to a control plant that had not been subjected to expression of a DNA methyltransferase fusion protein. In certain embodiments of any of the aforementioned methods, the selected plant(s) or progeny thereof exhibit an improvement in a trait in comparison to a plant that had not been subjected to expression of a DNA methyltransferase fusion protein but was otherwise isogenic to the first parental plant or plant cell.

In certain embodiments of any of the aforementioned methods, the plant is a crop plant. In certain embodiments of any of the aforementioned methods, the crop plant is selected from the group consisting of corn, soybean, cotton, wheat, rice, tomato, tobacco, millet, potato, sorghum, alfalfa, sunflower, canola, peanut, canola (Brassica napus, Brassica rapa ssp.), coffee (Coffea spp), coconut (Cocos nucijra), pineapple (Ananas comosus), citrus trees (Citrus spp.), cocoa (Theobroma cacao), poplar, sugar beets (Beta vulgaris), sugarcane Sacchanim spp.), oats, barley, vegetables, ornamentals, and conifers.

In certain embodiments of any of the aforementioned methods, the seed or a plant obtained therefrom exhibits an improvement in at least one useful trait. In certain embodiments of any of the aforementioned methods, the processed product from the plant or population of plants or from the seed thereof, comprises a detectable amount of a nuclear chromosomal DNA comprising one or more epigenetic changes that were induced by the DNA methyltransferase fusion protein. In certain embodiments of any of the aforementioned methods, the processed product is oil, meal, lint, bulls, or a pressed cake.

In certain embodiments of any of the aforementioned methods, plant exhibiting a useful trait is produced. In certain embodiments of any of the aforementioned methods, a clonal propagate derived from a plant or plant cell is produced. In certain embodiments of any of the aforementioned methods, a plant or progeny produced is grafted as a scion or rootstock. In certain embodiments, the progeny of a grafted plant produced by the aforementioned methods is produced.

In certain embodiments, plant or DNA construct comprising the DNA methyltransferase catalytic domain is 95% to 100% homologous when aligned to the catalytic domain of a naturally occurring plant DRM2, CMT1 CMT2, or CMT3 protein, wherein an aligned amino acid position is considered identical if it contains an amino acid that is identical or a functionally conserved substitution or a conservatively modified variant of the amino acid being compared by alignment is provided herein. In certain embodiments, plant or DNA construct comprising the DNA methyltransferase catalytic domain is 95% to 100% homologous when aligned to the catalytic domain of a naturally occurring plant MET1 protein, wherein an aligned amino acid position is considered identical if it contains an amino acid that is identical or a functionally conserved substitution or a conservatively modified variant of the amino acid being compared by alignment is provided herein.

In certain embodiments of any of the aforementioned methods, a plant and/or its progeny are provided. In certain embodiments of any of the aforementioned methods, the plant is from the group consisting of corn, wheat, rice, sorghum, millet, tomatoes, potatoes, soybeans, tobacco, cotton, alfalfa, rapeseed, sugar beets, sugarcane, sorghum, sunflower, peanut, canola (Brassica napus, Brassica rapa ssp,), coffee (Coffea spp.), coconut (Cocos nucijra), pineapple (Ananas comosus), citrus trees (Citrus spp.), cocoa (Theobroma cacao), poplar, sugar beets (Beta vulgaris), sugarcane (Saccharum spp), oats, barley, vegetables, ornamentals, and conifers.

### BRIEF DESCRIPTION OF THE DRAWINGS

FIG. 1A. Streptococcus (WP_002285322, NP_269215, Q99ZW2, WP_014736070 WP_001040076, G3ECR1.2, WP_002891502, WP_000428612, WP_002915084, and KEQ38765) proteins were aligned by clustal omega software. The sequence of a representative amino acid sequence (KEQ38765, which is SEQ ID NO:35) is shown for each genera, with the degree of conservation indicated by ‘.’ Or ‘:’ indicating conservative amino acid changes or ‘*’ indicating identical amino acids at this position.

FIG. 1B. Neisseria (WP_003684721.1, WP_002230835.1, WP_002260677.1, WP_009174359.1, WP_013449463.1, WP_003676410.1, WP_002238326.1, WP_002243824.1, WP_025460251.1, WP_019742773,1, WP_002246410.1, WP_002235162.1, and WP_002250828.1) proteins were aligned by clustal omega software. The sequence of a representative amino acid sequence (WP_002250828.1, which is SEQ ID NO:36) is shown for each genera, with the degree of conservation indicated by ‘.’ Or ‘:’ indicating conservative amino acid changes or indicating identical amino acids at this position.

FIG. 1C. Treponema (WP_002687349.1, WP_002684945.1, WP_010698457, WP_002692322.1, WP_002672887.1 WP_002676671.1, and WP_002681289.1) proteins were aligned by clustal omega software. The sequence of a representative amino acid sequence (WP_002681289.1, which is SEQ ID NO:37)_is shown for each genera, with the degree of conservation indicated by ‘.’ Or ‘:’ indicating conservative amino acid changes or ‘*’ indicating identical amino acids at this position.

FIG. 2. Alignment of representative Streptococcus, Neisseria, Treponema CRISPR/CAS9 proteins near the N-terminal RuvC-like and HNH-motif endonuclease catalytic regions wherein the locations of the D10A and H841A mutations are located to inactivate the nuclease domains of are marked in bold and underlined. (The protein domains and corresponding SEQ ID NO. are: Neisseria meningitides RuvC-like domain, SEQ ID NO:38; Streptococcus pyogenes RuvC-like domain, SEQ ID NO:39; Treponema denticola RuvC-like domain SEQ ID NO:40; Neisseria meningitides HNH-motif, SEQ ID NO:41; Streptococcus pyogenes HNH-motif, SEQ ID NO:42; Treponema denticola HNH-motif, SEQ ID No:43).

FIG. 3. Clustal Omega of the catalytic region of DNA methyltransferase protein sequences related to Arabidopsis MET1. The degree of amino acid conservation is indicated by ‘.’ Or ‘:’ indicating conservative amino acid changes or ‘*’ indicating identical amino acids at this position. The MET1 protein domains shown are of the following (species, genbank number, and corresponding SEQ ID NO.): Arabidopsis thaliana, NP_199727.1, SEQ ID NO:44, Arabidopsis lyrata, XP_002863965.1, SEQ ID NO:45; Capsella rubella, XP_006279892.1, SEQ ID NO:46; Brassica rapa, BAF34635.1, SEQ ID NO:47; Prunus persica, AAM96952.1, SEQ ID NO:48; Theobroma cacao, XP_007048602.1, SEQ ID NO:49, Medicago truncatula, XP_003619753.1, SEQ ID NO:50; Ricinus communis, XP_002518029.1, SEQ ID NO:51; Eucalyptus grandis, KCW54050.1, SEQ ID NO:52; Citrus sinensis, NP_001275841.1, SEQ ID NO:53; Solanum lycopersicum, NP_001234748.1, SEQ ID NO:54; Solanum tuberosurn, XP_006339355.1, SEQ ID NO:55, Aegilops tauschii, EMT23445.1, SEQ ID NO :56; Oryza saliva, EEE66687.1, SEQ ID NO:57; Zea mays, DAA59801.1, SEQ ID NO:58; Phaseolus vulgaris, XP_007152468.1 SEQ ID NO:59.

FIG. 4. Clustal Omega of the catalytic region of DNA methyltransferase protein sequences related to Arabidopsis CMT2. The degree of amino acid conservation is indicated by ‘.’ Or ‘:’ indicating conservative amino acid changes or ‘*’ indicating identical amino acids at this position. The CMT2 protein domains shown are of the following (species, genbank number, and corresponding SEQ ID NO.): Arabidopsis thaliana, NP_193637.2, SEQ ID NO:60; Capsella rubella, XP_006282433.1, SEQ ID NO:61; Eutrema salsugineum, XP_006414021.1, SEQ ID NO:62; Theobroma cacao, XP_007040779.1, SEQ ID NO:63; Prunus mume, XP_008238301.1, SEQ ID NO:64; Phaseolus vulgaris, XP 007156278.1, SEC) ID NO:65; Cucumis melo, XP_008448610.1, SEQ ID NO:66; Vitis vinifera, XP_002267685.2., SEQ ID NO:67; Glycine max, XP006599215.1_, SEQ ID NO:68; Fragaria vesca, XP 004301642.1, SEQ ID NO:69; Cicer arietinum, XP_004509555.1, SEQ ID NO:70; Medicago truncatula, KEH20304.1, SEQ ID NO:71; Populus x Canadensis, AHB20162.1, SEQ ID NO: 72; Eucalyptus grandis, KCW78468.1, SEQ ID NO:73; Solanum tuberosum, XP_006361281.1, SEQ ID NO:74; Ricinus communis, XP_002519960.1, SEQ ID NO:75; Oryza brachyantha, XP_006655109.1, SEQ ID NO:76; Gossypium hirsutum, AEC12443.1, SEQ ID NO:77; Oryza sativa, BAH37021.1, SEQ ID NO:78; Solanum lycopersicum, XP004228597.1, SEQ ID NO:79; Zea mays, NP_001104978, SEQ ID NO:80.

FIG. 5. Clustal Omega of the catalytic region of DNA methyltransferase protein sequences related to Arabidopsis CMT3. The degree of amino acid conservation is indicated by ‘.’ Or ‘:’ indicating conservative amino acid changes or ‘*’ indicating identical amino acids at this position. The CMT3 protein domains shown are of the following (species, genbank number, and corresponding SEQ ID NO.): Oryza sativa, EEE58631.1, SEQ ID NO:81; Hordeum vulgare, CAJ01708.1, SEQ ID NO:82; Sorghum bicolor, XP_002448525.1, SEQ ID NO:83; Zea mays, NP_001104978.1, SEQ ID NO:84; Arabidopsis thaliana, NP_177135.1, SEQ ID NO:85; Capsella rubella, XP_006300392.1, SEQ ID NO:86; Fragaria vesca, XP_004288717.1, SEQ ID NO:87; Ricinus communis, XP_002530367.1, SEQ ID NO:88; Solanum tuberosum, XP_006354167.1. SEQ ID NO:89; Solanum lycopersicum, XP 004252840.1, SEQ ID NO:90; Populus trichocarpa, XP_002299134.2, SEQ ID NO:91; Vitis vinifera, XP_002283355.2, SEQ ID NO:92; Citrus clementina, XP_006445885.1, SEQ ID NO:93; Citrus sinensis, NP_001275877.1, SEQ ID NO:94; Phaseolus vulgaris, XP_007152975.1, SEQ ID NO:95; Glycine max, XP_006572936.1 SEQ ID NO:96.

FIG. 6. Clustal Omega of the catalytic region of DNA methyltransferase protein sequences related to Arabidopsis DRM42. The degree of amino acid conservation is indicated by ‘.’ Or ‘:’ indicating conservative amino acid changes or ‘*’ indicating identical amino acids at this position. The DRM2 protein domains shown are of the following (species, genbank number, and corresponding SEQ ID NO.): Sorghum bicolor, XP 002468660.1, SEQ ID NO:97; Zea mays, NP 001104977, SEQ ID NO:98; Oryza sativa, ABF93591.1, SEQ ID NO:99; Aegilops tauschii, EMT00800.1, SEQ ID NO:100; Hordeum vulgare, BAJ96312.1, SEQ ID NO:101; Triticum urartu, EMS60441.1, SEQ ID NO:102; Arabidopsis thaliana, NP_196966.2, SEQ ID NO:103: Capsella rubella XP_006287272,1, SEQ ID NO:104; Fragaria vesca, XP_004304636.1, SEQ ID NO:105; Solanum tuberosurn, XP_006346949.1, SEQ ID NO:106; Solanum lycopersicum, XP_004237065.1, SEQ ID NO:107; Phaseolus vulgaris, XP_007151016.1, SEQ ID NO:108; Glycine max, XP_003524549.1, SEQ ID NO:109; Ricinus communis, XP_002521449,1, SEQ ID NO:110; Populus trichocarpa, XP_0023000462, SEQ ID NO: 111; Vitis vinifera, XP_002273972.2, SEQ ID NO:112; Citrus clementina, XP_006446539.1, SEQ ID NO:113; Citrus sinensis, AGU16983.1, SEQ ID NO:114.

FIG. 7 pCAMBIA1300-BAR.

FIG. 8. Plasmid Insert1 in pUC19.

FIG. 9. plasmid Insert2 in pUC19.

FIG. 10. plasmid Insert3 in binary pCAMBIA1300-BAR.

FIG. 11. plasmid Insert4 in pUC19.

FIG. 12. plasmid Insert5 in pUC19.

FIG. 13. plasmid Insert6 in binary pCAMBIA1300-BAR.

FIG. 14. plasmid Insert7 in binary pCAMBIA1300-BAR.

FIG. 15A. BLAST alignment of the soybean promoter regions of two CCA-like genes Glyma19g45030 (top strand, SEQ ID NO:115) and Glyma03g42260 (bottom strand, SEQ ID NO:116) upstream of the mRNA start sites to identify conserved regions suitable for targeting for sgRNAs for S. pyogenes CRISPR/CAS9. These sites are shown in bold and underlined and have the general format of A-N(18 or 19)-NGG, where A-N(18 or 19) is the target sequence for the sgRNA homology region.

FIG. 15B. BLAST alignment of the soybean promoter regions of two LHY-like genes Glyma16g01980 (top strand, SEQ ID NO:117) and Glyma07g05410 (bottom strand, SEQ ID NO:118) upstream of the mRNA start sites to identify conserved regions suitable for targeting for sgRNAs for S. pyogenes CRISPR/CAS9. These sites are shown in bold and underlined and have the general format of A-N(18 or 19)-NGG, where A-N(18 or 19) is the target sequence for the sgRNA homology region.

FIG. 16. plasmid Insert8 in pUC 19.

FIG. 17. plasmid Insert9 in binary pCAMBIA1300-BAR

FIG. 18. plasmid Insert10 in binary pCAMBIA1300-BAR (LHY-like).

FIG. 19. plasmid Insert11 in binary pCAMBIA1300-BAR (CCA1-like).

FIG. 20. plasmid Insert12 in binary pCAMBIA1300-BAR (CCA1-like).

FIG. 21. plasmid Insert13 in binary pCAMBIA1300-BAR (CCA1-like).

FIG. 22. plasmid Insert14 in binary pCAMBIA1300-BAR (CCA1-like),

FIG. 23. plasmid Insert15 in binary pCAMBIA1300-BAR (CCA1-like).

FIG. 24. plasmid Insert16 in binary pCAMBIA1300-BAR (CCA1-like).

FIG. 25. plasmid Insert17 in binary pCAMBIA1300-BAR (CCA1-like).

FIG. 26. plasmid Insert18 in binary pCAMBIA1300-BAR (CCA1-like).

FIG. 27. plasmid InsertGENERALIZED in binary pCAMBIA1300-BAR (LHY-like).

### DETAILED DESCRIPTION

### Definitions

As used herein, the phrases “CG altered gene” or “CG altered genes” refer to a gene or genes with increased levels of DNA methylation (5meC) at CG nucleotides within or near a gene or genes. The region near a gene is within 5,000 bp, preferably within 1,000 bp, of either the 5′ or 3′ end of the gene or genes.

As used herein, the phrases “clonal propagate” or “vegetatively propagated” refer to a plant or progeny thereof obtained from a plant, plant cell, tissue culture, or tissue, or seed that is propagated as a plant cutting or tuber cutting or tuber or tissue culture process such as embryogenesis or organogenesis. Clonal propagates can be Obtained by methods including but not limited to regenerating whole plants from plant cells, plant embryos, cuttings, tubers, and the like. Various techniques used for such clonal propagation include, but are not limited to, meristem culture, somatic embryogenesis, thin cell layer cultures, adventitious shoot culture, and callus culture.

As used herein, the phrases “commercially synthesized” or “commercial y available” DNA refer to the availability of any sequence of 15 bp up to 2000 bp in length or longer from DNA synthesis companies that provide a DNA sample containing the sequence submitted to them.

As used herein the phrase “Conservatively modified variants” includes individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Try (W); 7) Serine (S), Threonine (T); and 8) Cysteine (C), Methionine (M) (see, e.g., Creighton, Proteins (1984)).

As used herein, the phrase “crop plant” includes, but is not limited to, cereal, seed, grain, fruit, ornamental, and vegetable plants,

As used herein the phrase “DNA methyltransferase” refers to DNA methyltransferases of the broad DNMT1 evolutionary family (Xu et al., Curr Med Chem, 2010 ; 17(33):4052-4071; Law and Jacobsen, Nat Rev Genet. 2010 March ; 11(3): 204-220; Grace and Bestor Annu. Rev. Biochem. 2005,74:481-514), including DRM1 and DRM2, CMT1, CMT2, CMT3, and MET1.

As used herein, the phrase “developmental reprograming or the term “dr” refers to MSH1-dr like phenotypes.

As used herein, the phrase “DNA binding domain” refers to one or more protein domains of sequence-specific DNA binding proteins including, but not limited to, TALENS zinc fingers, and CRISPR/CAS9 proteins. For CRISPR/CAS9 proteins, the sequence-specific DNA binding proteins can be bound to sgRNAs to guide the sgRNA/protein complex to specific DNA binding sites.

As used herein, the phrase “DNA methyltransferase fusion protein” refers to a fusion protein comprising one or more proteins domains with DNA methyltransferase enzyme activity and one or more protein domains of specific DNA binding proteins including, but not limited to, TALENS, zinc fingers, and

As used herein the phrase “DNA methyltransferase fusion protein” refers to any fusion protein or gene encoding a protein that has DNA methyltransferase activity capable of methylating cytosine residues in DNA (C bases in DNA) at CHG and/or CHH sequences, and/or at CG positions. DNA methyltransferase fusion proteins include, but are not limited to, the DRM2 group, CMT2 group, CMT1 group, CMT3 group, and MET1 group of DNA methyltransferases and proteins or fusion proteins that contain catalytic domains of at least one of these DNA methyltransferases. In certain embodiments a DNA binding protein, including RNA-guided binding proteins such as CRISPR/CAS9 that bind DNA or KYP proteins that bind DNA, are fused to at either the N-terminus or C-terminus, with or without flexible peptide linkers such as GGGSS (SEQ ID NO:119) or GGSS (SEQ ID NO:120) or other flexible linkers used in protein fusions, of the catalytic domains of one or more of these DNA methyltransferases. For CRISPR/CAS9 proteins, specific DNA binding proteins can be bound to sgRNAs to guide the sgRNA/protein complex to specific DNA binding sites. DNA methyltransferase fusion proteins comprising a CRISPR/CAS9 protein domain function in protein/sgRNA complexes for binding to specific DNA sequences.

As used herein, the phrases “epigenetic modifications” or “epigenetic modification” refer to heritable and reversible epigenetic changes that include, but are not limited to, methylation of chromosomal DNA, and in particular, methylation of cytosine residues to 5-methylcytosine residues. Changes in DNA methylation of a region are often associated with changes in sRNA transcripts levels that are derived (have homology) to the methylated region.

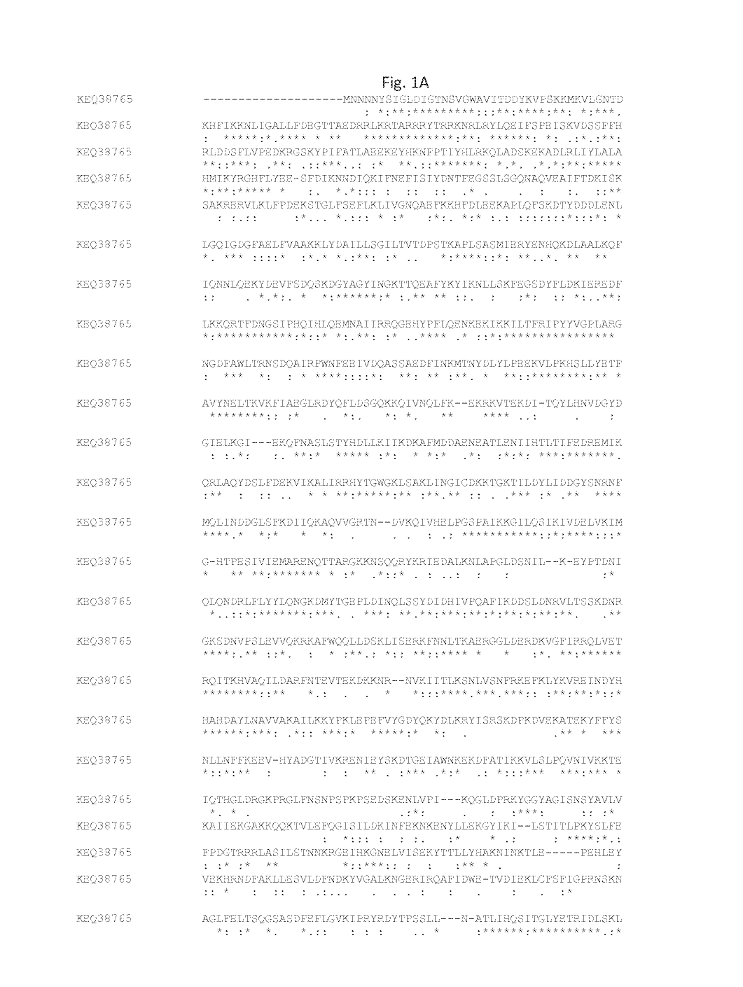

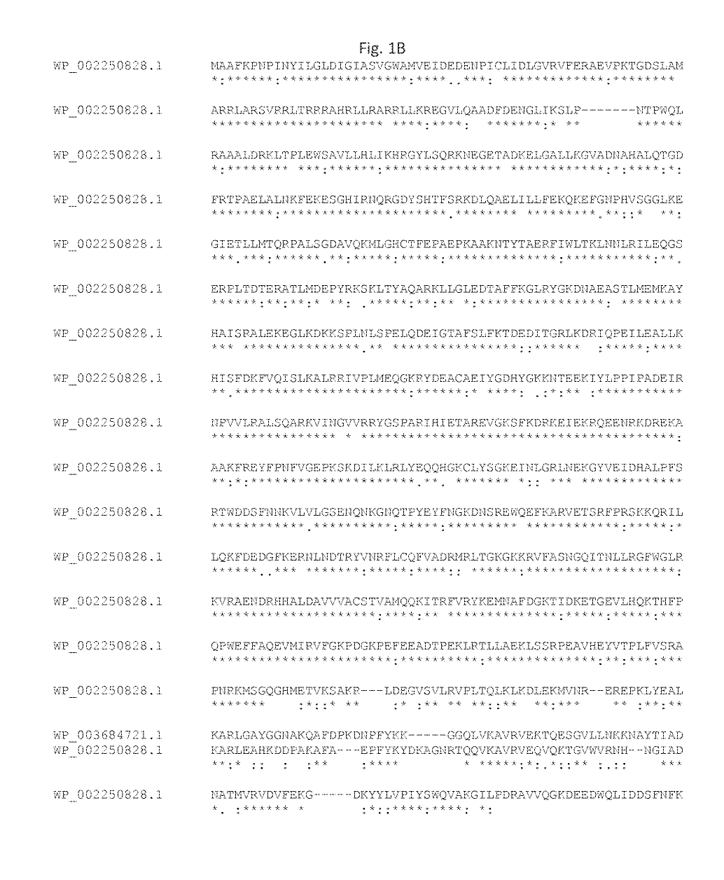

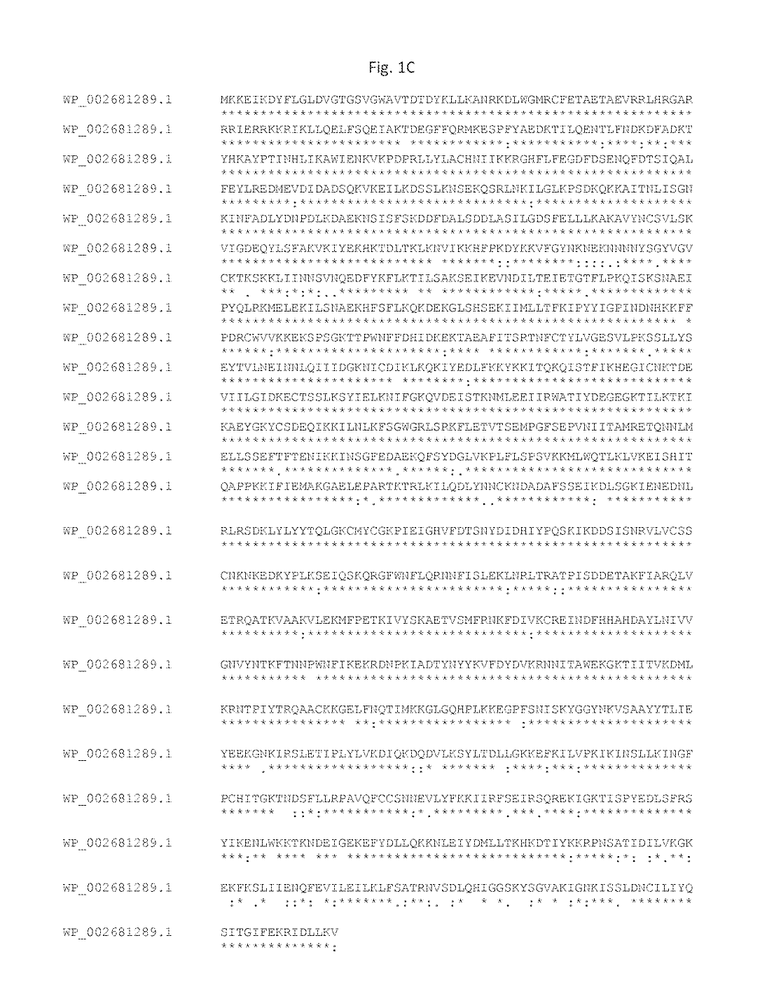

As used herein, the phrases “functionally conserved substitution” or“functionally conserved substitutions” refer to the amino acids that are present in clustal omega alignments of members of a protein family within a species or across multiple species. For example in FIG. 1 of DRM2 plant protein domains, in the most C-terminal sequence shown for AGU16983.1 (EGKESSLFYDYFRILDLVKNMMQRN-; SEQ ID NO:121) the following amino acids are observed to occur at the following positions and thereby are functionally conserved substitutions at these positions: E(E or G); G(G); K(K,D, or E); E(E,D,Q, or H); S(S); S(S or A); L(L); F(F); Y(Y, F, or H); D(D, E, H, or Q); Y(Y); F(F, C, V,or I); R(R): I(I or V); L(L or V); D(D, E, N, or H); L(L,V, I, A, H, or S); V(V); K(K or R); N(N, C, S, G,or A): M(M., I, L, R, A, E, A, or T); M(M, T, S, or Q); Q(Q, G, S, T, A, R, or E); R(R, K, N, T, G, A, or L); N(N, Y, H, R, Q, S, M, V, L or none end)). These evolutionarily allowed substitutions are functionally conserved substitutions, DRM1-related, DRM2-related, CMT1-related, CMT2-related, CMT3-related, MET1-related, or CRISPR/CAS-related proteins containing functionally conserved substitutions are generally functional even when their protein sequence is not identical.

As used herein, the term “F1” refers to the first progeny of two genetically or epigenetically different plants. “F2” refers to progeny from the self pollination of the F1 plant. “F3” refers to progeny from the self pollination of the F2 plant. “F4” refers to progeny from the self pollination of the F3 plant. “F5” refers to progeny from the self pollination of the F4 plant. “Fn” refers to progeny from the self pollination of the F(n-1) plant, where “n” is the number of generations starting from the initial F1 cross. Crossing to an isogenic line (backcrossing) or unrelated line (outcrossing) at any generation will also use the “Fn” notation, where “n” is the number of generations starting from the initial F1 cross.

As used herein, the phrases “genetically homogeneous” or “genetically homozygous” refer to the two parental genomes provided to a progeny plant as being essentially identical at the DNA sequence level.

As used herein, the phrases “genetically heterogeneous” or “genetically heterozygous” refers to the two parental genomes provided to a progeny plant as being substantially different at the sequence level. That is, one or more genes from the male and female gametes occur in different allelic forms with DNA sequence differences between them.

As used herein, the term “isogenic” refers to the two plants that have essentially identical genomes at the DNA sequence levels level.

As used herein, the phrase “heterotic group” refers to genetically related germplasm that produce superior hybrids when crossed to genetically distinct germplasm of another heterotic group.

As used herein, the phrase “heterologous sequence”, when used in the context of an operably linked promoter, refers to any sequence or any arrangement of a sequence that is distinct from the sequence or arrangement of the sequence with the promoter as it is found in nature. For example, an MSH1 promoter can be operably linked to a heterologous sequence that includes, but is not limited to, DNA methyltransferase fusion protein sequences.

“Homology” as used herein refers to sequence similarity between a reference sequence and at least a fragment of a second sequence. Homologs may be identified by any method known in the art, preferably, by using the BLAST or CLUSTAL Omega tool to compare a reference sequence or sequences to a single second sequence or fragment of a sequence or to a database of sequences. As described below, BLAST or CLUSTAL Omega will compare sequences based upon percent identity and similarity.

The terms “identical” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity or percent identity exists over a region that is at least about 50 nucleotides (or 10 amino acids) in length, or more preferably over a region that is 100 to 500 or 1000 or more nucleotides (or 20, 50, 200, or more amino acids) in length. Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. The BLASTN program (for nucleotide sequences or BLASTP program (for amino acid. sequences) or CLUSTAL Omega are suitable for most alignments.

As used herein, the phrases “increased DNA methylation” refers to nucleotides, regions, genes, chromosomes, and genomes located in the nucleus that have undergone an increase in 5meC (5-methyl cytosine) levels in a plant or progeny plant relative to the corresponding parental chromosomal loci prior to expression of a DNA methyltransferase fusion protein.

As used herein, the phrase “loss of function” refers to a diminished, partial, or complete loss of function.

As used herein, the phrases “MSH1-dr” or “MSH1-dr phenotypes” refers to one or more phenotypes that include leaf variegation, cytoplasmic male sterility (CMS), a reduced growth-rate phenotype, delayed or non-flowering phenotype, leaf wrinkling, increased plant tittering, decreased height, decreased internode elongation, plant tillering, and/or stomatal density changes that are observed in plants subjected to suppression of MSH1, but these phrases are applicable to plants with these phenotypes regardless of how the plants were produced.

As used herein, the phrase “new combinations of DNA methylation regions” refers to nuclear chromosomal regions in a progeny plant with one or more differences in :DNA methylation levels when compared to chromosomal loci of a parental plant if derived by self-pollination, or if derived from a cross, when compared to either parental plant, each compared separately to said progeny plant.

As used herein, the term “non-regenerable” refers to a plant part or plant cell that cannot give rise to a whole plant.

The phrase “operably linked” as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. If the linkage of the promoter to the coding sequence is a transcriptional fusion and expression of the encoded protein is desired, the linkage is made so that the first translational initiation codon in the resulting transcript is the initiation codon of the coding sequence. Alternatively, if the linkage of the promoter to the coding sequence is a translational fusion and expression of the encoded protein is desired, the linkage is made so that the first translational initiation codon contained in the 5′ untranslated sequence associated with the promoter is linked such that the resulting translation product is in frame with the translational open reading frame that encodes the protein desired. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators, sequences that provide DNA transfer and/or integration functions (i.e., site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences, homologous recombination sequences), and sequences that provide replication functions (i.e., bacterial origins of replication, autonomous replication sequences, centromeric sequences).

As used herein, the terms “pericentromeric” or “pericentromere” refer to heterochromatic regions containing abundant repeated sequences, transposable elements, and retrotransposons that physically flank the centromeric regions. At the sequence level, a functional definition for pericentromeric sequences are highly repeated sequences that contain transposable elements and retrotransposons embedded in said repeated sequences. When known, centromeric repeats can be computationally removed from the repeated sequences, but their presence is not detrimental if not computationally removed. When available, chromosomal positioning information about the location of sequences that are located adjacent to the centromere can be used as an additional criteria for pericentromeric sequences.

As used herein, the terms “polynucleotide,” “nucleic acid”, “nucleic acid sequence,” “sequence of nucleic acids,” and variations thereof shall be generic to polydeoxyribonucleotides (containing 2-deoxy-D-ribose), to polyribonucleotides (containing D-ribose), to any other type of potynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators metals, radioactive metals, boron, oxidative metals, etc.). As used herein, the symbols for nucleotides and polynucleotides are those recommended by the IUPAC-IUB Commission of Biochemical Nomenclature (Biochem: 9:4022, 1970).

As used herein, the term “progeny” refers to any one of a first, second, third, or subsequent generation obtained from a parent plant if self-pollinated or from parent plants if obtained from a cross, or through any combination of selfing and crossing. Any materials of the plant, including but not limited to seeds, tissues, pollen, and cells can be used as sources of RNA or DNA for determining the status of the RNA or DNA composition of said progeny.

As used herein_{;} the phrase “reference plant” refers to a parental plant or progenitor of a parental plant prior to expression of a DNA methyltransferase fusion protein, but otherwise isogenic to the candidate or test plant to which it is being compared. In across of two parental plants, a “reference plant” can also be from parental plants wherein expression of a DNA methyltransferase fusion protein was not used in said parental plants or their progenitors.

As used herein, the term “S1” refers to a first selfed plant. “S2” refers to progeny from the self pollination of the S1 plant, “S3” refers to progeny from the self pollination of the S2 plant. “S4” refers to progeny from the self pollination of the S3 plant. “S5” refers to progeny from the self pollination of the S4 plant. “Sn” refers to progeny from the self pollination of the S(n-1) plant, where “n” is the number of generations starting from the initial S1 cross.

As used herein, the terms “self”, “selfing”, or “selfed” refer to the process of self pollinating a plant.

As used herein, the term “transgene” or “transgenic” refers to any recombinant DNA that has been transiently introduced into a cell or stably integrated into a chromosome or minichromosome that is stably or semi-stably maintained in a host cell. In this context, sources for the recombinant DNA in the transgene include, but are not limited to, DNAs from an organism distinct from the host cell organism, species distinct from the host cell species, varieties of the same species that are either distinct varieties or identical varieties, DNA that has been subjected to any in vitro modification, in vitro synthesis, recombinant DNA, and any combination thereof. The terms transgene or transgenic include inserting or changing DNA sequences at endogenous genes to alter their expression or function through any non-natural process.

As used herein, the phrases “useful for plant breeding” or “useful for breeding” refer to plants derived from one or more progenitor plants or plant cells that were subjected to expression of a DNA methyltransferase fusion protein that are useful in a plant breeding program for the objecting of developing improved plants and plant seeds to a greater extent than control plants not subjected to expression of a DNA methyltransferase fusion protein or derived from progenitor plants subjected to expression of a DNA methyltransferase fusion protein.

As used herein, the phrases “useful trait” or “useful traits” refer to plants derived from one or more progenitor plants that were subjected to expression of a DNA methyltransferase fusion protein that exhibit one or more agriculturally useful traits to a greater extent than control plants not subjected to expression of a DNA methyltransferase fusion protein or derived from progenitor plants subjected to expression of a DNA methyltransferase fusion protein.

As used herein, the phrases “targeted DNA sequence” or “targeted DNA sequences” refer to one or more DNA sequence to which a DNA methyltransferase fusion protein is intended to bind.

As used herein, the phrase “targeted DNA methylation refers to a method of using a DNA methyltransferase fusion protein or other fusion protein capable of specifically binding DNA and recruiting DNA methyltransferase activity to cause increased DNA methylation at the targeted DNA sequence(s).

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Identification of DRM2 group, CMT2 group, CMT1, CMT3 group, or MET1 group DNA methyltransferases

Orthologous DRM1, DRM2, CMT2, CMT1, CMT3, or MET1, or other DNA methyltransferase genes related to these proteins can be obtained from many crop species through the BLAST comparison of the protein sequences known members of these proteins to the genomic databases (NCBI and publically available genomic databases for specific crop species). Specifically the genome, cDNA, or EST sequences are available for apples beans, badey, Brassica napus, rice, Cassava, Coffee, Eggplant, Orange, sorghum, tomato, cotton, grape, lettuce, tobacco, papaya, pine, rye, soybean, sunflower, peach, poplar, scarlet bean, spruce, cocoa, cowpea, maize, onion, pepper, potato, radish, sugarcane, wheat, and other species at the following internet or world wide web addresses : “compbio.dfci.harvard.edu/tgi/plant.html”; “genomevolution.org/wiki/index.php/Sequenced_plant_genomes”; “ncbi.nlm.nih.gov/genomes/PLANTS/PlantList.html”; “plantgdb.org/”; “arabidopsis.org/portals/genAnnotation/other_genomes/”; “gramene.org/resources/”; “genomenewsnetwork.org/resources/sequenced_genomes/genome_guide_p1.shtml”; “jgi.doe.gov/programs/plants/index.jsf”; “chibba.agtec.uga.edu/duplication/”; “mips.helmholtz-muenchen.de/plant/genomes.jsp”; “science.co.il/biomedical/Plant-Genome-Databases.asp”; “jcvi.org/cms/index.php?id=16”; and “phyto5.phytozome.net/Phytozome_resources.php”.

Plant and non-plant CG, CHG, or CHH DNA methyltransferases are suitable for use in the present invention. Candidate genes or proteins can be aligned by BLAST or Clustal Omega. Candidate genes encoding proteins with 50%-70%, 70%-80%, 80%-90%, 90%-95%, or 95% -100% identity to known members of these proteins and that have DNA methyltransferase activity are considered useful DNA methyltransferases for the present invention. Conservatively modified variants of these DNA methyltra.nsferases occur naturally or can be intentionally modified by recombinant DNA methods and still be contemplated by the present invention.

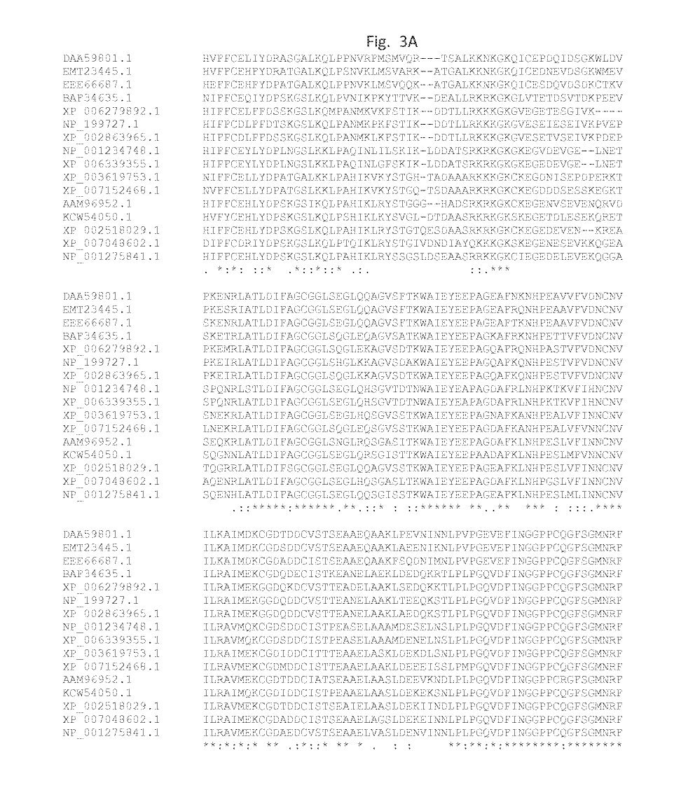

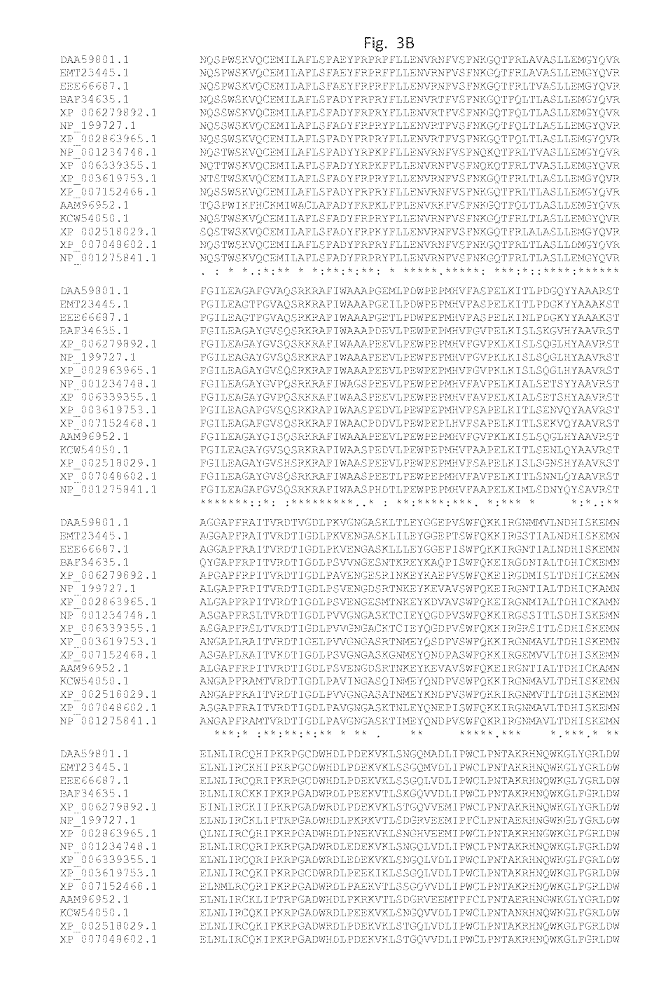

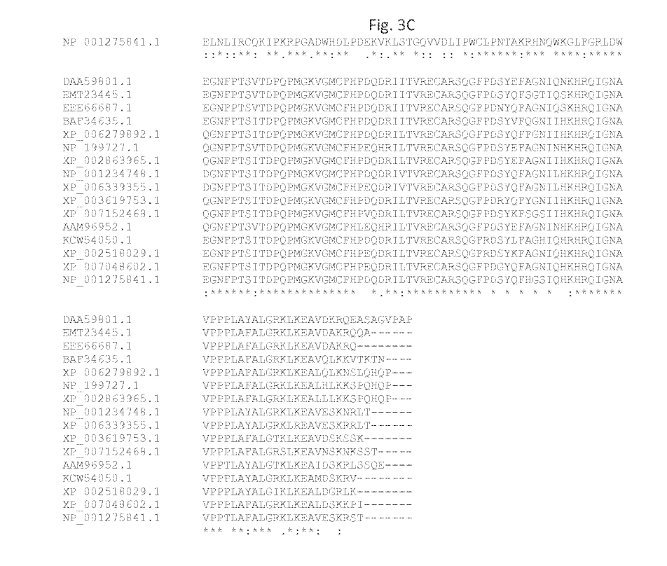

In certain embodiments, the DNA methyltransferase fusion protein of the invention, comprising a DNA. binding domain for DNA sequence specific targeting and a DNA methyltransferase domain, for which said DNA methyltransferase domain has at least about 90%-95%, or 95% -100% amino acid residue sequence identity to the catalytic regions of one of the proteins in FIGS. 3-6 or a protein related to these that contains identical or functionally conserved substitutions or conservatively modified variants at each equivalent amino acid position in the conserved catalytic region. In preferred embodiments, the polynucleotides of the invention encode polypeptides having at least about 90%-95%, or 95% -100% amino acid. residue sequence identity to the catalytic regions of one of the proteins in FIGS. 3-6 or a protein related to these that contains identical or functionally conserved substitutions or conservatively modified variants at each equivalent amino acid position in the conserved catalytic region. certain embodiments polynucleotides of the invention further include polynucleotides that encode conservatively modified ⁻variants of potypeptides encoded by proteins listed in FIGS. 3-6, and homologous or orthologous genes or proteins of other plant species. In certain embodiments, the recombinant polynucleotides of the invention encode proteins that have 90%-95%, or 95% -100% amino acid residue sequence identity to identical or functionally conserved substitutions or conservatively modified variant amino acids of DNA methyltransferase polypeptides at the amino acids positions of the catalytic regions in FIGS. 3-6.

Methods for obtaining DNA methyltransferase genes include, but are not limited to, techniques such as: i) searching amino acid and/or nucleotide sequence databases to identify the DNA methyltransferases genes by sequence identity comparisons; ii) cloning the DNA methyltransferases gene by either PCR from genomic sequences or RT-PCR from expressed RNA; iii) cloning the DNA methyltransferases target gene from a genotnic or cDNA library using PCR and/or hybridization based techniques; iv) cloning the DNA methyltransferases target gene from an expression libraty where an antibody directed ⁻to the DNA methyltransferases target gene protein is used to identify the DNA methyltransferases target gene containing clone; v) cloning the DNA methyltransferases target gene by complementation of an DNA methyltransferases target gene mutant or DNA methyltransferases gene deficient plant; or vi) any combination of (i), (ii), (iv), and/or (v). The DNA sequences of the target genes can be obtained from the promoter regions or transcribed regions of the target genes by PCR isolation from genomic DNA, or PCR of the cDNA for the transcribed regions, or by commercial synthesis of the DNA sequence. RNA sequences can be chemically synthesized or, more preferably, by transcription of suitable DNA templates. Confirming that the candidate DNA methyltransferases target gene can methylate DNA in plants can he readily determined or confirmed by constructing a plant transformation vector that provides for expression of the target gene, transforming the plants with the vector, and determining if plants transformed with the vector exhibit increased DNA methylation. Additionally, diagnostic phenotypes include those that are typically observed in various plant species when epigenetic marks are perturbed, including leaf variegation, cytoplasmic male sterility (CMS), a reduced growth-rate phenotype, delayed or non-flowering phenotype, and enhanced susceptibility to pathogens. These characteristic responses have been described previously as developmental reprogramming or “MSH1-dr” (Xu et al. Plant Physiol. Vol. 159:711-720, 2012).

In general, methods provided herewith for introducing epigenetic variation in plants require plants or plant cells to be subjected to expression of a DNA methyltransferase fusion protein for a time sufficient in the entire plant or in appropriate subsets of cells (i.e meristematic and/or floral cells). As such, a wide variety of methods of expressing a DNA methyltransferase fusion protein can be employed to practice the methods provided herewith and the methods are not limited to a particular expression technique.

In certain embodiments, DNA methyltransferase fusion protein genes may be used directly in either a homologous or a heterologous plant species to provide for expression of a DNA methyltransferase fusion protein gene in either the homologous or heterologous plant species. A transgene from Arabidopsis or rice or soybean or other plant species that provides for expression of a DNA methyltransferase fusion protein can be used in certain embodiments in millet, sorghum, and maize, or other plants including, but not limited to, cotton, canola, wheat, barley, flax, oat, rye, turf grass, sugarcane, alfalfa, banana, broccoli, cabbage, carrot, cassava, cauliflower, celery, citrus, a cucurbit, eucalyptus, garlic, grape, onion, lettuce, pea, peanut, pepper, potato, poplar, pine, sunflower, safflower, soybean, strawberry, sugar beet, sweet potato, tobacco, cassava, cauliflower, celery, citrus, cotton, a cucurbit, eucalyptus, garlic, grape, onion, lettuce, pea, peanut, pepper, potato, poplar, pine, sunflower, safflower, strawberry, sugar beet, sweet potato, tobacco, cassava, cauliflower, celery, citrus, cucurbits, eucalyptus, garlic, grape, onion, lettuce, pea, peanut, pepper, poplar, pine, sunflower, safflower, soybean, strawberry, sugar beet, tobacco, Jatropha, Camelina, and Agave.

Inducible DNA. methyltransferase fusion protein expression can be with promoters that include, but are not limited to, a PR-1a promoter (US Patent Application Publication Number 20020062502) or a GST II promoter (WO 1990/008826 A1). Additional examples of inducible promoters include, without limitation, the AdhI promoter which is inducible by hypoxia or cold stress, the Hsp70 promoter which is inducible by heat stress, and the PPDK promoter which is inducible by light. In other embodiments, a transcription factor that can be induced or repressed as well as a promoter recognized by that transcription factor and operably linked to the DNA methyltransferase fusion protein sequences are provided. Such transcription factor/promoter systems include, but are not limited to: i) DNA binding-activation domain-ecdysone receptor transcription factors/cognate promoters that can be induced by methoxyfenozide, tebufenozide, and other compounds (US Patent Application Publication Number 20070298499); ii) chimeric tetracycline repressor transcription factors/cognate chimeric promoters that can be repressed or de-repressed with tetracycline (Gatz, C., et al. (1992). Plant J. 2, 397-404), estradiol or dexamethasone inducible promoters (Aoyama and Chua, The Plant Journal (1997) 11(3):605-612; Zuo et al., The Plant Journal (2000) 24(2):265-273), and the like.

In certain embodiments, a promoter that provides for selective expression of a DNA methyltransferase fusion protein in specific cells is used. In certain embodiments, this promoter is an Msh1 or a PPD3 promoter. In certain embodiments, this promoter is a meristem active promoter such as CAMV 35S promoter, the FMV 34/35 S promoter, the rice Actin promoter, the maize ubiquitin promoter, or floral active promoters and an operably linked DNA methyltransferase fusion protein coding region. Such promoters that can be used to express DNA methyltransferase fusion proteins include, but are not limited to, Arabidopsis, sorghum, tomato, rice, and maize promoters as well as functional derivatives thereof that likewise provide for expression in meristematic or reproductive cells. In certain embodiments, recombinant DNA constructs for expression of DNA methyltransferase fusion protein can comprise a promoter from a dicotyledonous species such as Arabidopsis, soybeans or canola, or monocotyledonous species such as rice, maize or sorghum operably attached to a DNA methyltransferase fusion protein coding region followed by a polyadenylation region. Various 3′ polyadenylation regions known to function in monocots and dicot plants include, but are not limited to, the Nopaline Synthase (NOS) 3′ region, the Octapine Synthase (OCS) 3′ region, the Cauliflower Mosaic Virus 35S 3′ region, the Mannopine Synthase (MAS) 3′ region. In certain embodiments recombinant DNA constructs for expression of monocot target genes can comprise a promoter from a monocot species such as rice, maize, sorghum or wheat attached to a monocot intron before the DNA methyltransferase fusion protein coding region. Monocot introns that are beneficial to gene expression when located between the promoter and coding region are the first intron of the maize ubiquitin (described in U.S. Pat. No. 6,054,574) and the first intron of rice actin 1 (McElroy, Zhang et al. 1990). Additional introns that are beneficial to gene expression when located between the promoter and coding region are the maize hsp70 intron (described in U.S. Pat. No 5,859,347), and the maize alcohol dehydrogenase 1 genes introns 2 and 6 (described in U.S. Pat. No. 6,342,660).

In still other embodiments, transgenic plants are provided wherein the transgene that provides for DNA methyltransferase fusion protein expression is flanked by sequences that provide for removal for the transgene. Such sequences include, hut are not limited to, transposable element or recombinase sequences that are acted on by a cognate transposase or recombinase. Non-limiting examples of such recombinase systems that have been used in transgenic plants include the cre-lox and FLP-FRT systems.

DNA methyltransferase fusion protein gene expression can be readily identified or monitored by molecular techniques. Molecular methods for monitoring DNA methyltransferase fusion protein target gene RNA expression levels include, but are not limited to, use of semi-quantitive or quantitative reverse transcriptase polymerase chain reaction (qRT-PCR) techniques. Various quantitative RT-PCR procedures including, but not limited to, TaqMan.™. reactions (Applied Biosystems, Foster City, Calif. US), use of Scorpion.™. or Molecular Beacon.™. probes, or any of the methods disclosed in Bustin, S. A. (Journal of Molecular Endocrinology (2002) 29, 23-39) can be used. It is also possible to use other RNA quantitation techniques such as Quantitative Nucleic Acid Sequence Based Amplification (Q-NASBA.™.) or the Invader.™. technology (Third Wave Technologies, Madison, Wis.).

Alterations of endogenous plant DNA methyltransferase target genes to produce DNA methyltransferase fusion protein genes can be obtained from a variety of sources and by a variety of techniques. A homologous replacement sequence containing one or more alterations and homologous sequences at both ends of the double stranded break can provide for homologous recombination and substitution of the resident wild-type DNA methyltransferase target gene sequence in the chromosome with a replacement sequence fusion to a DNA binding domain. Gain of function alterations include, but are not limited to, overexpression of the target gene or fragments thereof and/or fusions of DNA binding proteins, including CRISPR-CAS9 types, to the endogenous DNA methyltransferase fusion proteins.

Methods for substituting endogenous chromosomal sequences by homologous double stranded break repair have been reported in tobacco and maize (Wright et al., Plant J. 44, 693, 2005; D'Halluin, et al., Plant Biotech. J. 6:93, 2008). A homologous replacement can also be introduced into a targeted nuclease cleavage site by non-homologous end joining or a combination of non-homologous end joining and homologous recombination (reviewed in Puchta, J. Exp. Bot. 56; 1, 2005; Wright et al., Plant J. 44; 693, 2005). In certain embodiments, at least one site specific double stranded break can be introduced into the endogenous DNA methyltransferase gene by a meganuclease. Genetic modification of meganucleases can provide for meganucleases that cut within a recognition sequence that exactly matches or is closely related to specific endogenous DNA methyltransferase gene sequence (WO/06097853A1, WO/06097784A1, WO/04067736A2, U.S. 20070117128A1). It is thus anticipated that one can select or design a nuclease that will cut within a target DNA methyltransferase target gene sequence. In other embodiments, at least one site specific double stranded break can be introduced in the endogenous DNA methyltransferase target gene target sequence with a zinc finger nuclease. The use of engineered zinc finger nuclease to provide homologous recombination in plants has also been disclosed (WO 03/080809, WO 05/014791, WO 07014275, WO 08/021207). In still other embodiments, CRISPR/CAS9 systems are used for genome editing to create mutations or gene replacement and modifications alterations (Strauβ and Lahaye, Mol Plant. 2013 Sep:6(5):1384-7; Sampson and Weiss Bioessays 2014 Jan;36(1):34-8).

Any of the recombinant DNA constructs provided herein can be introduced into a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Aforementioned methods of introducing transgenes are well known to those skilled in the art and are described in U.S. Patent Application No, 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety. Methods of using bacteria such as Rhizobium or Sinorhizobium to transform plants are described in Broothaerts, et al., Nature. 2005,10;433(7026):629-33. It is further understood that the recombinant DNA constructs can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Expression from transiently expressed genes or mRNAs or expression from viral genomes can also be used. Those skilled in the art will further appreciate that any of these gene transfer techniques can be used to stably or transiently introduce the recombinant DNA. constructs into the nucleus or chromosome of a plant cell, a plant tissue or a plant.

Methods of introducing plant minichromosomes comprising plant centromeres that provide for the maintenance of the recombinant minichromosome in a transgenic plant can also be used in practicing this invention (U.S. Pat. No. 6,972,197 and US Patent Application Publication 20120047609). In these embodiments of the invention, the transgenic plants harbor the minichromosotnes as extrachromosomal elements that are not integrated into the chromosomes of the host plant. It is anticipated that such mini-chromosomes may be useful in providing for variable transmission of a resident recombinant DNA construct that expresses a DNA methyltransferase fusion protein.

Methods where DNA methyltransferase fusion protein expression or genome edited expression or alteration is effected in cultured plant cells are also provided herein. In certain embodiments, DNA methyltransferase fusion protein expression or genome edited expression or alteration is effected in cultured plant cells by introducing a nucleic acid that provides for such expression in the plant cells. Nucleic acids that can be used to provide for expression in cultured plant cells include, but are not limited to, transgenes, mRNA, and recombinant virus vectors.

Nucleic acid or protein molecules that provide DNA methyltransferase activity can be introduced by electroporation or particle gun or other physical methods or Agrobacterium or Rhizobium gene transfer methods. The expression of the plant DNA methyltransferase fusion protein genes in cultured plant cells is specifically provided herein,

DNA methyltransferase fusion protein expression can also be readily identified or monitored by traditional methods where plant phenotypes are observed. For example, DNA methyltransferase fusion protein gene function can be identified or monitored by observing epigenetic effects that include leaf variegation, cytoplasmic male sterility (CMS), a reduced growth-rate phenotype, delayed or non-flowering phenotype, and/or enhanced susceptibility to pathogens. Phenotypes indicative of epigenetic phenotypes in various plants are provided in WO 2012/151254, which is incorporated herein by reference in its entirety, Epigenetic variation can also produce changes in plant tillering, height, internode elongation and stomatal density (referred to herein as “MSH1-dr” phenotypes) that can be used to identify or monitor epigenetic effects in plants. Other biochemical and molecular traits can also be used to identify or monitor epigenetic effects in plants. Such molecular traits can include, but are not limited to, changes in expression of genes involved in cell cycle regulation, Giberrellic acid catabolism, auxin biosynthesis, auxin receptor expression, flower and vernalization regulators (i.e. increased FLC and decreased SOC1 expression), as well as increased miR156 and decreased miR172 levels. Such biochemical traits can include, but are not limited to, up-regulation of most compounds of the TCA, NAT) and carbohydrate metabolic pathways, down-regulation of amino acid biosynthesis, depletion of sucrose in certain plants, increases in sugars or sugar alcohols in certain plants, as well as increases in ascorbate, alphatocopherols, and stress-responsive flavones apigenin, and apigenin-7-oglucoside, isovitexin, kaempferol 3-O-beta-glucosi de, luteolin-7-O-glucoside, and vitexin. It is further contemplated that in certain embodiments, a combination of both molecular, biochemical, and traditional methods can be used to identify or monitor epigenetic effects in plants. It is further contemplated that in certain embodiments, plants displaying one or more Msh1-dr phenotypes in at least a portion of said plants can be outcrossed or selfed to obtain progeny plants lacking DNA methyltransferase fusion protein genes or proteins and exhibiting enhanced growth or yields or useful traits in the F1, F2, F3, or Fn generations.

Expression of one or more DNA methyltransferase fusion proteins that results in useful epigenetic changes and useful traits can also be readily identified or monitored by assaying for characteristic DNA methylation and/or gene transcription and/or sRNA patterns that occur in plants subject to such perturbations. In certain embodiments, characteristic DNA methylation and/or gene transcription and/or sRNA patterns that occur in plants subject to expression of a DNA methyltransferase fusion protein can be monitored in a plant, a plant cell, plants, seeds, and/or processed products obtained therefrom to identify or monitor effects mediated by expression of a DNA methyltransferase fusion protein. Expression of DNA methyltransferase fusion protein results in: hypermethylation of CG, CHG, and CHH chromosomal positions and regions. In certain embodiments, expression of DNA methyltransferase fusion protein in the plant species being analyzed for DNA methylation changes provides altered chromosomal loci with altered DNA methylation patterns. In certain n embodiments, first or second or later generation progeny of a plant subjected to expression of a DNA methyltransferase fusion protein will exhibit CG differentially methylated regions (DMR) of various discrete targeted chromosomal loci that include, but are not limited to, the MSH1 locus and changes in plant defense and stress response gene expression. In certain embodiments, a plant, a plant cell, a seed, plant populations, seed populations, and/or processed products obtained therefrom that has been subject to expression of a DNA methyltransferase fusion protein will exhibit pericentromeric or repeated sequence or transposable element CHG and/or CHH hypermethylation and/or CG hypermethlation of various targeted chromosomal regions.

Such CHG and/or CHH hypermethylation is understood to be methylation at the sequence “CHG” or “CHH” where H=A, T, or C. Such CG and CHG and CHH hypermethylation can be assessed by comparing the methylation status of a sample from plants or seed that had been subjected to expression of a DNA methyltransferase fusion protein, or a sample from progeny plants or seed derived therefrom, to a sample from control plants or seed that had not been subjected to expression of a DNA methyltransferase fusion protein. It is further contemplated that in certain embodiments, plants subjected to expression of a DNA methyltransferase fusion protein displaying altered chromosomal loci in at least a portion of said plants can be outcrossed or selfed to obtain progeny plants lacking a DNA methyltransferase fusion protein gene and exhibiting enhanced growth or yields or useful traits in the F1, F2, F3, or Fn generations.

A variety of methods that provide for functional expression of a DNA methyltransferase fusion protein in a plant followed by recovery of progeny plants not expressing a DNA methyltransferase fusion protein and with useful epigenetic changes are provided herein. In certain embodiments, progeny plants can be recovered by downregulating expression of a DNA methyltransferase fusion protein or by removing the DNA methyltransferase fusion protein transgene with a transposase or recombinase. In certain embodiments of the methods provided herein, a DNA methyltransferase fusion protein gene is functionally suppressed or removed from a target plant or plant cell and progeny plants by genetic techniques. In one exemplary and non-limiting embodiment, progeny plants can be obtained by selfing a plant that is heterozygous for the transgene that provides for expression of a DNA methyltransferase fusion protein by segregation. Selfing of such heterozygous plants o. selfing of heterozygous plants regenerated from plant cells) provides for the transgene to segregate out of a subset of the progeny plant population. Where a DNA methyltransferase fusion protein gene is derived by a dominant mutation in an endogenous gene the plant can, in yet another exemplary and non-limiting embodiment, be selfed if heterozygous or crossed to wild-type plants if homozygous and then selfed to obtain progeny plants that are homozygous for a functional, wild-type DNA methyltransferase gene allele. In other embodiments, plant cell and/or progeny plants that lack expression of or lack the DNA methyltransferase fusion protein gene are recovered by molecular genetic techniques. Non limiting and exemplary embodiments of such molecular genetic techniques include: i) downregulation of expression under the control of a regulated promoter by withdrawal of an inducer required for activity of that promoter or introduction and/or induction of a repressor of that promoter; or, ii) exposure of the transgene flanked by transposase or recombinase recognition sites to the cognate transposase or recombinase that provides for removal of that transgene.

In certain embodiments of the methods provided herein, progeny plants derived from plants subjected to functional expression of a DNA methyltransferase fusion protein exhibit male sterility, dwarfing, variegation, and/or delayed flowering time and lack a DNA methyltransferase fusion protein gene are obtained and maintained as independent breeding lines or as populations of plants. Certain individual progeny plant lines obtained from the outcrosses of plants where expression of a DNA methyltransferase fusion protein occurred to other plants can exhibit useful phenotypic variation where one or more traits are improved relative to either parental line and can be selected. Useful phenotypic variation that can be selected in such individual progeny lines includes, but is not limited to, increases in fresh and dry weight biomass and/or seed or fruit yield relative to either parental line.

Individual lines obtained from plants wherein expression of a DNA methyltransferase fusion protein occurred can also be selfed to obtain progeny plants that lack the phenotypes that can be associated with epigenetics (i.e. male sterility, dwarfing, variegation, and/or delayed flowering time). Recovery of such progeny plants that lack the undesirable phenotypes can in certain embodiments be facilitated by removal of the transgene or endogenous locus that provides for expression of a DNA methyltransferase fusion protein. In certain embodiments, progeny of such selfs can be used to obtain individual progeny lines or populations that exhibit significant useful phenotypic variation. Certain individual progeny plant lines or populations Obtained from selfing plants where expression of a DNA methyltransferase fusion protein occurred can exhibit useful phenotypic variation where one or more traits are improved relative to the parental line that was not subjected to expression of a DNA. methyltransferase fusion protein can be selected. Useful phenotypic variation that can be selected in such individual progeny lines includes, but is not limited to, increases in fresh and dry weight biomass and/or yield relative to the parental line.

In certain embodiments, an outcross of an individual line exhibiting discrete epigenetic variability can be to a plant that has not been subjected to expression of a DNA methyltransferase fusion protein but is otherwise isogenic to the individual line exhibiting discrete variation. In certain exemplary embodiments, a line exhibiting discrete epigenetic variation is obtained by expression of a DNA methyltransferase fusion protein in a given germplasm and outcrossing to a plant having that same germplasm that was not subjected expression of a DNA methyltransferase fusion protein. In other embodiments, an outcross of an individual line exhibiting discrete epigenetic variability can be to a plant that has not been subjected to expression of a DNA methyltransferase fusion protein but is not isogenic to the individual line exhibiting discrete epigenetic variation. In other embodiments, an outcross of an individual line exhibiting discrete epigenetic variability can be to a plant that has been subjected to expression of a DNA methyltransferase fusion protein but is isogenic or is not isogenic to the individual line exhibiting discrete epigenetic variation. Thus, in certain embodiments, an outcross of an individual line exhibiting discrete epigenetic variability can also be to a plant that comprises one or more chromosomal or epigenetic polymorphisms that do not occur in the individual line exhibiting discrete epigenetic variability, to a plant derived from partially or wholly different germplasm, or to a plant of a different heterotic group (in instances where such distinct heterotic groups exist). It is also recognized that such an outcross can be made in either direction. Thus, an individual line exhibiting discrete variability can be used as either a pollen donor or a pollen recipient to a plant that has not been subjected to expression of a DNA methyltransferase fusion protein in such outcrosses. In certain embodiments, the progeny of the outcross are then selfed to establish individual lines that can be separately screened to identify lines with improved traits relative to parental lines. Such individual lines that exhibit the improved traits are then selected and can be propagated by further selfing

In certain embodiments, sub-populations of plants comprising the useful traits and epigenetic changes induced by expression of a DNA methyltransferase fusion protein can be selected and bred as a population. Such populations can then be subjected to one or more additional rounds of selection for the useful traits and/or epigenetic changes to obtain subsequent sub-populations of plants exhibiting the useful trait and/or epigenetic changes. Any of these sub-populations can also be used to generate a seed lot. In an exemplary embodiment, plants subjected to expression of a DNA methyltransferase fusion protein and exhibiting a useful or distinct phenotype can be selfed or outcrossed to obtain an F1 generation. A bulk selection at the F1, F2, and/or F3 generation can thus provide a population of plants exhibiting the useful trait and/or epigenetic changes and/or a seed lot. In certain embodiments, it is also anticipated that populations of progeny plants or progeny seed lots comprising a mixture of inbred and/or hybrid germplasms can be derived from populations comprising hybrid germplasm (i.e. plants arising from cross of one inbred line to a distinct inbred line). Seed lots thus obtained from these exemplary method or other methods provided herein can comprise seed wherein at least 25%-50%, 50%-70%, 70%-80%, 80%-90%, 90%-95%, or 95% -100% of progeny plants grown from the seed exhibit a useful trait to a greater extent than control plants. The selection would provide the most robust and vigorous of the population for seed lot production, Seed lots produced in this manner could be used for either breeding or sale. In certain embodiments, a seed lot comprising seed wherein at least 25%-50%, 50%-70%, 70%-80%, 80%-90%, 90%-95%, or 95%-100% of progeny plants grown from the seed exhibit a useful trait associated with one or more epigenetic changes, wherein the epigenetic changes are associated with CG hyper-methylation and/or CHG andlor CHH hyper-methylation at one or more nuclear chromosomal loci, preferably including, but not limited to, pericentrometic regions and transposable elements, in comparison to a control plant that does not exhibit the useful trait_{;} and wherein the seed or progeny plants grown from said seed that is epigenetically heterogenous are obtained: A seed lot obtainable by these methods can include at least 1-100, 100-500, 500-1000, 1000-5000, 5,000-10,000, 10,000-1,000,000 or more seeds.

Targeted chromosomal loci that can confer at least one useful trait can also be identified and selected by performing appropriate comparative analyses of reference plants that do not exhibit the useful traits and test plants obtained from a parental plant or plant cell that had been subjected to expression of a DNA methyltransferase fusion protein. It is anticipated that a variety of reference plants and test plants can be used in such comparisons and selections. In certain embodiments, the reference plants that do not exhibit the useful trait include, but are not limited to, any of: a) a wild-type plant; b) a distinct subpopulation of plants within a given F2 population of plants of a given plant line (where the F2 population is any applicable plant type or variety); c) an F1 population exhibiting a wild type phenotype (where the F1 population is any applicable plant type or variety); and/or, d) a plant that is isogenic to the parent plants or parental cells of the test plants prior to expression of a DNA methyltransferase fusion protein in those parental plants or plant cells (i.e. the reference plant is isogenic to the plants or plant cells that were later subjected to expression of a DNA methyltransferase fusion protein to obtain the test plants). In certain embodiments, the test plants that exhibit the useful trait include, but are not limited to, any of: a) any non-transgenic segregants that exhibit the useful trait and that were derived from parental plants or plant cells that had been subjected to expression of a DNA methyltransferase fusion protein, b) a distinct subpopulation of plants within a given F2 population of plants of a given plant line that exhibit the useful trait (where the F2 population is any applicable plant type or variety); (c) any progeny plants obtained from the plants of (a) or (b) that exhibit the useful trait; or d) a plant or plant cell that had been subjected to expression of a DNA methyltransferase fusion protein that exhibit the useful trait.

In certain embodiments, DNA methylation of targeted chromosomal loci can be identified by identifying small RNAs that are up or down regulated in the test plants (in comparison to reference plants). This method is based in part on identification of small interfering RNAs that direct or maintain DNA methylation of specific gene targets by RNA-directed DNA methylation (RdDM). The RNA-directed DNA methylation (RdDM) process has been described (Chinnusamy V et al. Sci China Ser C-Life Sci.. (2009) 52(4): 331-343). Any applicable technology platform can be used to compare small RNAs in the test and reference plants, including, but not limited to, microarray-based methods (Franco-Zorilla et al. Plant J. 2009 59(5):840-50); deep sequencing based methods (Wang et al. The Plant. Cell 21:1053-1069 (2009)); and the like. Any applicable technology platform can be used to compare small RNAs in the test and reference plants, including, but not limited to: microarray-based methods (Franco-Zorilla et al. Plant J. 200959(5):840-50); deep sequencing based methods (Wang et al. The Plant Cell 21:1053-1069(2009); Wei et al., Proc Natl Acad Sci USA. 2014 Feb 19, 111(10): 3877-3882; Zhai et al., Methods. 2013 Jun 28. pii: S1046-2023(13)00237-5. doi: 10.1016/j.ymeth.2013.06.025 or j. Zhai et al., Methods (2013), http://dx.doi.org/10.1016/j.ymeth.2013.06.025), U.S. Pat. Nos. 7,550,583; 8,399,221; 8,399,222; 8,404,439; 8,637,276; Rosas-Cardenas et al., (2011) Plant Methods 2011, 7:4; Moyano et al, BMC Genomics. 2013 Oct 11;14:701; Eldem et al., PLoS One. 2012;7(12):e50298; Barber et al., Proc Nati Acad Sci U S A. 2012 Jun 26;109(26):10444-9; Gommans et al., Methods Mol Biol. 2012;786:167-78; and the like.

DNA methylation and sRNAs corresponding to methylated DNA regions can change in progeny plants when two parent plants are crossed. Tomato progeny plants from a cross displayed transgressive sRNAs that were more abundant in the progeny than in either parent (Shivaprasad et al., EMBO J. 2012 Jan 18;31(2):257-66). A cross between two maize lines, B73 and Mo17, yielded paramutation type switches of the DNA methylation pattern of one parent chromosome being switched to that of the other parental chromosome at the corresponding loci (Regulski et al., Genome Res. 2013 Oct;23(10):1651-62). A cross between Arabidopsis plants produced progeny wherein the DNA methylation patterns of one parental chromosome were imposed onto the other parental chromosome, either gaining or losing DNA methylation levels (Greaves et al., Proc Natl Acad Sci USA. 2014 Feb 4;111(5):2017-22). These non-limiting examples indicate DNA methylation patterns can be more complex than just additive patterns from both parents. Accordingly, an objective is to produce new patterns of DNA methylation and/or of sRNA profiles. New combinations can result both from genetic segregation of targeted chromosomal loci in the progeny as well as due to changes in DNA methylation and sRNA profiles due to transgressive, paramutation type switching, and other biological processes. In certain embodiments, targeted chromosomal loci are derived from a parental plant subjected to expression of a DNA methyltransferase fusion protein. In certain embodiments, altered chromosomal loci are derived from the formation of new patterns of DNA methylation and sRNA levels from the interaction of targeted chromosomal loci derived from a parental plant subjected to expression of a DNA methyltransferase fusion protein with chromosomal loci from a second plant. Said second plant can be from a parental plant subjected to suppression of MSH1 or expression of a DNA methyltransferase fusion protein or from a parental plant not subjected to suppression of MSH1 or expression of a DNA methyltransferase fusion protein. In certain embodiments, crossing parental lines both previously subjected to expression of a DNA methyltransferase fusion protein and containing different groupings of targeted chromosomal loci provides a method of creating new combinations of targeted chromosomal loci.

Any applicable technology platform can be used to compare the DNA methylation status of targeted chromosomal loci in the test and reference plants. Applicable technologies for identifying chromosomal loci with changes in their methylation status include, but not limited to, methods based on immunoprecipitation of DNA with antibodies that recognize 5-methylcytidine, methods based on use of methylation dependent restriction endonucleases and PCR such as McrBC-PCR methods (Rahinowicz, et al. Genome Res. 13: 2658-2664 2003; Li et al., Plant Cell 20:259-276, 2008), sequencing of bisulfite-converted DNA (Frommer et al. Proc. Natl. Acad. Sci. U.S.A. 89 (5): 1827-31; Tost et al. BioTechniques 35 (1): 152-156,2003), methylation-specific PCR analysis of bisulfite treated DNA (Herman et al. Proc. Natl. Acad. Sci. U.S.A. 93 (18): 9821-6, 1996), deep sequencing based methods (Wang et al. The Plant Cell 21:1053-1069 (2009)), methylation sensitive single nucleotide primer extension (MsSnuPE; Gonzalgo and Jones Nucleic Acids Res. 25 (12): 2529-2531, 1997), fluorescence correlation spectroscopy (Umezu et al. Anal Biochem. 415(2):145-50, 2011), single molecule real time sequencing methods (Flusberg et al. Nature Methods 7,461-465), high resolution melting analysis (Wojdacz and Dobrovic (2007) Nucleic Acids Res. 35 (6): e41), and the like.

Additional applicable technologies for identifying chromosomal loci with changes in their DNA methylation status include, but not limited to, the preparation, amplification and analysis of Methylome libraries as described in U.S. Pat. No. 8,440,404; using Methylation-specific binding proteins as described in U.S. Pat. No. 8,394,585; determining the average DNA methylation density of a locus of interest within a population of DNA fragments as described in U.S. Pat. No. 8,361,719; by methylation-sensitive single nucleotide primer extension (Ms-SNuPE), for determination of strand-specific methylation status at cytosine residues as described in U.S. Pat. No. 7,037,650; a method for detecting a methylated CpG-containing nucleic acid present in a specimen by contacting the specimen with an agent that modifies unmethylated cytosine and amplifying the CpG-containing nucleic acid using CpG-specific oligonucleotide primers as described in U.S. Pat. No. 6,265,171; an improved method for the bisulfite conversion of DNA for subsequent analysis of DNA methylation as described in U.S. Pat. No. 8,586,302; for treating genomic DNA samples with sodium bisulfite to create methylation-dependent sequence differences, followed by detection with fluorescence-based quantitative PCR techniques as described in U.S. Pat. No. 8,323,890; a method for retaining methylation pattern in globally amplified DNA as described in U.S. Pat. No. 7,820,385; a method for detecting cytosine methylations DNA as described in U.S. Pat. No. 8,241,855; a method for quantification of methylated DNA as described in U.S. Pat. No. 7,972,784; a highly sensitive method for the detection of cytosine methylation patterns as described in U.S. Pat. No. 7,229,759; additional methods for detecting DNA methylation changes are described in U.S. Pat. No. 7,943,308 and U.S. Pat. No. 8,273,528.

In still other embodiments, DNA methylation at CCA1 and/or LHY promoters can be introduced by expression of a siRNA or hairpin RNA or Pol IV/Pol V recruitment method (Johnson et al., Nature. 2014 Mar 6;507(7490):124-8), targeted to CCA1 and/or LHY promoters by this method of RNA directed DNA methylation (Chinnusamy V et al. Sci China Ser C-Life Sci. (2009) 52(4): 331-343; Cigan et al. Plant J 43 929-940, 2005; Heilersig et al. (2006) Mol Genet Genomics 275 437-449; Mild and shinamoto, Plant Journal 56(4):539-49; Okano et al. Plant Journal 53(1):65-77, 2008).

In still other embodiments, CRISPR/CAS9 systems or other gene replacement methods such as TALEN-nucleases, zinc finger-guided nucleases, meganucleases are used for genome editing to create DNA methyltransferase fusion proteins in endogenous genes (Strauβ and Lahaye, Mol Plant. 2013 Sep;6(5):1384-7),

Exemplary promoters useful for expression of transgenes, including expression of a DNA methyltransferase fusion protein, include, but are not limited to, singular, enhanced or duplicated versions of the viral CaMV35S and FMV35S promoters (U.S. Pat. No. 5,378,619), the cauliflower mosaic virus (CaMV) 19S promoters, the rice Acti promoter and the Figwort Mosaic Virus (FMV) 35S promoter (U.S. Pat. No. 5,463,175). Exemplary introns useful for transgene expression include, but are not limited to; the maize hsp70 intron (U.S. Pat. No. 5,424,412), the rice Act1 intron (MCElroy et al., 1990, The Plant Cell, Vol. 2, 163-171), the CAT-1 intron (Cazzonnelli and Velten, Plant Molecular Biology Reporter 21: 271-280, September 2003), the pKANNIBAL intron (Wesley et al., Plant J. 2001 27(6):581-90; Collier et al., 2005, Plant J 43: 449-457), the PIV2 intron (Mankin et al. (1997) Plant Mol. Biol. Rep. 15(2): 186-196) and the “Super Ubiquitin” intron (U.S. Pat. No. 6,596,925; Collier et al., 2005, Plant J 43: 449-457). Exemplary 3′ polyadenylation sequences include, but are not limited to, the Agrobacterium tumor-inducing (Ti) plasmid nopaline synthase (NOS) gene 3′ potyadenylation region; the CaMV 35S 3′ polyadenylation region, the OCS 3′ polyadenylation region, and the pea RUBISCO E9 gene 3′ polyadenylation sequences.

Plant lines and plant populations obtained by the methods provided herein can be screened and selected for a variety of useful traits by using a wide variety of techniques. In particular embodiments provided herein, individual progeny plant lines or populations of plants obtained from the selfs or outcrosses of plants subjected to expression of a DNA methyltransferase fusion protein to other plants are screened and selected for the desired useful traits. In certain embodiments, the screened and selected trait is improved plant yield. In certain embodiments, such yield improvements are improvements in the yield of a plant line relative to one or more parental line(s) under non-stress conditions. Non-stress conditions comprise conditions where water, temperature, nutrients, minerals; and light fall within typical ranges for cultivation of the plant species. Such typical ranges for cultivation comprise amounts or values of water, temperature, nutrients, minerals, and/or light that are neither insufficient nor excessive. In certain embodiments, such yield improvements are improvements in the yield of a plant line relative to parental line(s) under abiotic stress conditions. Such abiotic stress conditions include, but are not limited to, conditions where water, temperature, nutrients, minerals, and/or light that are either insufficient or excessive. Abiotic stress conditions would thus include, but are not limited to, drought stress, osmotic stress, nitrogen stress, phosphorous stress, mineral stress, heat stress, cold stress, and/or light stress. In this context, mineral stress includes, but is not limited to, stress due to insufficient or excessive potassium, calcium, magnesium, iron, manganese, copper, zinc, boron, aluminum, or silicon. In this context, mineral stress includes, but is not limited to, stress due to excessive amounts of heavy metals including, but not limited to, cadmium, copper, nickel, zinc, lead, and chromium.

Improvements in yield in plant lines obtained by the methods provided herein can be identified by direct measurements of wet or dry biomass including, but not limited to, grain, lint, leaves, stems, or seed. Improvements in yield can also be assessed by measuring yield. related traits that include, but are not limited to, 100 seed weight, a harvest index, and seed weight. In certain embodiments, such yield improvements are improvements in the yield of a plant line relative to one or more parental line(s) and can be readily determined by growing plant lines obtained by the methods provided herein in parallel with the parental plants. In certain embodiments, field trials to determine differences in yield whereby plots of test and control plants are replicated, randomized, and controlled for variation can be employed (Giesbrecht F G and Gumpertz M L 2004. Planning, Construction, and Statistical Analysis of Comparative Experiments Wiley. New York; Mead, R. 1997. Design of plant breeding trials. In Statistical Methods for Plant Variety Evaluation eds. Kempton and Fox. Chapman and Hall. London.). Methods for spacing of the test plants (i.e. plants obtained with the methods of this invention) with check plants (parental or other controls) to obtain yield data suitable for comparisons are provided in references that include, but are not limited to, any of Cullis, B. et al. J. Agric. Biol. Env. Stat.11:381-393; and Besag, J. and Kempton, R A. 1986. Biometrics 42: 231-251.).

In certain embodiments, the screened and selected trait is improved resistance to biotic plant stress relative to the parental lines. Biotic plant stress includes, but is not limited to, stress imposed by plant fungal pathogens, plant bacterial pathogens, plant viral pathogens, insects, nematodes, and herbivores. In certain embodiments, screening and selection of plant lines that exhibit resistance to fungal pathogens including, but not limited to, an Alternaria sp., an Ascochyta sp., a Botrytis sp.; a Cercospora sp., a Colletoirichum sp., a Diaporthe sp., a Diplodia sp., an Erysiphe sp., a Fusarium sp., Gaeumanomyces sp., Hehninthosporium sp., Macrophomina sp., a Nectria sp., a Peronospora sp., a Phakopsora sp., Phialophora sp., a Phoma sp., a Phymatotrichum sp., a Phytophthora sp., a Plasmopara sp., a Puccinia sp., a Podosphaera sp., a Pyrenophora sp., a Pyricularia sp, a Pythium sp., a Rhizoctonia sp., a Scerotium sp., a Sclerotinia sp., a Septoria sp., a Thielaviopsis sp., an Uncimula sp, a Venturia sp., and a Verticillium sp. is provided. In certain embodiments, screening and selection of plant lines that exhibit resistance to bacterial pathogens including, but not limited to, an Erwinia sp., a Pseudomonas sp., and a Xanthamonas sp. is provided. In certain embodiments, screening and selection of plant lines that exhibit resistance to insects including, but not limited to, aphids and other piercing/sucking insects such as Lygus sp., lepidoteran insects such as Armigera sp., Helicoverpa sp., Heliothis sp., and Pseudophisia sp., and coleopteran insects such as Diabroticus sp. is provided. In certain embodiments, screening and selection of plant lines that exhibit resistance to nematodes including, but not limited to, Meloidogyne sp., Heterodera sp., Belonolaimus sp., Ditylenchus sp., Globodera sp., Naccobbus sp., and Xiphinema sp. is provided.

Other useful traits that can be obtained by the methods provided herein include various seed quality traits including, but not limited to, improvements in either the compositions or amounts of oil, protein, or starch in the seed. Still other useful traits that can be obtained by methods provided herein include, but are not limited to, increased biomass, non-flowering, male sterility, digestability, seed filling period, maturity (either earlier or later as desired), reduced lodging, and plant height (either increased or decreased as desired).

In addition to any of the aforementioned traits, particularly useful traits that can be obtained by the methods provided herein also include, but are not limited to: i) agronomic traits (flowering time, days to flower, days to flower-post rainy, days to flowering; ii) fungal disease resistance; iii) grain related traits: (Grain dry weight, grain number, grain number per square meter, Grain weight over panicle, seed color, seed luster, seed size); iv) growth and development stage related traits (basal tillers number, days to harvest, days to maturity, nodal tillering, plant height, plant height); v) infloresence anatomy and morphology trait (threshability); vi) Insect damage resistance; vii) leaf related traits (leaf color, leaf midrib color, leaf vein color, flag leaf weight, leaf weight, rest of leaves weight); viii) mineral and ion content related traits (shoot potassium content, shoot sodium content); ix) panicle, pod, or ear related traits (number of panicles and seeds, harvest index, panicle weight); x) phytochemical compound content (plant pigmentation); xii) spikelet anatomy and morphology traits (glume co)or, glume covering); xiii) stem related trait (stem over leaf weight, stem weight); and xiv) miscellaneous traits (stover related traits, metabolised energy, nitrogen digestibility, organic matter digestibility, stover dry weight).

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyatnus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieutn, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plants or plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentalle), macadamia (Macadamia spp.), almond (Prunus amygdalus), sugar beets (Beta vulgaris), sugarcane (Saccharum spp.), oats, barley, vegetables, ornamentals, and conifers.

Examples of suitable vegetables plants may include, for example, tomatoes (Lycopersicon esculentutn), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiaptilcherrima), and chrysanthemum.

Examples of suitable ornamental plants may include, for example, azalea (Rhododendron spp.), hydrangea (Macrophlla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago s sp.), orchard grass, tall fescue, perennial ryegrass, creeping bent grass, and redtop.

In general, methods provided herewith for introducing epigenetic variation in plants require plants or plant cells to be subjected to constitutive or inducible expression of a DNA methyltransferase fusion protein for a time sufficient in whole plants or in appropriate subsets of cells, particularly med stem or reproductive cells or cell lineages. As such, a wide variety of methods of expressing a DNA methyltransferase fusion protein can be employed to practice the methods provided herewith and the methods are not limited to a particular expression technique. In certain embodiments, DNA methyltransferase fusion protein genes may be used directly in either a homologous or a heterologous plant species to provide for expression of a DNA, methyltransferase fusion protein gene in either the homologous or heterologous plant species. A transgene comprising a DNA methyltransferase fusion pro e n comprising a DNA methyltransferase from Arabidopsis or rice or other plant species or non-plant species that provides for expression of a DNA methyltransferase fusion protein can be used in certain embodiments in millet, sorghum, and maize, or other plants including, but not limited to, cotton, canola, wheat, barley, flax, oat, rye, turf grass, sugarcane, alfalfa, banana, broccoli, cabbage, carrot, cassava, cauliflower, celery, citrus, a cucurbit, eucalyptus, garlic, grape, onion, lettuce, pea, peanut, pepper, potato, poplar, pine, sunflower, safflower, soybean, strawberry, sugar beet, sweet potato, tobacco, cassava, cauliflower, celery, citrus, cotton, a cucurbit, eucalyptus, garlic, grape, onion, lettuce, pea, peanut, pepper, potato, poplar, pine, sunflower, safflower, strawberry, sugar beet, sweet potato, tobacco, cassava, cauliflower, celery, citrus, cucurbits, eucalyptus, garlic, grape, onion, lettuce, pea, peanut, pepper, poplar, pine, sunflower, safflower, soybean, strawberry, sugar beet, tobacco, Jatropha, Camelina, and Agave.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### SgRNA for CRISPR/CAS9 proteins

SgRNA for Streptococcus pyogene. A sgRNA suitable for targeting a S. pyogenes CRISPR/CAS9 protein to DNA target sites in the genome has the following design: a 17 to 20 nucleotide base-pairing region that is complementary or homologous to the target I)NA sequence, a 42 nt Cas9 recognition hairpin structure, and a 40 nt S. pyogenes terminator including a 3′ hairpin followed by poly U nt tail of 4 or more U nt) and has the general sequence shown in SEQ ID NO:1, wherein T is transcribed as U in the sgRNA., and the N20 (actually a range of N17 to N20) is the sequence of the intended target DNA. The intended target DNA sequence needs to contain a PAM sequence of NGG such that the target I)NA sequence of the genomic DNA is 5′-N20-NGG-3′. Shorter 17 to 19 nt regions of homology in the sgRNAs can be used for increased specificity (Fu, Sander et al. 2014). A related optimized sgRNA is available for Streptococcus thermophiles CRISPR/CAS9 systems (SEQ ID NO:2; (Xu, Ren et al. 2014)).

Species of Neisseria, such as Neisseria meningitides, also contain CRISPR/CAS9 systems suitable for RNA-guided DNA binding of the sgRNA-CRISPR/CAS9 protein complex (Hou, Zhang et al. 2013). Neisseria meningitides has a different adjacent PAM requirement in the host target sequence as it requires 5′-NNNNGATT downstream of the target homology (Hou, Zhang et al, 2013). Neisseria meningitides has the general sgRNA, sequence shown in SEQ ID NO:3.

### Example 2

### RNA Pol III Promoters for sgRNA transcription in plants

As used herein, “a Pol III promoter” is a promoter which directs transcription of the operably attached DNA region through transcription by RNA polymerase III. These include genes encoding 5S RNA, tRNA, 7SL RNA, U6 snRNA and a few other small stable RNAs, many involved in RNA processing. Most of the promoters used by Pol III require sequence elements downstream of +1, within the transcribed region. A minority of pol III templates however, lack any requirement for intragenic promoter elements. These are referred to as type 3 promoters. In other words, “type 3 Pol III promoters” are those promoters which are recognized by RNA polymerase III and contain all cis-acting elements, interacting with the RNA polymerase III upstream of the region normally transcribed by RNA polymerase III. Such type 3 Pol III promoters can thus easily be combined in a chimeric gene with a heterologous region, the transcription of which is desired, such as the sgRNA coding regions of the current invention. Type 3 Pol III promoters are associated with genes encoding 7SL RNA, U3 snRNA and U6 snRNA.

For dicot plants, the Arabidopsis thatiana U6-26 promoter and 3′ end region, and containing a sgRNA structure is suitable for expressing sgRNAs, wherein the first base of the transcribed sgRNA is a G nt (Mao, Zhang et al. 2013). For sgRNAs with a 5′ terminal ‘A’ nt, the Arabidopsis thaliana U3B promoter and 3′ end region, and containing a sgRNA structure is suitable for expressing sgRNAs.

For the S. pyogenes CRISPR/CAS9, the general sequence of a Arabidopsis U6-26 gene sgRNA cassette is shown in SEQ ID NO:4 with the target homology region indicated as GN(19).

For the S. thermophiles CRISPR/CAS9, the general sequence of a Arabidopsis U6-26 gene sgRNA cassette is shown in SEQ II) NO:5 with the target homology region indicated as GN(18).

For the Neisseria meningitides CRISPR/CAS9, the general sequence of a Arabidopsis U6-26 gene cassette is shown in SEQ ID NO:6 with the target homology region indicated as GN(23).

For Monoeot Plants, the Following RNA Pol III Promoters are Suitable for Expressing sgRNAs.

The maize ZmU3 promoter (Liang, Zhang et al. 2014); the rice pOsU3-sgRNA (Mao, Zhang et al. 2013; Shan, Wang et al. 2013) which initiates transcription at an ‘A’; the U6-gRNA for wheat which initiates transcription at a ‘G’(Shan, Wang et al. 2013); and two U6-sgRNA promoters for rice (Jiang, Zhou et al. 2013) have been used for generating sgRNA in plants.

Other nucleotide sequences for type 3 Pol III promoters can be found in nucleotide sequence databases under the entries for the A. thaliana gene AT7SL-1 for 7SL RNA (X72228), A. thaliana gene AT7SL-2 for 7SL RNA (X72229), A. thaliana gene AT7SL-3 for 7SL RNA (M290403), Humulus lupulus H17SL-1 gene (AJ236706), Humulus lupulus H17SL-2 gene (AJ236704), Humulus lupulus H17SL-3 gene (AJ236705), Humulus lupuus H17SL-4 gene (AJ236703), A. thaliana U6-1 snRNA gene (X52527), A. thaliana U6-26 snRNA gene (X52528), A. thaliana U6-29 snRNA gene (X52529), A. thaliana U6-1 snRNA gene (X52527), Zea mays U3 snRNA gene (Z29641), Solanum tuberosum U6 snRNA gene (Z17301; X 60506; S83742), Tomato U6 smal nuclear RNA gene (X51447), A. thaliana U3C snRNA gene (X52630), A. thaliana U3B snRNA gene (X52629), Oryza saliva U3 snRNA promoter (X79685), Tomato U3 smal nuclear RNA gene (x14411), Triticum aestivum U3 snRNA gene (X63065), Triticum aestivum U6 snRNA gene (X63066).

sgRNA Genomic Targets

sgRNAs with 17, 18, 19, 20 or 21-24 at of homology to a target DNA are effective for targeting CRISPR/CAS9 complexes. The shorter 17 or 18 nt homology regions have fewer off-target sites (Fu, Sander et al. 2014). The existence of off-target effects demonstrates that target homologies can contain mismatches of up to five mismatches (Fu, Foden et al. 2013). Mismatches can be intentionally introduced into the targeting region of sgRNAs for increased specificity whereby the mismatches are chosen to have a targeting region with less homology to off-target regions in the genome when computationally analyzed for off-target sites. Many such computational programs are known to those skilled in the art. Expression of multiple sgRNAs is most readily accomplished from an array of multiple sgRNA gene cassettes, with examples of two (Mao, Zhang et al. 2013), three (Ma, Chang et al. 2014), four (Perez-Pinera, Kocak et al. 2013; Ma, Shen et al. 2014), five (Jao, Wente et al. 2013), six (Liu et al., Insect Biochem Mol Biol. 2014 Jun;49:35-42), or seven sgRNAs (Sakuma, Nishikawa et al. 2014). One or more of the RNA Pol III gene cassettes available for expressing sgRNAs can be used in an array of two or more gene cassettes to express multiple sgRNAs.

### Example 3

### CRISPR CAS9 Proteins as DNA Binding Proteins

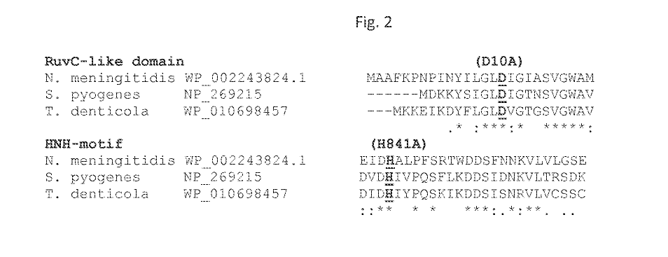

CRISPR/CAS9 proteins that bind guide RNA.(s) for RNA-guided DNA binding and endonuclease activity are widely distributed in bacterial species. In the three Streptococcus, Neisseria. Treponema genera demonstrated to provide CRISPR″CAS9 gene targeting in eukaryotes, many individual CRISPR/CAS9 protein sequences are known within each genus and display conserved protein sequences as indicated in clustal omega alignments for: Streptococcus, Neisseria, and Treponema species (FIG. 1). The RuvC-like domain and HNH-motif catalytic domains are highly conserved, particularly the D10 and H841 amino acid positions (FIG. 2). Mutation of D10A and H841A of Streptococcus pyogenes CRISPR/CAS9 produces a protein capable of RNA-guided DNA binding but lacking DNA endonuclease activity (Jinek, Chylinski et al. 2012). Alignment of Streptococcus, Neisseria, Treponema CRISPR/CAS9 proteins near the N-terminal RuvC-like domain and HNH-motif domain indicate the D10 and H841 amino acids are conserved and changing these amino acids to the D10A and H841A mutations will inactivate the nuclease activity of these classes of CRISPR/CAS9 proteins (Jinek, Chylinski et al. 2012).

CRISPR/CAS9 protein activities in eukaryotic cells benefit from containing added nuclear localization signals (NLS) such as the SV40 NLS. Synthetic CRISPR/CAS9 genes containing NLS signals at their N and/or C-termini, and wherein plant preferred codons are used to encode the protein have been demonstrated to have CRISPR/CAS9 activity in plants and animals. Three plant-preferred codon synthetic coding regions encoding Streptococcus pyogenes CRISPR/CAS9 proteins are described in (Jiang, Zhou et al. 2013) and are representative of useful CRISPR/CAS9 protein synthetic coding regions. Conversion of CRISPR/CAS9 coding regions to encode the D10A and H841A mutations that inactivate the nuclease domains is useful for producing RNA-guided DNA binding CRISPR/CAS9 proteins lacking endonuclease activity.

### Example 4

### Conserved Amino Acids in the Met1, CMT2, CMT3, and DRM2 Domains

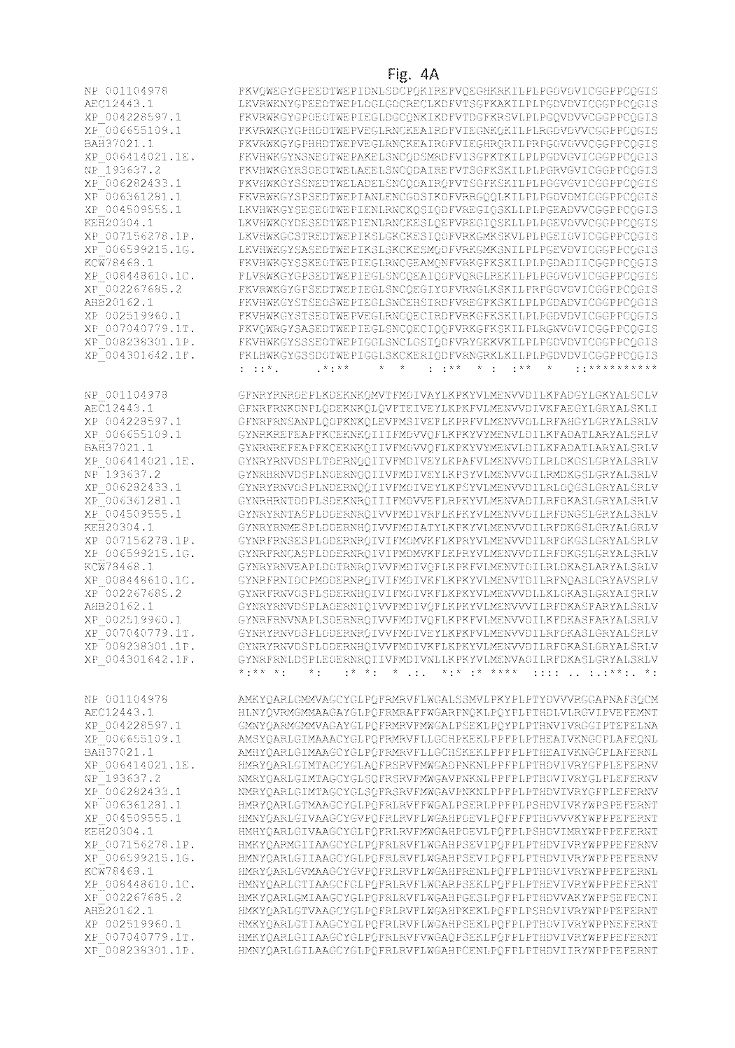

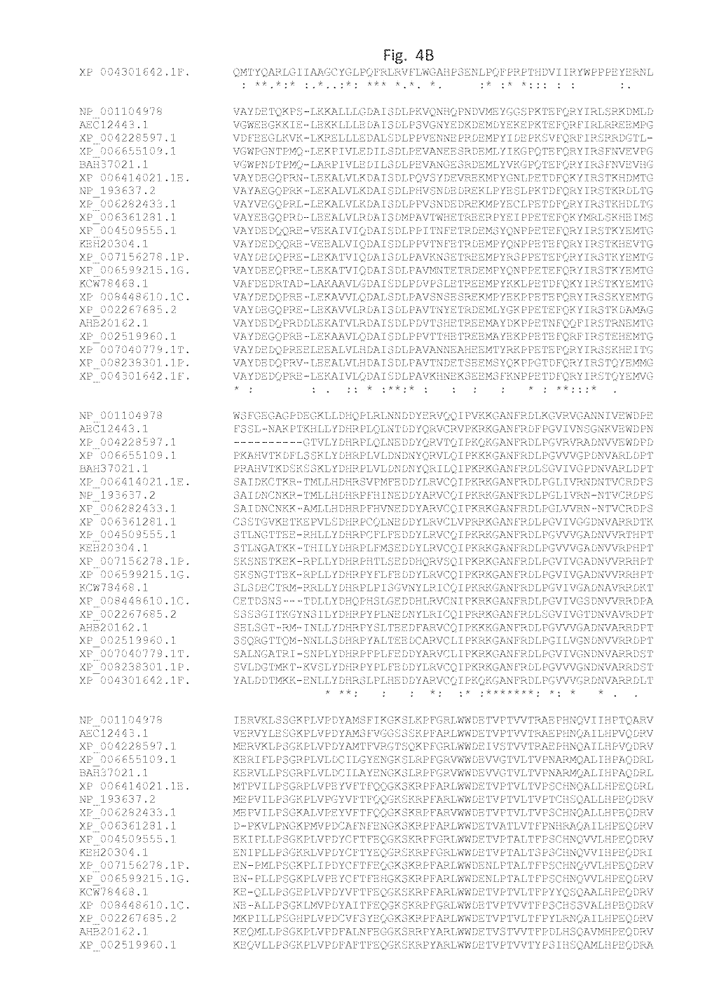

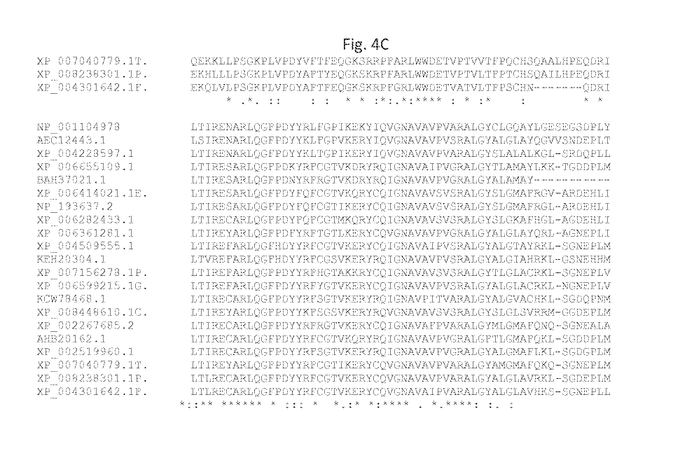

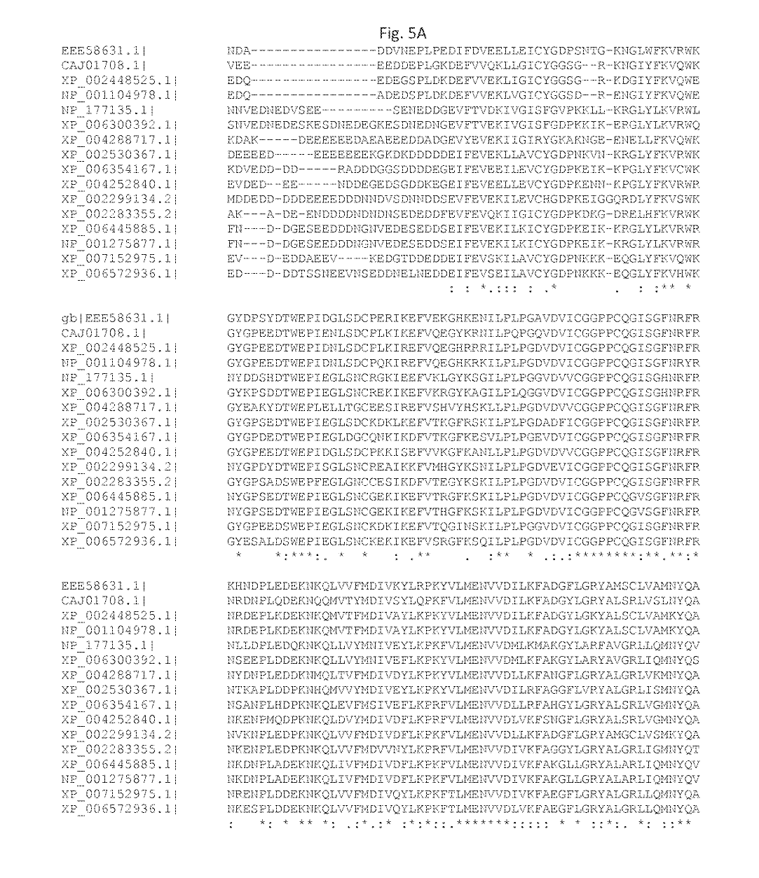

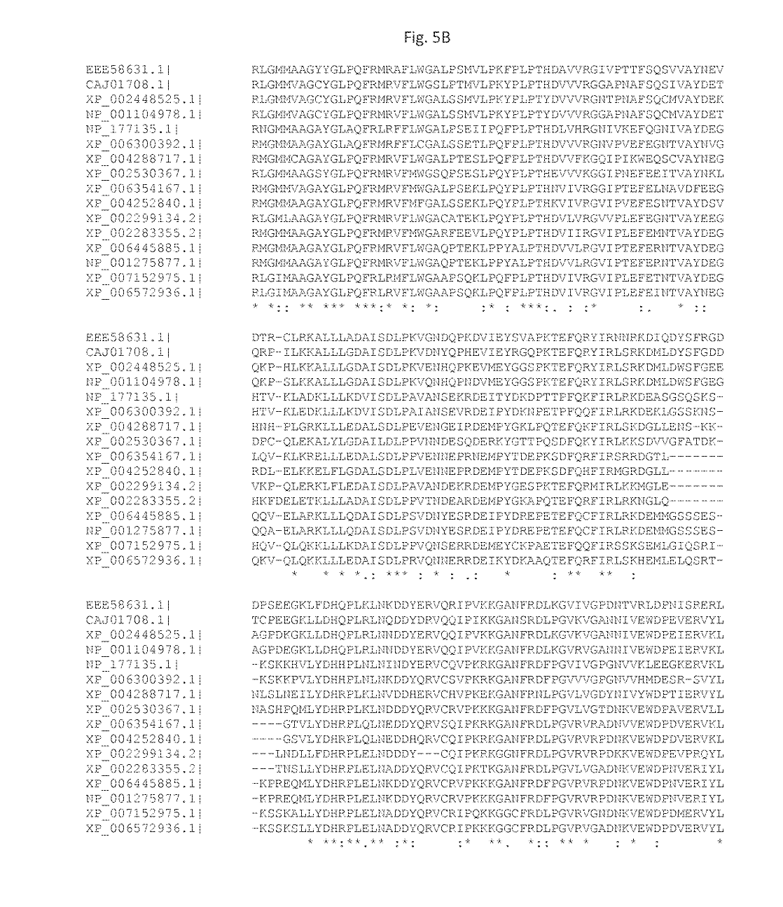

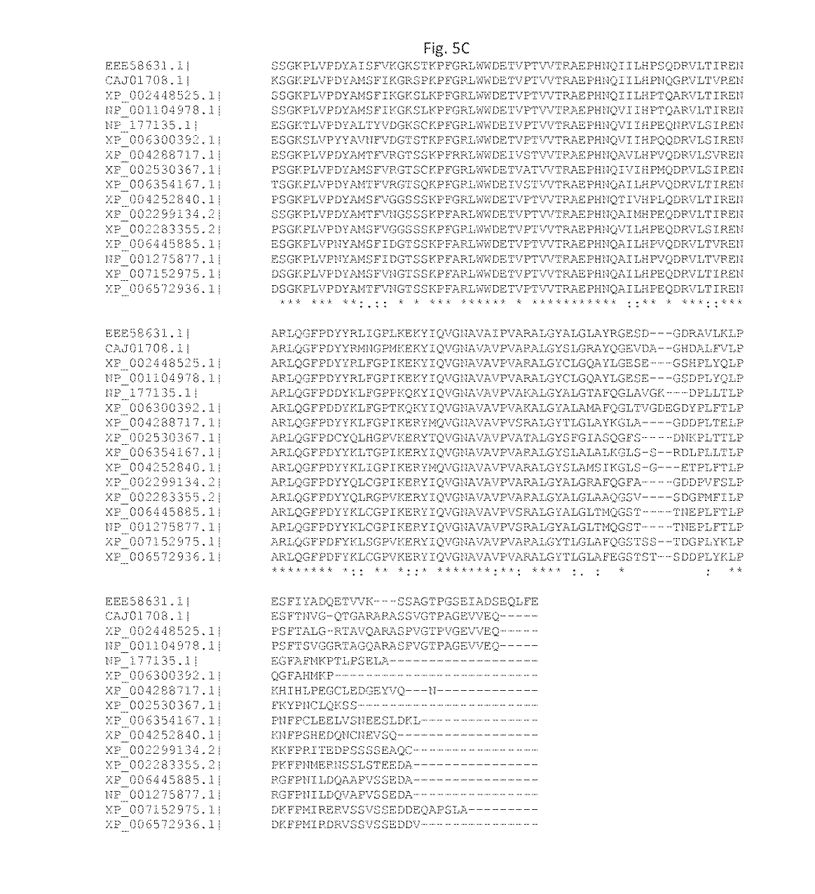

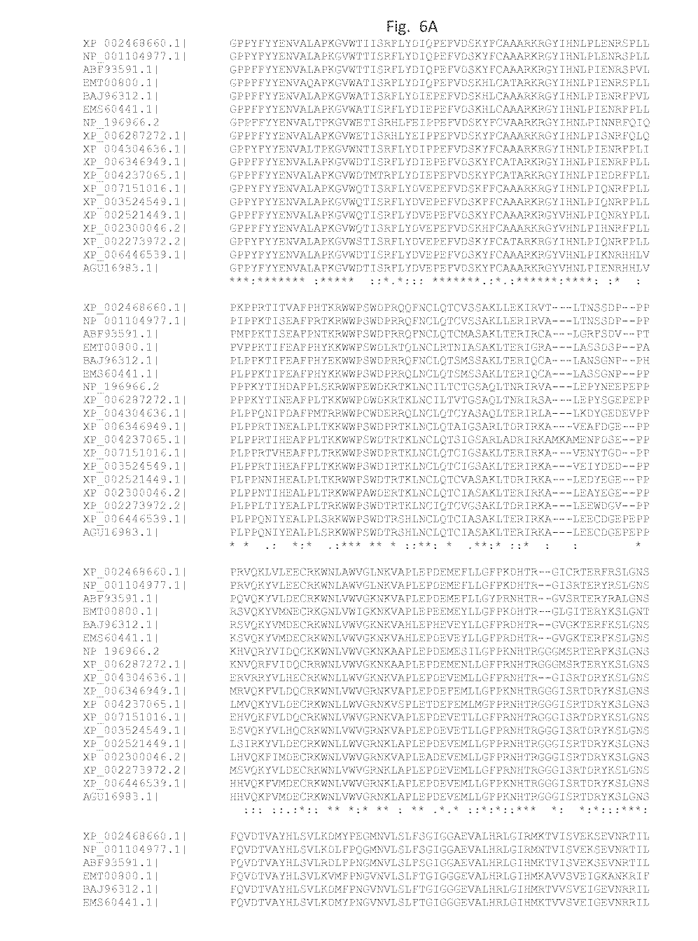

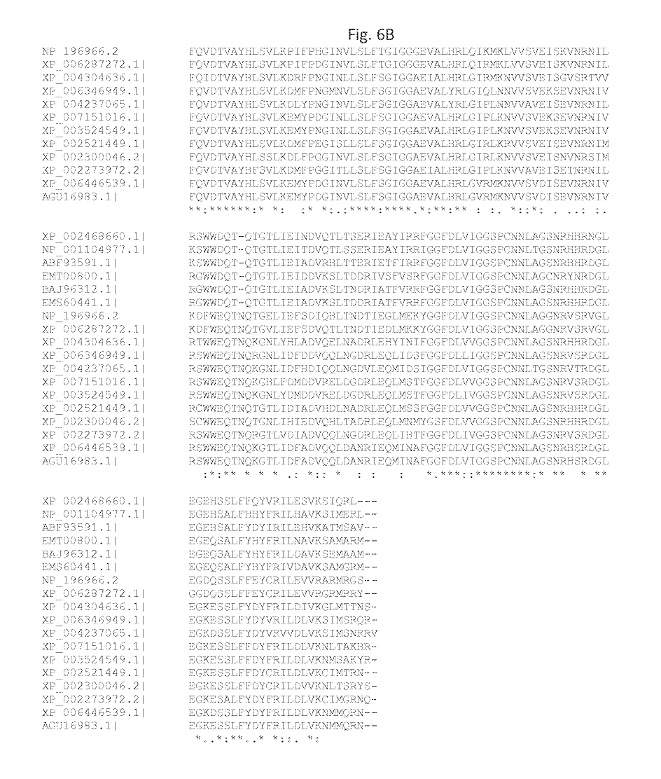

Plant DNA methyltransferases can methylate CHH and CHG, as well as CG positions, with somewhat different specificities for the different methyltransferases, Plant DNA. methyltransferases include (using Arabidopsis nomenclature) the Met1/2, CMT1/2/3, and DRM1/2 families. Members of these families can be identified in many plant species by BLAST analysis of sequences or experimentally. A non-limiting Clustal Omega analysis of the Met1 (FIG. 3), CMT2 family (FIG. 4), CMT3 family (FIG. 5), and DRM2 family (FIG. 6) indicates the sequences and conserved amino acids at equivalent positions in the more conserved C-terminal domains containing most or all of the catalytic domain of these proteins. These FIGS. 3-6 indicate the identical amino acids and some of the evolutionarily selected amino acid variations at each position of these proteins. As these proteins are functional in plants, the range of amino acids at each equivalent position indicates which amino acids can be functionally substituted at each amino acid position without disrupting protein function. Conservatively modified variants changes in proteins are also generally tolerated, indicating DNA methyltransferases containing these evolutionarily selected or conservatively modified variant amino acid differences from the protein sequences in FIGS. 3-6 are generally functional and useful for the present invention.

### Example 5

### Targeting Two CCA1-like Promoters in Soybeans with a CRS1PR/CAS9-Soybean Full Length Dian Fusion Protein in Soybeans

In this exemplary non-limiting example, two Arabidopsis U3B gene cassettes are used to express 2 separate sgRNAs, each with targeting homology against identical regions in two related CCM-like gene promoters in soybeans. The basic binary vector used for plant transformation herein is pCAMBIA1300-BAR (FIG. 7; SEQ ID NO:7), a pCAMBIA1300 derived vector that is modified to replace the hygromycin selectable marker with a Streptomyces hygroscopicus bar gene for selection of transformed plant cells with bialophos or phosphinothricin. The pCAMBIA1300-BAR binary plasmid has the BAR selectable gene as a CaMV35S promoter/BAR/CaMV 35S terminator (polyadenylation site) cassette for use as a selectable marker in plants.

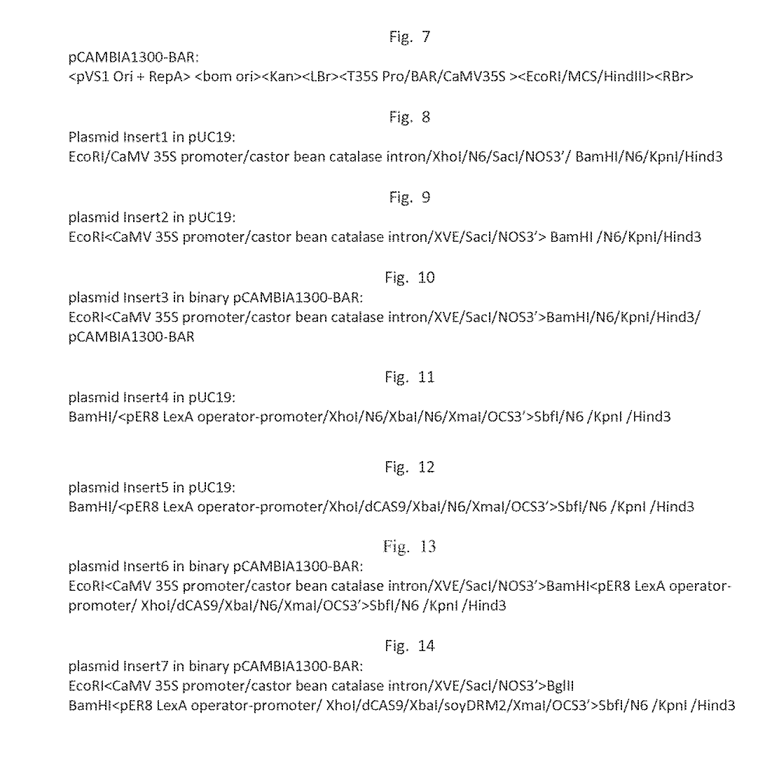

A EcoRI/CaMV 35S promoter/castor bean catalase intron/XhoI/N6/SacI/NOS3′/BamHI/N6/KpnI/Hind3 gene cassette is commercially synthesized (SEQ ID NO:9), digested with EcoRI and HindIII, purified, and ligated into similarly treated pUC19 to form plasmid Insert1 (FIG. 8). An ecdysone receptor construct similar to that of (Yang, Ordiz et al. 2012) consisting of 5′-SalI/LexA binding domain/VP16 activation domain/Ecdysone receptor domains/SacI (XVE) is commercially synthesized (XVE CDS; SEQ ID NO:10), digested with Sail and SacI restriction enzymes, purified, and ligated into a XhoI and SacI digested and purified plasmid insert1. The resulting plasmid Insert2 (FIG. 9) has the following order of elements in pUC19: EcoRI/CaMV 35S promoter/castor bean catalase intron/XVE/SacI/NOS3′/BamHI/N6/KpnI/Hind3. The insert of plasmid Insert2 is excised by digestion with EcoRI and HindIII, purified, and ligated into similarly digested and purified pCAMBIA1300-BAR to form binary plasmid Insert3 (FIG. 10).

The LexA operator/CaMV 35S minimal promoter sequence of inducible plasmid pER8, which is regulated by a chimeric LexA/VP16/estrogen receptor (Zuo, Niu et al. 2000) similar to the XVE chimeric ecdysone receptor is utilized herein for an inducible promoter cassette. The LexA operator/minimal promoter sequence of pER8 that is inducible by XVE is commercially synthesized as part of a larger commercially synthesized DNA fragment to have the following order of DNA elements: 5 BamHI/LexA operator/CaMV 35S minimal promoter from pER8/XhoI/N6/XbaI/N6/XmaI/OCS3′/SbfI/N6/KpnI/Hind3 (SEQ ID NO:12) and cloned into BamHI and HindIII digested and purified pUC19 to form plasmid Insert4 (FIG. 11).

A XhoI/NLS-dCAS9/XbaI synthetic S. pyogenes CRISPR/CAS9 coding sequence derived from a CRISPR/CAS9 sequence published by (Jiang, Zhou et al. 2013) is commercially synthesized using plant preferred codons, except for the following changes: two SV40 nuclear localization signals are placed at the N-terminus and none are at the C-terminus; a SbfI site is removed by a silent codon change; that the D10A and H841A mutations are included to inactivate its endonuclease activity; and the stop codon is removed to use this protein as a fusion protein (SEQ ID NO:13). This endonuclease inactive S. pyogenes CRISPR/1CAS9 (dCAS9) coding sequence is digested with XhoI and XbaI, purified, and ligated into XhoI and XbaI digested plasmid Insert4 to form plasmid Insert5 (FIG. 12) with the following order of elements: 5′ BamHI/LexA operator/promoter/XhoI/dCAS9/XbaI/N6/XmaI/OCS3′/SbfI/N6/KpnI/Hind3. The insert of plasmid Insert5 is excised by digestion with BamHI and KpnI, purified, and ligated into similarly digested and purified plasmid Insert3 to form plasmid Insert6 (FIG. 13) containing the following order of elements in binary plasmid pCAMBIA1300-BAR: EcoRI/CaMV 35S promoter/castor bean catalase intron/XVE CDS/SacI/NOS3′/BamHI/LexA operator/promoter/XhoI/dCAS9/XbaI/N6/XmaI/OCS3′/SbfI/N6 /KpnI/Hind3.

An XbaI/synthetic full length soy DRM2 DNA methyltransferase (soyDRM2) coding region/XmaI DNA fragment is commercially synthesized (SEQ ID NO:15), digested with XbaI and XmaI, purified, and ligated into similarly digested and purified plasmid Insert6 to form binary plasmid Insert:7 (FIG. 14) with the following order of DNA elements: EcoRI/CaMV 35S promoter/castor bean catalase intron/XVE/SacI/NOS3′/BamHI/LexA operator/promoter/XhoI/dCAS9/XbaI/soyDRM2/XmaI/OCS3′/SbfI/N6/KpnI/Hind3. The dCAS-soyDRM2 DNA methyltransferase is expressed as an inducible fusion protein from this vector in plants.

Promoter Region Target Sequences for sgRNA Design

Analysis of the soybean genome in the publically available databases (e.g., GmGDB portion of Plant GDB) identified 4 CCA1/LHY-like genes, with two pairs being more similar to each other: 2 CCA1-like (Glyma19g45030 and Glyma03g42260) and 2 LHY-like (Glyma16g01980 and Glyma07g05410). BLAST alignment of the two CCA1-like promoters (Glyma19g45030 and Glyma03g42260) or two LHY-like promoters (Glyma16g01980 and Glyma07g05410) with each other identified two identical conserved regions useful for targeting each promoter pair (CCA1-like or LHY-like) with a single sgRNA (FIG. 15).

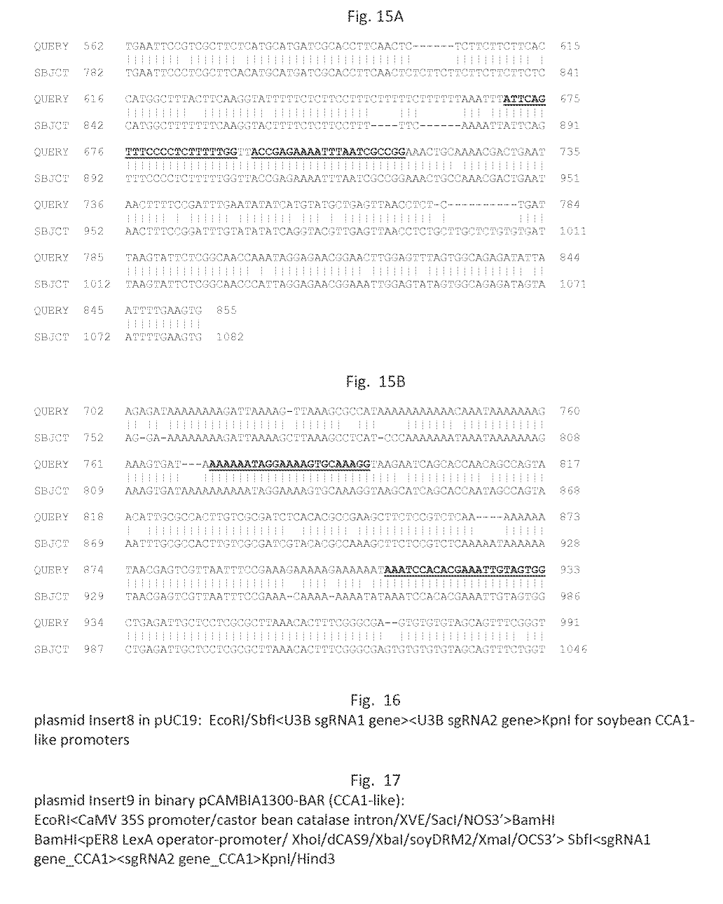

A Golden Gate BsaI Assembly method (Weber, Gruetzner et al. 2011) is used to assemble a tandem array of two commercially synthesized sgRNA gene cassettes that use the Arabidopsis U3B (AT5G53902) sequence gene cassette framework (SEQ ID NO:17). Two sgRNAs, each with a unique N19 targeting sequence with homology against two soybean CCA-like promoters (Glyma19g45030 and Glyma03g42260) were designed. The targeted sequences are identical in the two promoters, allowing for each sgRNA to target both promoter (FIG. 15). The assembled two-gene sgRNA array is flanked by SbfI and KpnI restriction sites (SEQ ID NO:18). The assembled sequence in pUC 19 in plasmid insert8 (FIG. 16) has the following elements: EcoRI/SbfI/sgRNA1 gene/sgRNA2 gene/KpnI (SEQ ID NO:18). The sgRNA insert of plasmid insert8 is excised with SbfI and KpnI, purified, and ligated to similarly digested plasmid Insert7 to form plasmid Insert9 (FIG. 17; SEQ ID NO:19) with the following DNA elements: EcoRI/CaMV 35S promoter/castor bean catalase intron/XVE CDS/SacI/NOS3′/BamHI/LexA operator/promoter/XhoI/dCAS9/XbaI/DNA Methyltransferase/XmaI/OCS3′/SbfI/sgRNA1 gene/sgRNA2 gene/KpnI/Hind3. Plasmid Insert9 has all the genetic components required for inducible targeted DNA methylation: A binary plasmid suitable for plant transformation carrying a chemically inducible XVE protein that activates transcription of dCAS9-soyDRM2, which binds sgRNA1 or sgRNA2, and is guided to the target site homologies by these sgRNAs to conduct DNA methylation in the region of the targeted sites.

Plasmid Insert9 is transformed into Agrobacterium tumefaciens for transformation into Thorne soybeans plants using glufosinate as the selection system as described (Zhang et a]., Plant Cell, Tissue and Organ Culture 56: 37-46, 1999). Potential transgenic soybean plants are screened for those that contain dCAS9 DNA by real time PCR analysis of isolated genomic DNA. Transgenic soybean plants in soil are watered with water containing 61 mM methoxyfenozide (Yang, Ordiz et al. 2012) to induce expression of the dCAS9-soyDRM2 cassette for various durations starting at 2, 4, 6, 8, or 10 weeks after germination and persisting until fertilization of the flowers. Induction by watering with 61 mM methoxyfenozide is also done for 1 to 10 days prior to flowering to provide different amounts of targeted DNA methylation. Progeny plants are analyzed phenotypically for CCA1 phenotypes for altered phenotypes, such as size and flowering time, due to DNA methylation-mediated suppression of the CCA1 gene to produce soybean plants with enhanced yields, relative to their parental control plants. DNA methylation analysis of lines containing the transgene, or their non-transgenic progeny, indicates the plants display enhanced DNA methylation relative to the CCA1 promoter regions of parental plant controls, and mRNA expression analysis indicates these plants have lower expression of CCA1 transcripts. If higher levels of DNA methylation are desired, inducible transgenic methyltransferase activity can be maintained in one or more progeny generations prior to its removal by segregation or crossing. Highly methylated CCA1 genes in non-transgenic (segregated) progeny lines can be used as self-pollinated lines or outcrossed. Out crossed lines can be further bred or selfed to produced enhanced yield lines.

### Example 6

### Targeting Two LHY-like Promoters in Soybeans with a CRISPR/CAS9-Soybean Full Length DRN12 Fusion Protein in Soybeans

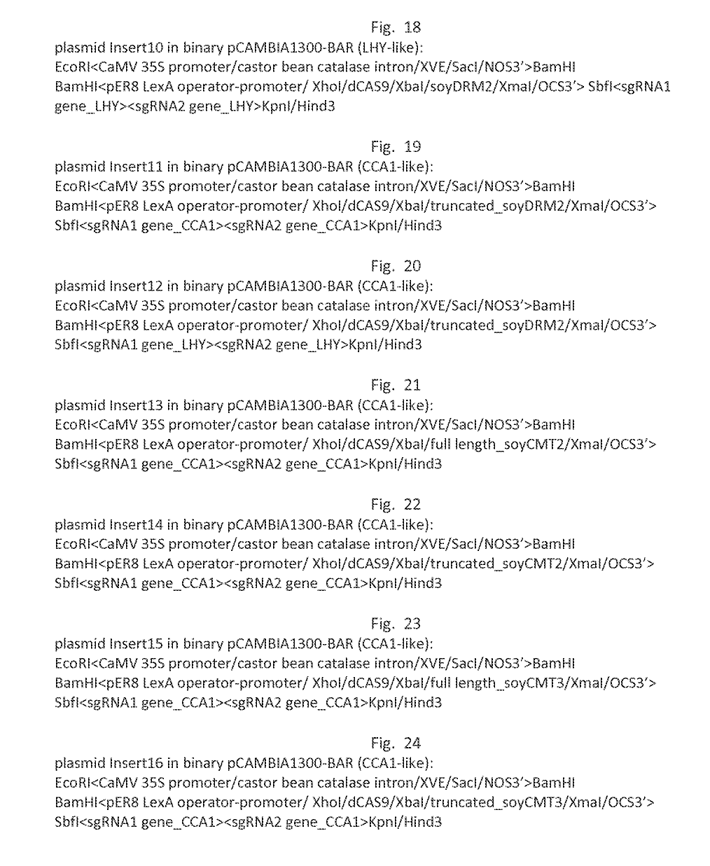

In this exemplary example, two Arabidopsis U3B gene cassettes are used to express 2 separate sgRNAs, each with targeting homology against identical regions in two related LHY-like gene promoters in soybeans, performed similarly as described in Example 5 except the target homology regions are against the two LHY-like promoters (Glyma16g01980 and Glyma07g,05410). BLAST alignment of the two LHY-like promoters (Glyma16g01980 and Glyma07g05410) identified two identical conserved regions useful for targeting both promoters, each region of each promoter being targeted with a single sgRNA (FIG. 15). The Golden Gate BsaI Assembly method (Weber, Gruetzner et al. 2011) is used to assemble a two-gene sgRNA (each commercially synthesized) array flanked by SbfI and KpnI restriction sites (SEQ ID NO:20) using the methods described in Example 5. The assembled sequence is digested with SbfI and KpnI, purified, and ligated to similarly digested plasmid Insert7 to form plasmid Insert10 (FIG. 18) with the following DNA elements: EcoRI/CaMV 35S promoter/castor bean catalase intron/XVE CDS/SacI/NOS3′/BamHI/LexA operatorlpromoter/XhoI/dCAS9/XbaI/DNA Methyltransferase/XmaI/OCS3′/SbfI/sgRNA1 gene/sgRNA2 gene/KpnI/Hind3. Plasmid Insert 10 has all the genetic components required for inducible targeted :DNA methylation: A binary plasmid suitable for plant transformation carrying a chemically inducible XVE protein that activates transcription of dCAS9-soyDRM2, which binds sgRNA1 or sgRNA2, and is guided to the target site homologies in the two LHY-like promoters by these sgRNAs to conduct DNA methylation in the region of the targeted sites. The plant transformation, breeding, and analysis are performed as described in Example 5.

### Example 7

### Crossing of the CCA1-like and LHY-like Methylation Targeted Soybean Plants

The soybean plants of Example 5 are methylation-targeted for the two CCA1-like promoters and the soybean plants of Example 6 are methylation-targeted for the two LHY-like promoters. Crossing of the two types of plants, and identifying transgenic progeny by PCR analysis of the transgenes (using the unique targeting sequences in each T-DNA are PCR primer sites) containing both types of T-DNAs allows for concurrently methylation of all four CCA1-like and Lift-like promoters in the soybean genome. Progeny plants are phenotypically analyzed and bred as described in Example 5.

### Example 8

### Targeting Two CCA1-like Promoters in Soybeans with a CRSIPR/CAS9-soybean Truncated DRM2 Fusion Protein in Soybeans

A truncated soybean DRM2 coding sequence encoding the DNA methyltransferase catalytic region of soybean DRM2 is commercially synthesized to have a 5′ XbaI site that creates an in-frame reading frame with the upstream CRISPR/CAS9 coding sequence of Example 5, and a downstream XmaI site (SEQ ID NO:21). This XbaI/catalytic-soy-DRM2/XmaI is digested with XbaI and XmaI, purified, and ligated into similarly digested and purified plasmid Insert6 and the remaining steps of Example 5 are followed (The final plasmid used to transform soybean plants is plasmid Insert11 (FIG. 19)).

### Example 9

### Targeting Two LHY-like Promoters in Soybeans with a CRSIPR/CAS9-soybean Truncated DRM2 Fusion Protein in Soybeans

The SbfI to KpnI fragment containing sgRNA1 and sgRNA2 genes is removed from plasmid Insert11 (FIG. 19) and replaced with the SbfI and KpnI digested DNA fragment containing two sgRNA gene cassettes (sgRNA1_LHY) and sgRNA2_LHY) targeted to the two soybean LHY-like promoters (this DNA fragment is described in Example 6; SEQ ID NO:20). The final plasmid used to transform soybean plants is plasmid Insert12 (FIG. 20) and the subsequent steps of Example 5 are followed.

### Example 10

### Crossing of the CCA1-like and LHY-like Methylation Targeted Soybean Plants Comprising a Truncated Soybean DRM2 Fusion Protein

The soybean plants of Example 8 are methylation-targeted for the two CCA1-like promoters and the soybean plants of Example 9 are methylation-targeted for the two LHY-like promoters. Crossing of the two types of plants, and identifying transgenic progeny by PCR analysis of the transgenes (using the unique targeting sequences in each T-DNA are PCR primer sites) containing both types of T-DNAs allows for concurrently methylation of all four CCA1-like and LHY-like promoters in the soybean genome. Progeny plants are phenotypically analyzed and bred as described in Example 5.

### Example 11

### Targeting Two Cc:At-like Promoters in Soybeans with a CRSI_PR/CAS9-soybean Full Length or Truncated Soybean DNA Methyltransferase Fusion Protein in Soybeans

The DNA methyltransferase portion of each CRSIPR/CAS9-DNA methyltransferase fusion protein is encoded by an XbaI to XmaI DNA fragment in Examples 5 and 6. This XbaI to XmaI DNA methyltransferase region can be substituted with other plant DNA methyltransferases to encode other CRSIPR/CAS9-DNA methyltransferase fusion proteins. This substitution is performed at the step that forms binary plasmid Insert7 in Example 5.

For a full length soybean CMT2 (SEQ ID NO:23), this step produces piasmid Insert13 (FIG. 21).

For a truncated soybean CMT2 (SEQ ID NO:25), this step produces plasmid Insert14 (FIG. 22).

For a full length soybean CMT3 (SEQ ID NO:27), this step produces plasmid Insert15 (FIG. 23).

For a truncated soybean CMT3 (SEQ ID NO:29), this step produces plasmid Insert16 (FIG. 24).

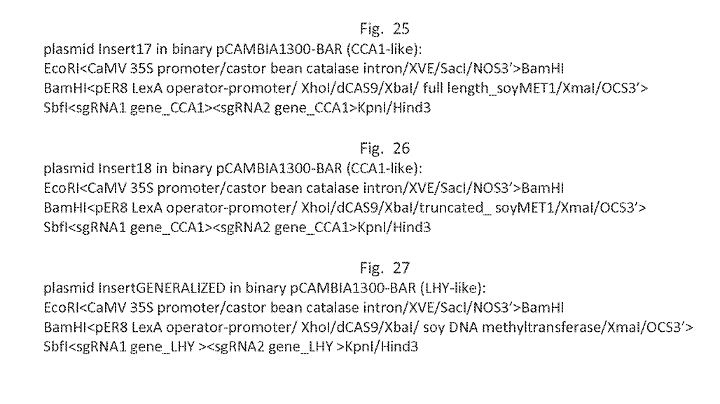

For a full length soybean MET1 (SEQ ID NO:31), this step produces plasmid Insert17 (FIG. 25).

For a truncated soybean MET1 (SEQ ID NO:33), this step produces plasmid Insert18 (FIG. 26).

The subsequent steps are performed as described in Example 5 to produce plants and progeny plants with increased methylation of CCA1-like genes in soybeans.

### Example 12

### Targeting Two LHY-like Promoters in Soybeans with a CRSIPIZ/CAS9-soybean Full Length or Truncated Soybean DNA Methyltransferase Fusion Protein in Soybeans

Each plasmid of plasmid Insert13-18 is digested with SbfI and KpnI, purified, and ligated to SbfI and KpnI digested DNA fragment containing two sgRNA gene cassettes (sgRNA1_LHY) and sgRNA2_LHY) targeted to the two soybean LHY-like promoters (this DNA fragment is described in Example 6; SEQ ID NO:20). The final plasmids have the generalized form of plasmid InsertGENERALIZED (FIG. 27), wherein the soy DNA methyltransferase region comprises a member of the group of full length or truncated CMT2, CMT3, or MET1 soybean DNA methyltransferase coding regions (SEQ ID NO:23-33). The subsequent steps are performed as described in Example 5 to produce plants and progeny plants with increased methylation of LHY-like genes in soybeans.

### Example 13

### Crossing of the CCA1-like and LHY-like Methylation Targeted Soybean Plants Comprising One or More Unique CRISPR/CAS9-DNA Methyltransferase Fusion Proteins

Examples 5-12 produce soybean plants containing a CRISPR/CA S9-DNA methyltransferase fusion protein wherein the DNA methyltransferase domain is a member of the group of DNA methyltransferase proteins consisting of full length or truncated catalytic domains of DRM2, CMT2, CMT3, or MET1. The sgRNA tandem gene cassette region is targeted to either the soybean CCA1-like or the LHY-like promoters. A soybean plant containing a sgRNA tandem cassette targeted to CCA1-like promoters is crossed to a soybean plant containing a sgRNA tandem cassette targeted to LHY-like promoters. The DNA methyltransferase domains in each plant can be the same or different. Crosses wherein the DNA methyltransferases are of different protein families (e.g., DRM2×(CMT2, CMT3, or MET1); CMT2×(CMT3 or MET1); or CMT3×MET1) are useful for recruiting both types of DNA methyltransferase fusion proteins to the same sgRNA target sites, providing both types of DNA methylation activities at both CCA1-like and LHY-like promoters. Crossing of the two types of plants, and identifying transgenic progeny by PCR analysis of the transgenes (using the unique targeting sequences in each T-DNA as PCR primer sites) containing both types of T-DNAs allows for concurrently methylation of all four CCA1-like and LHY-like promoters in the soybean genome with a combination of at least two types of DNA methyltransferase fusion proteins. Alternatively, larger DNA constructs containing both types of DNA methyltransferase fusion proteins or co-transformation with both types can produce plants comprising more than one type of DNA methyltransferase fusion protein. Progeny plants are phenotypically analyzed and bred as described in Example 5.

### Example 14

### Targeting DNA Regions in Different Species and Targeting Different Gene Targets

One skilled in the art will recognize a number of sgRNAs gene cassettes can be made as an array of RNA Pol III promoter cassettes, or a Pol II transcript of one or more sgRNAs, containing targeting homology to one or more regions of the genome of any plant species. The promoters of the CCA1-like and/or MY-like genes encoding these coding regions (identified by BLAST of the protein or nucleotide sequences encoding CCA1-like or LHY-like proteins (including but not limited to Glyma16g01980, Glyma19g45030, Glyma03g42260, Glyma07g05410, Arabidopsis CCA1 NP_850460, Arabidopsis LHY Q6R0H1, XP_002880268, AEB33729, CAD12767, XP_p03528756, XP_008343467, ABW87009, AFO69281). Thus, it is possible to target one or more DNA methyltransferase fusion proteins to most if not all regions of a plant genome that fit the sgRNA targeting criteria.
a. In addition to target sequences in DNA regions to be methylated, it is advantageous to concurrently target promoter regions of genes that produce non-lethal visual phenotypes. Such visual phenotypes provide an indication of the effectiveness of DNA methylation in individual transgenic plants or ancestor plants, allowing for a more effective screening for plants with more efficient DNA methylation, presumably due to more activity of the DNA methyltransferase proteins. In addition to transgenic reporter gene targets such as GFP, GUS, NPTII, or BAR as visual or screenable markers, endogenous genes providing visual phenotypes can be used. Virtually any gene that produces a visual or screenable phenotype (Robertson 2004) can be used as a DNA methylation efficiency indicator, including but not limited to, phytoene desaturase, anthocycanin biosynthetic and regulatory genes, CAB photosynthetic genes, trichome regulatory genes, Chlorophyll biosynthetic genes, cellulose synthase subunit A genes, MSH1, NFL genes, small subunit of ribulose-bisphosphate carboxylaseloxygenase, CTR1 and CTR2, CDPK2, EDS, PS oxygen evolving complex, chalcone synthase, plastid transketolase, acetolactate synthase, protoporphyrin oxidase, glutamine synthetase, RNA polymerase II, catalase 1, magnesium chelatase subunit HAct, NPK1, poly(ADP-ribose) polymerase, SKP1, SGT1, Rar1, Npr1, Ftsh, alpha subunit of 26S proteosome second component of 26S proteosome, CDPK1, RPN3, wound-induced protein kinase, salicylic acid-induced protein kinase, P58 (see (Robertson 2004) fur gene descriptions).

### Example 15

### Targeting CCA1-like and/or LHY-like Promoters by Other DNA Directed DNA Methylase activities

Johnson et. al., (Johnson, Du et al. 2014) describe a method of fusing a DNA binding protein to SUVH2 or SUVH9 containing protein to recruit Pol V and DNA methylases. A DNA binding protein capable of binding to the CCA1-like or LHY-like promoters is fused to the SUVH2 or SUVH9 proteins to direct DNA methylation to these promoters. Plant transformation, screening, and breeding are conducted as described in example 5.

### Example 16

### Changing the Order of the Protein Domains in the Fusion Protein and having Two DNA Methyltransferase Domains in a Single Protein

Those skilled in the art will recognize that the arrangement of the CRISPR/CAS9 and DNA methyltransferase proteins or domains in a fusion protein can be either CRISPR/CAS9-DNA methyltransferase or DNA methyltransferase-CRISPR/CAS9, When two types of DNA methyltransferase activities are expressed within a plant cell, a fusion protein comprising a CRISPR/CAS9, DNA methyltransferase 1, and DNA tneth.yltransferase 2, where the methyltransferases are selected from the group of DRM2, CMT2, CMT3, or MET1 protein families, and the two selected methyltransferases are from different families, is constructed with any order of the CRISPR/CAS9, DNA methyltransferase 1, and DNA methyltransferase 2 positions within the fusion protein. Such fusion proteins can optionally contain an N-terminal or C-terminal NLS for more efficient nuclear localization.

### Example 18

### DNA Methyltransferases from Other Plant and Non-plant Species

Cytosine DNA methyltransferases, preferably those with limited specificity that recognize the CG, CHG, and CHH nt patterns from plant and non-plant species are suitable for the present invention and are identifiable by name or by BLAST homology searches of databases. A native or synthetic DNA sequence is suitable for fusion as a N-terminal or C-terminal fusion with a CRISPR/CAS9 (dCAS) domain for targeting DNA methylation in the presence of a sgRNA guide. Said DNA sequence is inserted into a suitable plant expression vector and transformed into plants, and then the transgenic plants are analyzed and bred as described in Example 5.

### Example 19

### Targeted DNA Methylation in Other Plant Species

The DNA constructs of the above examples are suitable for most plants species. For monocot species, the inclusion of an intron known to increase expression in monocots, such as the rice actin intron, between the promoter and the coding sequence, is advantageous for higher expression levels. Suitable binary vectors are transformed into desired plant species such as corn (Zea mays) by transformation methods known to those skilled in the art. The transformed plants are screened, analyzed, and bred using the procedures described in Example 5.

### REFERENCES

Bae, S. J. Park, et al. (2014). “Cas-OFFinder: a fast and versatile algorithm that searches for potential off-target sites of Cas9 RNA-guided endonucleases.” Bioinformatics 30(10): 1473-1475.
Belhaj, K. A. Chaparro-Garcia, et al. (2013). “Plant genome editing made easy: targeted mutagenesis in model and crop plants using the CRISPR/Cas system.” Plant Methods 9(1): 39.
Cai, M. and Y. Yang (2014). “Targeted genome editing tools for disease modeling and gene therapy.” Curr Gene Ther 14(1): 2-9.
Carroll, D. (2014). “Genome engineering with targetable nucleases.” Annu Rev Biochem 83: 409-439.
Chen, K. and C. Gao (2014). “Targeted genome modification technologies and their applications in crop improvements.” Plant Cell Rep 33(4): 575-583.
Dyachenko, O. V., S. V. Tarlachkov, et al. (2014). “Expression of exogenous DNA methyltransferases: application in molecular and cell biology.” Biochemistry (Mosc) 79(2): 77-87.
Esvelt, K. M., P. Mali, et al. (2013). “Orthogonal Cas9 proteins for RNA-guided gene regulation and editing.” Nat Methods 10(11): 1116-1121.
Fauser, F., S. Schiml, et al. (2014). “Both CRISPR/Cas-based nucleases and nickases can be used efficiently for genome engineering in Arabidopsis thaliana.” Plant J.
Feng, Z., Y. Mao, et al. (2014). “Multigeneration analysis reveals the inheritance, specificity, and patterns of CRISPR/Cas-induced gene modifications in Arabidopsis.” Proc Natl Acad Sci USA 111(12): 4632-4637.
Fichtner, F., R. Urrea Castellanos, et al. (2014). “Precision genetic modifications: a new era in molecular biology and crop improvement.” Planta 239(4): 921-939.
Fonfara, I., A. Le Rhun, et al. (2014). “Phylogeny of Cas9 determines functional exchangeability of dual-RNA and Cas9 among orthologous type II CRISPR-Cas systems.” Nucleic Acids Res 42(4): 2577-2590.
Fu, Y., J. A. Foden, et al. (2013). “High-frequency off-target mutagenesis induced by CRISPR-Cas nucleases in human cells.” Nat Biotechnol 31(9): 822-826.
Fu, Y., J. D. Sander, et al. (2014). “Improving CRISPR-Cas nuclease specificity using truncated guide RNAs.” Nat Biotechnol 32(3): 279-284.
Gao, Y. and Y. Zhao (2014). “Self-processing of ribozyme-flanked RNAs into guide RNAs in vitro and in vivo for CRISPR-mediated genome editing.” J Intear Plant Biol 56(4): 343-349.
Gao, Y. and Y. Zhao (2014). “Specific and heritable gene editing in Arabidopsis.” Proc Natl Acad Sci USA 111(12): 4357-4358.
Gersbach, C. A. and P. Perez-Pinera (2014). “Activating human genes with zinc finger proteins, transcription activator-like effectors and CRISPR/Cas9 for gene therapy and regenerative medicine.” Expert Opin Ther Targets: 1-5.
Hou, Z., Y. Zhang, et al. (2013). “Efficient genome engineering in human pluripotent stem cells using Cas9 from Neisseria meningitidis.” Proc Natl Acad Sci USA 110(39): 15644-15649.
Hsu, P. D., E. S. Lander, et al. (2014). “Development and Applications of CRISPR-Cas9 for Genome Engineering.” Cell 157(6): 1262-1278.
Jackson, R. N., M. Lavin, et al. (2014). “Fitting CRISPR-associated Cas3 into the Helicase Family Tree.” Curr Opin Struct Biol 24: 106-114.
Jao, L. E., S. R. Wente, et al. (2013). “Efficient multiplex biallelic zebrafish genome editing using a CRISPR nuclease system.” Proc Natl Acad Sci USA 110(34): 13904-13909.
Jiang, W., B. Yang, et al. (2014). “Efficient CRISPR/Cas9-Mediated Gene Editing in Arabidopsis thaliana and Inheritance of Modified Genes in the T2 and T3 Generations.” PLoS One 9(6): e99225.
Jiang, W., H. Zhou, et al. (2013). “Demonstration of CRISPR/Cas9/sgRNA-mediated targeted gene modification in Arabidopsis tobacco, sorghum and rice.” Nucleic Acids Res 41(20): e188.
Jinek, M., K. Chylinski, et al. (2012). “A programmable dual-RNA-guided DNA endonuclease in adaptive bacterial immunity.” Science 337(6096): 816-821.
Johnson, L. M., J. Du, et al. (2014). “SRA- and SET-domain-containing proteins link RNA polymerase V occupancy to DNA methylation.” Nature 507(7490): 124-128.
Kim H. and J. S. Kim (2014). “A guide to genome engineering with programmable nucleases.” Nat Rev Genet 15(5): 321-334.
Kunne T., D. C. Swarts, et al. (2014). “Planting the seed: target recognition of short guide RNAs.” Trends Microbiol 22(2): 74-83.
Larson, M. H., L. A. Gilbert, et al. (2013). “CRISPR interference (CRISPRi) for sequence-specific control of gene expression.” Nat Protoc 8(11): 2180-2196.
Li, F., M. Pap,vorth, et al. (2007). “Chimeric DNA methyltransferases target DNA methylation to specific DNA sequences and repress expression of target genes.” Nucleic Acids Res 35(1): 100-112.
Liang, Z., K. Zhang, et al. (2014). “Targeted mutagenesis in Zea mays using TALENs and the CRISPR/Cas system.” J Genet Genomics 41(2): 63-68.
Liu, L. and X. D. Fan (2014). “CRISPR-Cas system: a powerful tool for genome engineering.” Plant Mol Biol 85(3): 209-218.
Lozano-Juste, J. and S. R. Cutler (2014). “Plant genome engineering in full bloom.” Trends Plant Sci 19(5): 284-287.
Ma, S., J. Chang, et al. (2014). “CRISPR/Cas9 mediated multiplex genome editing and heritable mutagenesis of BmKu70 in Bombyx mori.” Sci Rep 4: 4489.
Ma, Y., B. Shen, et al. (2014). “Heritable multiplex genetic engineering in rats using CRISPR/Cas9.” PLoS One 9(3): e89413.
Maeder, M. L., J. F. Angstman, et al. (2013). “Targeted DNA demethylation and activation of endogenous genes using programmable TALE-TET1 fusion proteins.” Nat Biotechnol 31(12): 1137-1142.
Mao, Y., H. Zhang, et al. (2013). “Application of the CRISPR-Cas system for efficient genome engineering in plants.” Mol Plant 6(6): 2008-2011.
McElroy, D., W. Zhang, et al. (1990). “Isolation of an efficient actin promoter for use in rice transformation.” Plant Cell 2(2): 163-171.
Miao, J., D. Guo, et al. (2013). “Targeted mutagenesis in rice using CRISPR-Cas system.” Cell Res 23(10): 1233-1236.
Ng, D. W., M. Miller, et al. (2014), “A Role for CHH Methylation in the Parent-of-Origin Effect on Altered Circadian Rhythms and Biomass Heterosis in Arabidopsis Intraspecific Hybrids.” Plant Cell.
Ni, Z., E. D. Kim, et al. (2009). “Altered circadian rhythms regulate growth vigour in hybrids and allopolyploids.” Nature 457(7227): 327-331.
Nunna, S., R. Reinhardt, et al. (2014). “Targeted methylation of the epithelial cell adhesion molecule (EpCAM) promoter to silence its expression in ovarian cancer cells.” PLoS One 9(1): e87703.
Perez-Pinera, P., D. D. Kocak, et al. (2013). “RNA-guided gene activation by CRISPR-Cas9-based transcription factors.” Nat Methods 10(10): 973-976.
Puchta, H. and F. Fauser (2014). “Synthetic nucleases for genome engineering in plants: prospects for a bright future.” Plant J 78(5): 727-741.
Qi, L. S., M. H. Larson, et al. (2013). “Repurposing CRISPR as an RNA-guided platform for sequence-specific control of gene expression.” Cell 152(5): 1173-1183.
Robertson, D. (2004). “VIGS vectors for gene silencing: many targets, many tools.” Annu Rev Plant Biol 55: 495-519.
Sakurna, T., A. Nishikawa, et al. (2014). “Multiplex genome engir eering in human cells using all-in-one CRISPR/Cas9 vector system.” Sci Rep 4: 5400.
Sander, J. D. and J. K. Joung (2014). “CRISPR-Cas systems for editing, regulating and targeting genomes.” Nat Biotechnol 32(4): 347-355.
Schnable, P. S. and N. M. Springer (2013). “Progress toward understanding heterosis in crop plants.” Annu Rev Plant Biol 64: 71-88.
Shan, Q., Y. Wang, et al. (2013). “Targeted genome modification of crop plants using a CRISPR-Cas system.” Nat Biotechnol 31(8): 686-688.
Siddique, A. N., S. Nunna, et al. (2013). “Targeted methylation and gene silencing of VEGF-A in human cells by using a designed Dnmt3a-Dnmt3L single-chain fusion protein with increased DNA methylation activity.” J Mol Biol 425(3): 479-491.
Sternberg, S. H., S. Redding, et al. (2014). “DNA interrogation by the CRISPR RNA-guided endonuclease Cas9.” Nature 507(7490): 62-67.
Weber, E., R. Gruetzner, et al. (2011). “Assembly of designer TAL effectors by Golden Gate cloning.” PLoS One 6(5): e19722.
Xiao, A., Z. Cheng, et al. (2014). “CasOT: a genome-wide Cas9/gRNA off-target searching tool.” Bioinformatics.
Xie, K., J. Zhang, et al. (2014). “Genome-wide prediction of highly specific guide RNA spacers for CRISPR-Cas9-mediated genome editing in model plants and major crops,” Mol Plant 7(5): 923-926.
Xu, K., C. Ren, et al. (2014). “Efficient genome engineering in eukaryotes using Cas9 from Streptococcus thermophilus.” Cell Mol Life Sci.
Xu, R., H. Li, et al. (2014). “Gene targeting using the Agrobacterium tumefaciens-mediated CRISPR-Cas system in rice.” Rice (NY) 7(1): 5.
Yang, J., M. I. Ordiz, et al. (2012). “A safe and effective plant gene switch system for tissue-specific induction of gene expression in Arabidopsis thaliana and Brassica juncea.” Transgenic Res 21(4): 879-883.
Zhang, H., j. Zhang, et al. (2014). “The CRISPR/Cas9 system produces specific and homozygous targeted gene editing in rice in one generation.” Plant Biotechnol J.
Zuo, J., Q. W. Niu, et al. (2000). “Technical advance: An estrogen receptor-based transactivator XVE mediates highly inducible gene expression in transgenic plants.” Plant J 24(2): 265-273.

What is claimed is:

1. A method of increasing cytosine methylation at one or more targeted DNA sequences in a plant or plant cell comprising the steps of:
a. expressing in a plant or plant cell a DNA methyltransferase fusion protein comptising a DNA methyltransferase domain and a DNA binding domain that binds one or more targeted DNA sequences in said plant or plant cell; and,
b. identifying one or more plants or plant cells, or progeny thereof, with increased DNA methylation at one or more targeted DNA sequences relative to DNA methylation levels of a control plant or plant cell.

2. The method of claim 1, wherein the DNA methyltransferase domain comprises the DNA methyltransferase catalytic domain of a member of the group consisting of CG, CHG, and/or CHH DNA methyltransferase proteins.

3. The method of claim 2, wherein the DNA methyltransferase catalytic domain is selected from the group consisting of members of the MET1, DNMT3a, DNMT3b, DNMT1, DRM2, CMT2, or CMT1/CMT3 families of proteins.

4. The method of claim 1, wherein the DNA methyltransferase catalytic domain is 95% to 100% homologous when aligned to the catalytic domain of a naturally occurring plant DRM2, CMT2, CMT, or MET1 protein, wherein an aligned amino acid position is considered homologous if it contains an amino acid that is identical or a functionally conserved substitution or a conservatively modified variant of the amino acid being compared by alignment.

5. The method of claim 1, wherein the DNA binding domain comprises the DNA binding domain of a member of the group consisting of zinc finger, TALEN, or CRISPR/CAS9, or CRISPR proteins.

6. The method of claim 1, wherein said targeted DNA sequence(s) comprise(s) one or more regions of a CCA1 and/or LHY gene(s).

7. The method of claim 6, wherein CCA1 or LHY genes display increased DNA methylation at one or more promoter or gene regions compared to a control CCA1 or LHY gene.

8. The method of claim I, wherein expressing a DNA methyltransferase fusion protein is accomplished with a transgene comprising an inducible promoter that is operably linked to a DNA methyltransferase fusion protein coding region.

9. The method of claim 1, wherein expressing a DNA methyltransferase fusion protein is accomplished with a transgene comprising a promoter that is operably linked to a DNA methyltransferase fusion protein coding region, wherein said promoter is a member of the group of promoters consisting of MSH1, MET 1, DRM2, CMT1. CMT2, or CMT3 plant promoters.

10. Progeny of a plant or plant cell produced by the method of claim 1.

11. A plant or plant cell comprising one or more DNA methyltransferase fusion proteins comprising a DNA methyltransferase domain and a DNA binding domain that binds one or more targeted DNA sequences in said plant or plant cell.

12. The plant or plant cell of claim 11, wherein the DNA binding domain comprises a CRISPR or CRISPR/CAS9 protein.

13. A plant or plant cell of claim 11, wherein the DNA methyltransferase fusion protein comprises a catalytic methyltransferase domain of a member of the group consisting of a member of the DRM2, CMT2, CMT3. or MET1 family of proteins.

14. The plant or plant cell of claim 13, wherein the DNA methyltransferase fusion protein comprises a DNA binding domain comprising a CRISPR or CRISPR/CAS9 protein.

15. A plant or plant cell of claim 11 comprising at least two types of DNA methyltransferase fusion proteins, wherein each type of DNA methyltransferase fusion protein comprises a DNA methyltransferase domain selected from the DRM2, CMT1, CMT2, CMT3, or MET1 types of DNA methyltransferases.

16. Progeny of the plant or plant cell of claim 11,

17. A plant or plant cell of claim 11 comprising a DNA binding domain that recruits a DNA methylation activity to one or more regions of CCA1 and/or LHY.

18. A DNA construct comprising a DNA methyltransferase fusion protein comprising a DNA methyltransferase domain and a DNA binding domain that binds one or more targeted DNA sequences in a plant or plant cell.

19. A DNA construct of claim 18, wherein the DNA methyltransferase fusion protein comprises a catalytic methyltransferase domain of a member of the group consisting members of the DRM2, CMT2, CMT3, or MET1 family of proteins.

20. A DNA construct of claim 18, wherein the DNA methyltransferase fusion protein comprises a DNA binding domain comprising a CRISPR or CRISPR/CAS9 protein.
